(19) United States
(12) Patent Application Publication
(10) Pub. No.: US 2024/0066075 A1
(43) Pub. Date: Feb. 29, 2024

(54) METHODS AND USES OF MICROBIOME COMPOSITIONS, COMPONENTS, OR METABOLITES FOR TREATING EYE DISORDERS

(71) Applicant: MarvelBiome, Inc., Woburn, MA (US)

(72) Inventors: Jothi Amaranath Govindan, Malden, MA (US); Elamparithi Jayamani, Melrose, MA (US); Priti H. Chatter, Concord, MA (US); Mukesh Chatter, Concord, MA (US)

(21) Appl. No.: 18/244,685

(22) Filed: Sep. 11, 2023

Related U.S. Application Data
(63) Continuation of application No. PCT/US22/20075, filed on Mar. 11, 2022.
(60) Provisional application No. 63/160,452, filed on Mar. 12, 2021.

(51) Int. Cl. A61K 35/742; A61K 31/191; A61K 35/745; A61K 35/747; A61P 27/02; G01N 33/50

(52) U.S. Cl. CPC A61K 35/742; A61K 31/191; A61K 35/745; A61K 35/747; A61P 27/02; G01N 33/5008; C12R 2001/125

(57) ABSTRACT

Methods and uses of compositions (e.g. comprising one or more microbial strains, comprising one or more metabolites (e.g. derived from microbial strains or sources other than microbial strains (e.g. synthetically derived)), comprising one or more components, or a combination thereof) for treating eye disorders are disclosed.

### CROSS REFERENCE TO RELATED APPLICATIONS

The present application claims priority to U.S. Provisional Patent Application No. 63/160,452, filed Mar. 12, 2021, the entire contents of which are hereby incorporated by reference in their entirety.

### BACKGROUND

Many eye diseases, disorders, or conditions including, but not limited to, Age-related macular degeneration (AMD), can cause of blindness. Currently, there are no effective treatments for such diseases, including for AMD, and finding new drugs or treatment methods is a priority.

### SUMMARY

The present disclosure provides an insight that compositions (e.g. microbiome compositions) as described herein may be used to treat diseases, disorders, or conditions (e.g. of the eye (e.g. AMD)) in a subject (e.g. a mammal (e.g. human, mice, etc.)). Among other things, the present disclosure describes technologies that can be used to treat, prevent, and/or reduce the risk of a disease, disorder, or condition (e.g. of the eye). In some embodiments, the present disclosure describes compositions and methods to evaluate the effects of administering such compositions (e.g. microbiome compositions as described herein) to a subject (e.g. an eye of a subject) and/or to identify or characterize effects and/or modulation of levels of metabolites or a metabolome in an eye of a subject upon administration of such compositions. In some embodiments, the metabolites that may be modulated may be associated with certain diseases, disorders, or conditions. In some embodiments, such technologies can be useful to discern metabolite-level differences in a particular subject (e.g., patient) or population (e.g. before and after administration of disclosed compositions). Accordingly, the present disclosure also provides technologies that can be useful to identify and/or assess the nature and effect of disclosed compositions in specific subjects (e.g., patients) and/or populations and thus provide subject-specific information on how to treat a disease, disorder, or condition (e.g. of the eye) in an individual subject or individual population. For example, in some embodiments, technologies provided herein can be useful to identify subject-specific compositions, based on the metabolome in subject-specific samples, and treat and/or prevent a disease, disorder, or condition (e.g. of the eye) by administering disclosed compositions (e.g. subject-specific compositions) (e.g. to modulate subject's metabolome). Thus, technologies described herein may be useful as therapeutics and tools for reducing the risk of certain diseases, disorders, or conditions (e.g. of the eye), and for treating and/or preventing such diseases, disorders, or conditions.

Among other things, the present disclosure provides a method of treating or preventing an eye disorder. In some embodiments, a method comprising administering to a subject a composition comprising one or more microbial strains, components thereof, or metabolites thereof. In some embodiments, a method comprising administering to a subject a composition comprising one or more metabolites. In some embodiments, an eye disorder is Age-related Macular Degeneration (AMD), Geographic atrophy, intermediate AMD, diabetic retinopathy, retinopathy of prematurity, retnitis pigmentosa, retinitis, glaucoma, proliferative vitreoretinopathy, uveitis, keratitis, or scleritis. In some embodiments, an eye disorder is AMD.

In some embodiments, a subject is animal. In some embodiments, a subject is a mammal, e.g., a mammal that experiences or is susceptible to a disease, disorder, or condition as described herein. In some embodiments, an animal is a vertebrate, e.g., a mammal, such as a non-human primate, (particularly a higher primate), a sheep, a dog, a rodent (e.g. a mouse or rat), a guinea pig, a goat, a pig, a cat, a rabbit, or a cow. In some embodiments, an animal is a non-mammal animal, such as a chicken, an amphibian, a reptile, or an invertebrate. In some embodiments, a subject is a human.

In some embodiments, a subject is suffering from or susceptible to one or more eye disorders as described herein. In some embodiments, a subject displays one or more symptoms of one or more eye disorders. In some embodiments, a subject has been diagnosed with one or more eye disorders as described herein. In some embodiments, the subject is receiving or has received certain therapy to diagnose and/or to treat one or more eye disorders.

In some embodiments, one or more microbial strains are from an aminal microbiome. In some embodiments, one or more microbial strains are from a mammalian microbiome. In some embodiments, one or more microbial strains are from a human microbiome. In some embodiments, a human microbiome is a microbiome of a subject.

In some embodiments, one or more components or metabolites (e.g. of one or more microbial strains) are selected from Appendix 1. In some embodiments, metabolites can be from one or more microbial strains. In some embodiments, metabolites can be from a source that is not a microbial strain, e.g., synthetically generated. In some embodiments, one or more components or metabolites (e.g. of one or more microbial strains) is 2-keto-gluconate. In some embodiments, one or more components or metabolites (e.g. of one or more microbial strains) is 5-keto-gluconate. In some embodiments, one or more components or metabolites is Butyrylcamitine, Theobromine, p-Hydroxyphenylpyruvic acid, Propionic acid, Picolinic acid, 2-Hydroxy-4methylvaleric acid, N6-Acetylysine, Urocanic acid, N5-Ethylglutamine, Trigonelline, Stachydrine, Ectoine, 5-Hydroxylysine, Arginine (arg), Cholic acid, 2-(4-Hydroxyphenyl)propionic acid, N-Acetyltryptophan, Hydroxyproline, Argininosuccinic acid, Glutamic acid (Gu), Sarcosine, 5-Methoxyindoleacetic acid, Indole-3-lactic acid, Isovalerylalanine, N-Acetylleucine, I-Methylhistidine, N-Acetylephenylalanine, Proline (Pro), or any combination thereof. In some embodiments, one or more components or metabolites is 4-Hydroxyphenylpyruvic, Ectoine, Gramine, N-Acetyl-L-phenylalanine, Nepsilon-Acetyl-L-lysine, Stachydrine, Trigonelline, 3-Ureidopropionic acid, Theobromine, Hippuric acid, Imidazolepropionic acid, NG-Methyl-L-arginine, trans-Urocanic Acid, N-Acetyl-L-leucine, Sarcosine, Isobutyrylcarnitine, b-Hydroxyisovaleric acid, L-Theanine/N5-Ethylglutamine, 5-Hydroxylysine, Phenaceturic acid, betaine, hydroxyproline, Picolinic acid, 2-Aminoadipic acid, Glycerophosphocholine, camitine, Glycerol 3-phosphate, Argininosuccinic acid, creatine, Terephthalic acid, Homocitrulline, Mucic acid, Homocysteinesulfinic acid, Trimethyllysine, Spermidine, Glyoxylic acid, XA0013 C6H6O4S, 3-Indoxylsulfuric acid, Nicotinamide, N-Formyiglycine, Ureidoglycolate, N-Methylproline, Glucaric acid, Butyrylcamitine, Methionine sulfoxide, Carboxymethyllysine, Glycolic acid, Phenaceturic acid, Diethanolamine, Phosphorylcholine, Guanidinosuccinic acid, N-Acetylhistidine, Glyceric acid, S-Methylmethionine. Cysteine glutathione disulfide, Kynurenine, N-Acetylphenylalanine, Threonic acid. Malic acid, 7,8-Dihydrobiopterin, Homovanillic acid, Taurocholic acid, 5-Methoxyindoleacetic acid, butyrate, b-Hydroxyisovaleric acid, 2-Oxoglutaric acid, N-Acetyltryptophan, Thiaproline, Hypotaurine, Cholic acid, Acetoacetic acid, Ethanolamine, Guanidoacetic acid, S-Sulfocysteine, Myristic acid C14:0 XA0027, or my combination thereof.

In some embodiments, one or more microbial strains are Gluconacetobacter hansenii, Terrisporobacter glycolicus, Coprococcus sp., Lactobacillus plantarum, Clostridium hutyricum, Paenihacillussp., Veillonella. sp., Rbfidohacterium. sp., Racillus subtilis, Acidaminococcus sp., or a combination thereof. In some embodiments, one or more microbial strains are Gluconacelobacler hanseni, Terrisporobacter glycolicus, Coprococcus sp., Lactobacillus plantarum, Veillonella atypica, Bifdobacterium, or a combination thereof. In some embodiments, a microbial strain is Bacillus subtilis.

In some embodiments, a composition comprises two or more microbial strains. In some embodiments, a composition comprises five or more microbial strains. In some embodiments, a composition comprises ten or more microbial strains.

In some embodiments, a composition is administered topically, orally, opthalmically, intravitreally, or suprachoroidally. In some embodiments, a composition is administered orally. In some embodiments, a composition is administered opthalmically.

In some embodiments, a composition is formulated as a syrup, a liquid, a tablet, a troche, a gummy, a capsule, a powder, a gel, a film, an injection, or an eye drop.

In some embodiments, each microbial strain of one or more microbial strains in a composition is available at a concentration from 10¹ to 10¹⁵ CFU. In some embodiments, each microbial strain of one or more microbial strains in a composition is available at a concentration of at least 10⁶ CFU. In some embodiments, each microbial strain of one or more microbial strains in a composition comprises 10¹ colony forming units (CFUs) to 10²⁰ CFU. In some embodiments, each microbial strain of one or more microbial strains in a composition comprises 10¹ colony forming units (CFUs) to 10¹ CFU. In some embodiments, each microbial strain of one or more microbial strains in a composition comprises 10⁶ CFU to 10¹⁵ CFUs. In some embodiments, each microbial strain of one or more microbial strains in a composition comprises about 10¹ CFU to 10¹⁵ CFU, or about 10² CFU to 10¹⁴ CFU, or about 10³ CFU to 10¹³ CFU, or about 10⁴ CFU to 10¹³ CFU, or about 10⁵ CFU to 10¹² CFU, or about 10⁶ CFU to 10¹¹ CFU, or about 10⁷ CFU to 10¹⁰ CFU, or about 10⁸ CFU to 10⁹ CFU, or about 10⁵ CFU to 10¹⁰ CFU, or about 10⁸ CFU to 10¹² CFU. In some embodiments, each microbial strain of one or more microbial strains in a composition comprises at least about 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², or more CUs. In some embodiments, each of one or more microbial strains in a composition comprises at most about 10¹⁵, 5×10¹⁴, 10¹⁴, 5×10¹³, 10¹³, 5×10¹², 10¹², 5×10¹¹, 10¹¹, 5×10¹⁰, 10¹⁰, 5×10⁹, 10⁹, 5×10⁸, 10⁸, or less CFUs. In some embodiments, each microbial strain of one or more microbial strains in a composition comprises same number of CFUs. In some embodiments, some microbial strains of one or more microbial strains in a composition comprises a different number of CFUs.

The present disclosure provides, among other things, a composition comprising one or more microbial strains, components thereof, or metabolites thereof, wherein a composition is for treating an eye disorder. In some embodiments, a composition, as described herein, comprises one or more metabolites (e.g. derived from sources other than microbial strains (e.g. synthetically derived)), wherein the composition is for treating an eye disorder.

The present disclosure provides a composition comprising one or more microbial strains selected from Gluconacetobacter hansenii, Terrisporobacter glycolicus, Coprocccus sp., Lactobacillus plantarum, Clostridium butyrican, Paenibacillus sp., Veillonella sp., Bifdobacterium sp., Bacillus subtilis, Acidaminococcus sp., or a combination thereof. In some embodiments, a composition comprises one or more microbial strains selected from Gluconacetobacter hanseni, Terrisporobacter glvcohcus, Coprococcus sp., Lactobacillus plantarum, Veillonella atypica, Bifdobacterium, or a combination thereof. In some embodiments, a composition comprises a microbial strain. In some embodiments, a microbial strain is Bacillus subtilis. In some embodiments, a composition comprises at least two microbial strains selected from a group consisting of Gluconacetobacter hansenti, Terrisporobacter glycolicus, Coprococcus sp., Lactobacillus plantarum, Clostridium butyricum, Paenibacillus sp., Veillonella sp., Bifidobacterium sp., Bacillus subtilis, Acidaminococcus sp., or a combination thereof. In some embodiments, a composition comprises at least two microbial strains selected from a group consisting of Gluconacetobacter hanseni, Terrisporobacter glycolicus, Coprococcus sp., Lactobacillus plantarum, Veillonella atypica, Bfidobacterium, or a combination thereof. In some embodiments, a composition comprises at least five microbial strains selected from a group consisting of Gluconacetobacter hansenii, Terrisporobacter glycolicus, Coprococcus sp., Lactobacillus plantarum, Clostridium butyricum, Paenibacillus sp., Veillonella sp., Bifidobacterium sp., Bacillus subtilis, Acidaminococcus sp., or a combination thereof. In some embodiments, a composition comprises at least five microbial strains selected from a group consisting of Gluconacetobacter hansenm, Terrisporobacter glycolicus, Coprococcus sp., Lactobacillus plantarum, Veillonella atypica, Biftdobacterium, or a combination thereof. In some embodiments, a composition comprises or consists of Gluconacetobacter hansenii. Terrisporobacter glycolicus, Coprococcus sp., Lactobacillus plantarum, Clostridiwn butyricum, Paenibacillus sp., Veillonella sp., Bftdobacterium sp., Bacillus subtilis, Acidaminococcus sp.. In some embodiments, a composition comprises or consists of Gluconacetobacter hanseni, Terrisporobacter glycolicus, Coprococcus sp., Lactobacillus plantarum, Veillonella atypica, Bifdobacterium.

In some embodiments, a composition is for topical, oral, opthalmical, intravitreal, or suprachoroidal administration. In some embodiments, a composition is for oral administration. In some embodiments, a composition is opthalmical administration.

The present disclosure provides that a composition as described herein is for modulating one or more metabolites in a subject.

The present disclosure provides that a composition as described herein is for use in characterizing an ability of one more microbial strains to modulate one or more metabolites in a subject.

The present disclosure provides that a use of a composition as described herein is for treating or ameliorating a disease, disorder, or condition in a subject, wherein a disease, disorder, or condition is associated with one or more metabolites.

In some embodiments, a use of a composition as described herein is for treating or ameliorating an eye disorder. In some embodiments, a use of a composition as described herein is for treating or ameliorating a disease, disorder, or condition selected from AMD, Geographic atrophy, intermediate AMD, diabetic retinopathy, retinopathy of prematurity, retnitis pigmentosa, retinitis, glaucoma, proliferative vitreoretinopathy, uveitis, keratitis, or scleritis. In some embodiments, a use of a composition as described herein is for treating or ameliorating AMD.

The present disclosure provides a method of screening a microbial strain, comprising contacting a microbial strain to a culture comprising RPE cells that model AMD, and determining whether a microbial strain altered a feature of a culture, wherein a feature is associated with AMD.

In some embodiments, a step of determining comprises comparing a feature before and after performance of the step of contacting. In some embodiments, a step of determining comprises comparing a feature after the step of contacting with a comparable reference.

In some embodiments, a comparable reference is a historical reference. In some embodiments, a comparable reference is a negative control reference. In some embodiments, a comparable reference is a positive control reference.

In some embodiments, a feature is a level of cell viability. In some embodiments, a feature is level or activity of a nucleic acid or protein, or form thereof. In some embodiments, a feature is oxidative stress. In some embodiments, a feature is ATP levels. In some embodiments, a feature is inflammation.

The present disclosure provides a method of characterizing a microbial strain, comprising adding a microbial strain to a culture comprising RPE cells that model AMD, and determining whether a microbial strain affects one or more parameters of RPE cells, wherein one or more parameters are associated with AMD.

The present disclosure provides a method of manufacturing a pharmaceutical treatment for an eye comprising characterizing one or more microbial strains, components, or metabolites thereof comprising the steps of adding a microbial strain to a culture comprising RPE cells that model AMD, and determining whether a microbial strain affects one or more parameters of RPE cells, wherein one or more parameters are associated with AMD.

The present disclosure provides a method of assessing a microbial strain for an ability to one or more parameters of a culture, comprising adding a microbial strain to a culture comprising RPE cells that model AMD, and determining whether a microbial strain affects one or more parameters of RPE cells, wherein one or more parameters are associated with AMD.

In some embodiments, a method further comprises before adding a microbial strain to a culture, determining one or more parameter values of RPE cells in a culture; after adding a microbial strain to a culture, determining the same one or more parameter values of RPE cells in a culture; and comparing one or more parameter values determined before adding a microbial strain with one or more parameter values determined after adding a microbial strain.

In some embodiments, a one or more parameters includes: (i) viability of cells; (ii) level or activity of a nucleic acid or protein, or form thereof; (iii) oxidative stress; (iv) ATP levels; (v) inflammation; or (vi) a combination thereof.

The present disclosure provides that a composition as described herein is for use in treating or preventing an eye disorder, comprising one or more microbial strains, components thereof, or metabolites thereof. In some embodiments, a composition, as described herein, is for use in treating or preventing an eye disorder, comprising one or more metabolites (e.g. derived from sources other than microbial strains (e.g. synthetically derived)).

The present disclosure provides that a composition as described herein is for use in treating or preventing an eye disorder, comprising one or more microbial strains, components thereof, or metabolites thereof, wherein a one or more components or metabolites (e.g. of a one or more microbial strains) are selected from Appendix 1. The present disclosure further provides that a composition as described herein is for use in treating or preventing an eye disorder, comprising one or more components or metabolites, which can be selected from Appendix 1.

In some embodiments, metabolites can be from one or more microbial strains. In some embodiments, metabolites can be from a source that is not a microbial strain, e.g., synthetically generated. In some embodiments, a one or more components or metabolites (e.g. of one or more microbial strains) is 2-keto-gluconate. In some embodiments, a one or more components or metabolites (e.g. of one or more microbial strains) is 5-keto-gluconate. In some embodiments, one or more components or metabolites is Butyrylcamitine, Theobromine, p-Hydroxyphenylpyruvic acid, Propionic acid, Picolinic acid, 2-Hydroxy-4methylvaleric acid, N6-Acetylysine, Urocanic acid, N5-Ethylglutamine, Trigonelline, Stachydrine, Ectoine, 5-Hydroxylysine, Arginine (arg), Cholic acid, 2-(4-Hydroxyphenyl)propionic acid, N-Acetyltryptophan, Hydroxyproline, Argininosuccinic acid, Glutamic acid (Glu), Sarcosine, 5-Methoxvindoleacetic acid, Indole-3-lactic acid. Isovalerylalanine, N-Acetylleucine, 1-Methylhistidine, N-Acetylephenylalanine, Proline (Pro), or any combination thereof. In some embodiments, one or more components or metabolites is 4-Hydroxyphenylpyruvic, Ectoine, Gramine, N-Acetyl-L-phenylalanine, Nepsilon-Acetyl-L-lysine, Stachydrine, Trigonelline, 3-Ureidopropionic acid, Theobromine, Hippuric acid, Imidazolepropionic acid, NG-Methyl-L-arginine, trans-Urocanic Acid, N-Acetyl-L-leucine, Sarcosine, Isobutyrylcanitine, b-Hydroxyisovaleric acid, L-Theanine/N5-Ethylglutamine, 5-Hydroxylysine, Phenaceturic acid, betaine, hydroxyproline, Picolinic acid, 2-Aminoadipic acid, Glycerophosphocholine, camitine, Glycerol 3-phosphate, Argininosuccinic acid, creatine, Terephthalic acid, Homocitrulline, Mucic acid, Homocysteinesulfmic acid, Trimethyllysine, Spermidine, Glyoxylic acid. XA0013 C6H6O4S, 3-Indoxylsulfuric acid, Nicotinamide, N-Formylglycine, Ureidoglycolate, N-Methylproline, Glucaric acid, Butvrylcamitine, Methionine sulfoxide, Carboxymethyllysine, Glycolic acid, Phenaceturic acid, Diethanolamine. Phosphorylcholine, Guanidinosuccinic acid, N-Acetylhistidine, Glyceric acid, S-Methylmethionine, Cysteine glutathione disulfide, Kynurenine, N-Acetylphenylalanine, Threonic acid, Malic acid, 7,8-Dihydrobiopterin, Homovanillic acid, Taurocholic acid, 5-Methoxyindoleacetic acid, butyrate, b-Hydroxyisovaleric acid, 2-Oxoglutaric acid, N-Acetyltryptophan. Thiaproline, Hypotauine, Cholic acid, Acetoacetic acid, Ethanolamine, Guanidoacetic acid, S-Sulfocysteine, Myristic acid C14:0 XA0027, or any combination thereof.

In some embodiments, a composition as descnbed herein is for use in treating or preventing an eye disorder, comprising one or more microbial strains, components thereof, or metabolites thereof and comprises one or more microbial strains selected from Gluconacetobacter hansenii, Terrisporobacter glycolicus, Coprococcus sp., Lactobacillus plantarum, Clostridium butyricun, Paenibacillus sp., Veillonella sp., Bifidobacterium sp., Bacillus subtilis, Acidaminococcus sp., or a combination thereof. In some embodiments, a composition as described herein is for use as described herein and comprises one or more microbial strains selected from Gluconacetobacter hanseni, Terrnsporobacter glycolicus, Coprococcus sp., Lactobacillus plantarum, Veillonella atypica, Bifidobacterium, or a combination thereof. In some embodiments, a composition as described herein is for use as described herein and comprises a microbial strain. In some embodiments, a composition as described herein is for use as described herein and comprises a microbial strain is Bacillus subtilis. In some embodiments, a composition as described herein is for use as described herein and comprises at least two microbial strains selected from a group consisting of Gluconacetobacter hansenii, Terrisporobacter glycolicus, Coprococcus sp., Lactobacillus plantarum, Clostridium butyricum, Paenibacillus sp., Veillonella sp., Bifdobacterium sp., Bacillus subtilis, Acidaminococcus sp., or a combination thereof. In some embodiments, a composition as described herein is for use as described herein and comprises at least two microbial strains selected from a group consisting of Gluconacetobacter hanseni, Terrisporobacter glycolicus, Coprococcus sp., Lactobacillus plantarum, Veillonella atypica, Bifidobacternum, or a combination thereof. In some embodiments, a composition as described herein is for use as described herein and comprises at least five microbial strains selected from a group consisting of Gluconacetobacter hansenii, Terrisporobacter glycolicus, Coprococcus sp., Lactobacillus plantarum, Clostridium butyricum, Paenibacillus sp., Veillonella sp., Bifidobacterium sp., Bacillus subtilis, Acidaminococcus sp., or a combination thereof. In some embodiments, a composition as described herein is for use as described herein and comprises at least five microbial strains selected from a group consisting of Gluconacetobacter hanseni, Terrnsporobacter glycolicus, Coprococcus sp., Lactobacillus plantarum, Veillonella atypica, Btidobacterum, or a combination thereof. In some embodiments, a composition as described herein is for use as described herein and comprises or consists of Gluconacetobacter hansenii, Terrisporobacter glycolicus, Coprococcus sp., Lactobacillus plantarum, Clostridium butyricum, Paenibacillus sp., Veillonella sp., Bifidobacterium sp., Bacillus subtilis, Acidaminococcus sp.. In some embodiments, a composition as described herein is for use as described herein and comprises or consists of Gluconaceobacter hanseni, Terrisporobacter glycolicus, Coprococcus sp., Lactobacillus plantarum, Veillonella atypica, Bifidobacterium.

The present disclosure provides an eye drops comprising a composition as described herein.

The present disclosure provides a kit comprising a composition as described herein for use in treating or preventing an eye disorder as described herein.

These, and other aspects encompassed by the present disclosure, are described in more detail below and in the claims.

### Definitions

The scope of the present invention is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Administration: As used herein, the term “administration” typically refers to the administration of a composition to a subject or system to achieve delivery of an agent to the subject or system. In some embodiments, the agent is, or is included in, the composition; in some embodiments, the agent is generated through metabolism of the composition or one or more components thereof. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e g, intrahepatic), mucosal, nasal, oral, rectal, subcutaneous, sublingual, topical, tracheal (e.g., by intratracheal instillation), vaginal, vitreal, etc. In many embodiments provided by the present disclosure, administration is oral administration. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. Administration of cells can be by any appropriate route that results in delivery to a desired location in a subject where at least a portion of the delivered cells or components of the cells remain viable. A period of viability of cells after administration to a subject can be as short as a few hours, e.g., twenty-four hours, to a few days, to as long as several years, i.e., long-term engraftment. In some embodiments, administration comprises delivery of a bacterial extract or preparation comprising one or more bacterial metabolites and/or byproducts but lacking fully viable bacterial cells.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Approximately: As applied to one or more values of interest, includes to a value that is similar to a stated reference value. In certain embodiments, the term “approximately” or “about” refers to a range of values that fall within ±10% (greater than or less than) of the stated reference value unless otherwise stated or otherwise evident from the context (except where such number would exceed 100% of a possible value).

Comparable: As used herein, the term “comparable” refers to two or more agents, entities, situations, sets of conditions, subjects, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Conservative: As used herein, refers to instances when describing a conservative amino acid substitution, including a substitution of an amino acid residue by another amino acid residue having a side chain R group with similar chemical properties (e.g., charge or hydrophobicity). In general, a conservative amino acid substitution will not substantially change the functional properties of interest of a protein, for example, the ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr. F/Y), lysine/arginine (Lys/Arg, K/R), alanine/valine (Ala/Val, AN), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet, G. H. et al., 1992, Science 256:1443-1445, which is incorporated herein by reference in its entirety. In some embodiments, a substitution is a moderately conservative substitution wherein the substitution has a nonnegative value in the PAM250 log-likelihood matrix.

CONSERVATIVE AMINO ACID SUBSTITUTIONS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S—Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid (Kauer, |
| | | U.S. Pat. No. (4,511,390) |
| Serine | S | D-Ser, Thr, D-Thr, allo-Thr, Met, D-Met, Met |
| | | (O), D-Met (O), L-Cys, D-Cys |
| Threonine | T | D-Thr, Ser, D-Ser, allo-Thr, Met, D-Met, Met |
| | | (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

Control: As used herein, refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. A “control” also includes a “control animal.” A “control animal” may have a modification as described herein, a modification that is different as described herein, or no modification (i.e., a wild-type animal). In one experiment, a “test” (i.e., a variable being tested) is applied. In a second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Determining, measuring, evaluating, assessing, assaying and analyzing Determining, measuring, evaluating, assessing, assaying and analyzing are used interchangeably herein to refer to any form of measurement, and include determining if an element is present or not. These terms include both quantitative and/or qualitative determinations. Assaying may be relative or absolute. “Assaying for the presence of” can be determining the amount of something present and/or determining whether or not it is present or absent.

Dosage form: Those skilled in the art will appreciate that the term “dosage form” may be used to refer to a physically discrete unit of an agent (e.g., a therapeutic agent) for administration to a subject. Typically, each such unit contains a predetermined quantity of agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population.

Engineered: In general, the term “engineered” refers to the aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, refers to an inactive (e.g., non-therapeutic) agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Functional: As used herein, a “functional” biological molecule is a biological molecule in a form in which it exhibits a property and/or activity by which it is characterized. A biological molecule may have two functions (i.e., bifunctional) or many functions (i.e., multifunctional).

Gene: As used herein, refers to a DNA sequence in a chromosome that codes for a product (e.g., an RNA product and/or a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). For the purpose of clarity, we note that, as used in the present disclosure, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid.

Improve, increase, enhance, inhibit or reduce: As used herein, the terms “improve,” “increase.” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Isolated: As used herein, refers to a substance and/or entity that has been (1) separated from at least some of the components with which it was associated when initially produced (whether in nature and/or in an experimental setting), and/or (2) designed, produced, prepared, and/or manufactured by the hand of man. In some embodiments, an isolated substance or entity may be enriched; in some embodiments, an isolated substance or entity may be pure. In some embodiments, isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “enriched”. “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. Those skilled in the art are aware of a variety of technologies for isolating (e.g., enriching or purifying) substances or agents (e.g., using one or more of fractionation, extraction, precipitation, or other separation).

Level: As used herein, the term “level” refers to a scale of amount or quantity of a substance (e.g., a metabolite). In some embodiments, a level can be simply the presence or absence of a substance. A level of a substance may be represented in multiple ways or formats. For example, in some embodiments, a level may be represented as a percentage (%), a measure of weight (e.g., mg, μg, ng, etc.), a measure of concentration (e.g., mg/mL, μg/mL, ng/mL, etc.), a measure of volume (e.g., mL, μL, nL, etc.), in % change, etc.

Metabolite: As used herein, the term “metabolite” refers to a substance (e.g., a small molecule, macromolecule, organic compound, or inorganic compound) made or used during metabolism. Metabolism is generally understood as a process by which a substance (e.g., food, drug, chemical, cell, or tissue) is chemically broken down. In some embodiments, a metabolite is an end product. In some embodiments, a metabolite is an intermediate. Exemplary metabolites are provided herein, e.g., in Appendix 1-1. Exemplary metabolic pathways are provided herein, e.g., in Appendix 1-2.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: ophthalmic administration, intravitreal administration, suprachoroidal administration, oral administration, for example, drenches (aqueous or non-aqueous solutions or suspensions), tablets, e.g., those targeted for buccal, sublingual, and systemic absorption, boluses, powders, granules, pastes for application to the tongue, capsules, powders, etc. In some embodiments, an active agent may be or comprise a cell or population of cells (e.g., a culture, for example of an Ellagitannin-Enzyme-Synthesizing (EES) microbe); in some embodiments, an active agent may be or comprise an extract or component of a cell or population (e.g., culture) of cells. In some embodiments, an active agent may be or comprise an isolated, purified, or pure compound. In some embodiments, an active agent may have been synthesized in vitro (e.g., via chemical and/or enzymatic synthesis). In some embodiments, an active agent may be or comprise a natural product (whether isolated from its natural source or synthesized in vitro).

Pharmaceutically acceptable: As used herein, the term “pharmaceutically acceptable” which, for example, may be used in reference to a carrier, diluent, or excipient used to formulate a pharmaceutical composition as disclosed herein, means that the carrier, diluent, or excipient is compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be is “acceptable” in the sense of being compatible with the other ingredients of the formulation and not injurious to the subject (e.g., patient). Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide: alginic acid; pyrogen-free water; isotonic saline; Ringer's solution, ethyl alcohol; pH buffered solutions; polyesters, polycarbonates and/or polyanhydrides; and other non-toxic compatible substances employed in pharmaceutical formulations.

Prebiotic: As used herein, a “prebiotic” refers to an ingredient that allows or promotes specific changes, both in the composition and/or activity in the gastrointestinal microbiota that may (or may not) confer benefits upon the host. In some embodiments, a prebiotic can include one or more of the following: the prebiotic comprises a pome extract, berry extract and walnut extract.

Prevention: The term “prevention”, as used herein, refers to a delay of onset, and/or reduction in frequency and/or severity of one or more symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete, for example, when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90, or up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, magma, sputum, synovial fluid, sweat, tears, urine, vaginal secretions, vitreous humour, vomit, plasma, mucous, digestive fluid, stool, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Small molecule: As used herein, the term “small molecule” refers to small organic or inorganic molecules of molecular weight below about 3,000 Daltons. In general, small molecules may have a molecular weight of less than 3,000 Daltons (Da). Small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1.500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

Subject: As used herein, the term “subject” refers to an individual to which a provided treatment is administered. In some embodiments, a subject is animal. In some embodiments, a subject is a mammal, e.g., a mammal that experiences or is susceptible to a disease, disorder, or condition as described herein. In some embodiments, an animal is a vertebrate, e.g., a mammal, such as a non-human primate, (particularly a higher primate), a sheep, a dog, a rodent (e.g. a mouse or rat), a guinea pig, a goat, a pig, a cat, a rabbit, or a cow. In some embodiments, an animal is a non-mammal animal, such as a chicken, an amphibian, a reptile, or an invertebrate model C. elegans. In some embodiments, a subject is a human. In some embodiments, a subject is suffering from or susceptible to one or more diseases, disorders or conditions as described herein. In some embodiments, a subject displays one or more symptoms of a one or more diseases, disorders or conditions as described herein. In some embodiments, a subject has been diagnosed with one or more diseases, disorders or conditions as described herein. In some embodiments, the subject is receiving or has received certain therapy to diagnose and/or to treat a disease, disorder, or condition. In another embodiment, the subject is an experimental animal or animal substitute as a disease model.

Substantially: As used herein, refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to subjects (e.g., patients) in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### BRIEF DESCRIPTION OF THE DRAWING

FIG. 1 shows absorbance data representative of cell viability of human retinal pigment epithelial cells (ARPE-19) when treated with various doses of NaIO₃ compared to mock treatment. Cell viability was assessed using the 3-(4,5-dimethylthiazol-2-yl)-2,5-diphenyl tetrazolium bromide (MTT) assay. Each dot in the figure indicates technical replicates.

FIG. 2 shows absorbance data representative of cell viability of ARPE-19 cells when treated with various microbiome therapies (MBTs) numbered 1 to 10 compared to mock treatment (positive and negative controls). Cell viability was assessed using the MTT assay. Each dot in the figure indicates technical replicates from two independent trials.

FIG. 3 shows absorbance data representative of cell viability of ARPE-19 cells when treated with MBT CT6 compared to mock treatment (positive and negative controls). CT6 is a combination of Gluconacetobacter hanseni. Terrisporobacter glycolicus. Coprococcus sp., Lactobacillus plantarum, Vellonella atypica, and Bifidobacterium. Cell viability was assessed using the MTT assay. Each dot in the figure indicates technical replicates from two independent trials.

FIG. 4 shows absorbance data representative of cell viability of ARPE-19 cells when treated with a metabolite, 2-keto-gluconate, compared to mock treatment (positive and negative controls). Cell viability was assessed using the MTT assay. Each dot in the figure indicates technical replicates.

FIG. 5 shows absorbance data representative of cell viability of ARPE-19 cells when treated with a metabolite, 5-keto-gluconate, compared to mock treatment (positive and negative controls). Cell viability was assessed using the MTT assay. Each dot in the figure indicates technical replicates.

### DETAILED DESCRIPTION OF CERTAIN EMBODIMENTS

Age-Related Macular Degeneration

The macula is a small area in the retina of the eye, approximately 3 to 5 millimeters in size, adjacent to the optic nerve. It is the most sensitive area of the retina and contains the fovea, a depressed region that allows for high visual acuity and contains a dense concentration of cones, the photoreceptors that are responsible for color vision.

Macular degeneration is a term that refers to a number of different diseases characterized by degenerative changes in the macula, all of which leads to a loss of central vision. Age-related macular degeneration (AMD) is the most common cause of functional blindness in developed countries for those over 50 years of age (Seddon, J M. Epidemiology of age-related macular degeneration. In: Ogden, T E, et al., eds. Ryan SJ, ed-in-chief. Retina Vol II. 3rd ed. St. Louis, MO: Mosby; 2001:1039-50, which is incorporated in its entirety by reference herein). The disease is characterized by progressive degeneration of the retina, retinal pigment epithelium (RPE), and underlying choroid (the highly vascular tissue that lies beneath the RPE, between the retina and the sclera). The retinal pigment epithelial layer is believed to be crucial for photoreceptor health. Cells in this layer recycle visual pigment (rhodopsin), phagocytose photoreceptor tips daily as part of rod and cone regeneration, and transport fluid across the membrane to the choroid, which is believed to help prevent detachment of the neural retina. Central vision deteriorates when cells in the RPE cease to function properly, which can lead to photoreceptor degeneration.

A variety of factors including oxidative stress, inflammation with a possible autoimmune component, genetic background (e.g., mutations), and environmental or behavioral factors such as smoking and diet may contribute to the pathogenesis of AMD in ways that are as yet not fully understood. Regardless of the underlying etiology, a clinical hallmark of AMD is the appearance of drusen, localized deposits of lipoproteinaceous material that accumulate in the space between the RPE and Bruch's membrane, which separates the RPE from the choroidal vessels (choriocapillaris). Drusen are typically the earliest clinical finding in AMD, and the existence, location, and number of drusen are used in classifying the disease into stages and for monitoring its progression (Ambati, J., et al., Surv. Ophthalmol., 48(3): 257-293, 2003; “Preferred Practice Pattern: Age-Related Macular Degeneration”. American Academy of Ophthalmology, 2003, which is incorporated in its entirety by reference herein). Drusen are typically the earliest clinical finding in AMD.

AMD has been classified into both “dry” and “wet” (exudative, or neovascular) forms. Dry AMD is much more common than wet AMD, but the dry form can progress to the wet form, and the two occur simultaneously in a significant number of cases. Dry AMD is typically characterized by progressive apoptosis of cells in the RPE layer, overlying photoreceptor cells, and frequently also the underlying cells in the choroidal capillary layer. Confluent areas (typically at least 175 μm in minimum diameter) of RPE cell death accompanied by overlying photoreceptor atrophy are referred to as geographic atrophy (GA). Patients with this form of AMD experience a slow and progressive deterioration in central vision.

Wet AMD is characterized by bleeding and/or leakage of fluid from abnormal vessels that have grown from the choroidal vessels (choriocapillaris) beneath the RPE and the macula, which can be responsible for sudden and disabling loss of vision. It has been estimated that much of the vision loss that patients experience is due to such choroidal neovascularization (CNV) and its secondary complications. A subtype of neovascular AMD in which angiomatous proliferation originates from the retina and extends posteriorly into the subretinal space, eventually communicating in some cases with choroidal new vessels has been identified (Yannuzzi, L. A., et al., Retina, 21(5):416-34, 2001, which is incorporated in its entirety by reference herein), his form of neovascular AMD, termed retinal angiomatous proliferation (RAP) can be particularly severe. The existence of macular drusen is a strong risk factor for the development of both wet and dry forms of AMD (Ambati, J., et al., supra).

Treatment options for AMD are limited, and none are fully effective (Ambati, J., et al., Surv. Ophthalmol., 48(3): 257-293, 2003, and references therein, which are incorporated in their entirety by reference herein). Although the implementation of anti-VEGF treatment seems to be decreasing the prevalence of AMD, it is predicted that the number of affected persons will still increase in the next two decades (Colijn et al., Ophthalmol., 124 (12), 1753-1763, 2017, which is incorporated in its entirety by reference herein). To further decrease the prevalence of AMD, discovering the treatment options for dry AMD seems to be the appropriate solution since it remains untreatable. Thus, there is a need for new approaches to the treatment of AMD and also of other diseases and conditions of the eye characterized by macular degeneration, choroidal neovascularization, retinal neovascularization, retinal angiomatous proliferation, and/or blood vessel leakage. Such diseases and conditions include, but are not limited to, diabetic retinopathy and retinopathy of prematurity. There is also a need for new approaches to the treatment of eye disorders characterized by ocular inflammation.

The present disclosure provides compositions and methods for treatment of eye disorders characterized by macular degeneration, choroidal neovascularization (CNV), retinal neovascularization (RNV), ocular inflammation, or any combination of the foregoing. The phrase “characterized by” is intended to indicate that macular degeneration, CNV, RNV, and/or ocular inflammation is a characteristic (i.e., typical) feature of the disorder. Macular degeneration, CNV, RNV, and/or ocular inflammation may be a defining and/or diagnostic feature of the disorder. Exemplary disorders that are characterized by one or more of these features and can be treated with the compositions (e.g. microbiome compositions) and methods disclosed herein include, but are not limited to, macular degeneration related conditions, diabetic retinopathy, retinopathy of prematurity, retnitis pigmentosa, retinitis, glaucoma, proliferative vitreoretinopathy, uveitis, keratitis, and scleritis. As mentioned above, macular degeneration refers to a variety of degenerative conditions characterized by central visual loss due to deterioration of the macula. The most common of these conditions is age related macular degeneration (AMD), which exists in both “dry” and “wet” forms.

Ocular inflammation can affect a large number of eye structures including the conjunctiva, cornea, episclera, sclera, uveal tract, retina, vasculature, optic nerve, and orbit Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. Uveitis can arise from a number of different causes and is associated with a number of different diseases, including, but not limited to, rheumatic diseases such as rheumatic diseases (e.g., ankylosing spondylitis and juvenile rheumatoid arthritis), certain infectious diseases such as tuberculosis and syphilis, other conditions such as sarcoidosis, systemic lupus erythematosus, chemical injury, trauma, surgery, etc. In some embodiments, the type of uveitis is anterior uveitis. In some embodiments, the type of uveitis is posterior uveitis. Keratis refers to inflammation of the cornea. Keratitis has a diverse array of causes including bacterial, viral, or fungal infection, trauma, and allergic reaction. Amoebic infection of the cornea, e.g., caused by Acanthamoeba, is a particular problem for contact lens wearers. Scleritis refers to inflammation of the sclera. Uveitis, keratitis, and scleritis, and methods for their diagnosis are well known in the art. Symptoms of the various inflammatory conditions that affect the eye can include, but are not limited to, eye pain, redness, light sensitivity, tearing, blurred vision, floaters. Ocular inflammation of various types is well known to occur in association with a variety of local or systemic diseases, some of which are noted above. In some instances, the cause may remain unknown.

Dry AMD is characterized by the existence of deposits known as drusen and the separation of the RPE from BM, which is often accompanied by RPE atrophy and apoptosis and loss of underlying choriocapillaris and overlying photoreceptors, resulting in some instances in areas of geographic atrophy which can eventually coalesce to form large patches. In exudative AMD, new blood vessels grow from the choriocapillaris through Bruch's membrane and can extend into the RPE and photoreceptor cell layers (choroidal neovascularization). These blood vessels can bleed and leak fluid, frequently resulting in sudden visual loss due to events such as RPE and/or retinal detachment. Eventually a fibrovascular scar may form, leading to irreversible visual loss. In some forms of neovascular AMD, angiomatous proliferation originates from the retina and extends posteriorly into the subretinal space, eventually communicating in some cases with new choroidal vessels. This form of neovascular AMD, termed retinal angiomatous proliferation (RAP), can be particularly severe. It has been suggested that angiomatous proliferation within the retina is the first manifestation of the vasogenic process in this form of neovascular AMD. Dilated retinal vessels and pre-, intra-, and subretinal hemorrhages and exudate evolve, surrounding the angiomatous proliferation as the process extends into the deep retina and subretinal space.

The present disclosure provides compositions (e.g. microbiome compositions) and methods that inhibit one or more of the events or processes that take place in AMD. The present disclosure is based in part on the discovery that one or more microbial strains are particularly suitable as therapeutic agents for macular degeneration and related conditions, for diabetic retinopathy, and/or for choroidal neovascularization associated with any of these disorders, or others.

Microbial Preparation(s) and/or Component(s)

The present disclosure provides systems and methods for assessing, characterizing, and identifying one or more microbial strains of a microbiome. For example, the present disclosure provides systems and methods for assessing, characterizing, and identifying one or more microbial strains of a microbiome that have one or more abilities. Such systems and methods can be useful for assessing, characterizing, and identifying one or more microbial strains that affect the health of humans, livestock, and/or pets. In some embodiments, one or more microbial strains affect the health of humans, livestock, and/or pets by modulating their respective metabolomes, oxidative stress, one or more parameters or features (e.g. of an organ of a subject), or a combination thereof to prevent, treat, or reduce the risk of suffering from a disease, disorder, or condition. For example, technologies described herein may result in modulating the metabolome, reduce oxidative stress, one or more parameters or features, or a combination thereof of the subject that results in a decrease in production of toxic components (e.g. drusen) in a subject (e.g. in an eye of a subject).

The present disclosure also provides systems and methods for manufacturing a pharmaceutical composition that comprise assessing, characterizing, and identifying one or more microbial strains of a microbiome.

In some embodiments, assessing, characterizing, and identifying one or more microbial strains from a microbiome of a snake, lizard, fish, or bird. In some embodiments, assessing, characterizing, and identifying one or more microbial strains from a mammalian microbiome. A mammalian microbiome can be a canine, a feline, an equine, a bovine, an ovine, a caprine, or a porcine microbiome. In some embodiments, a microbiome used in a system or method described herein may prevent or treat a disease or condition.

A microbiome can be isolated from any system or tissue of an organism that supports microbial growth. For example, a microbiome can be a cutaneous microbiome, an oral microbiome, a nasal microbiome, a gastrointestinal microbiome, a brain microbiome, a pulmonary microbiome, or a urogenital microbiome. A list of exemplary microbial strains found in a gastrointestinal microbiome is included below in Table 1. A person skilled in the art would understand that a microbiome sample can be obtained by various ways known in the art. For example, a cutaneous, oral, nasal, pulmonary, or urogenital microbiome sample could be obtained using a swab or tissue scrapping. In some embodiments, a gastrointestinal microbiome could be sampled from feces. A cutaneous microbiome, an oral microbiome, a nasal microbiome, a gastrointestinal microbiome, a brain microbiome, a pulmonary microbiome, or a urogenital microbiome sample could be obtained via a biopsy.

In some embodiments, a microbiome is a microbiome of a healthy individual or an individual who does not suffer from or is not at risk of developing a particular disease or disorder. In some embodiments, a microbiome is a microbiome of an individual that suffers from or is at risk of developing a particular disease or disorder. In some embodiments, a microbiome is a microbiome of an individual who is known to suffer from a particular disease or disorder. In some embodiments, a human microbiome is a microbiome of a human with an unknown risk for one or more diseases or conditions.

In some embodiments, a microbiome is a reference microbiome. A reference microbiome can be a microbiome of a healthy individual or an individual who does not suffer from or is not at risk of developing a particular disease or disorder. In some instances, a reference microbiome may be from the same individual as a microbiome to be assessed or characterized, but was obtained at a different time. In some instances, a reference microbiome may be from the same individual as a microbiome to be assessed or characterized, but was obtained from a different system or tissue.

In some embodiments, an individual microbial strain or a combination of microbial strains may be assessed, characterized, or identified in a different relative amount than such strain or strains are found in a microbiome. For example, the effect of modulation of a cell or organism in response to a single strain may be assessed, characterized, or identified using in vitro methods (e.g. mammalian cells) or in vivo methods using mammals (e.g. mice, humans, etc.) as described herein. In some embodiments, for example, the effect of modulation of a cell or organism to treat, prevent, or reduce the risk on a disease, disorder, or condition (e.g. an ocular disease, disorder, or condition as described herein) may be assessed, characterized, or identified using in vitro methods (e.g. mammalian cells) or in vivo methods using mammals (e.g. mice, humans, etc.) as described herein. In some embodiments, for example, the effect of modulation of a cell or organism to treat, prevent, or reduce the risk on a disease, disorder, or condition (e.g. an ocular disease, disorder, or condition as described herein) by modulating one or more metabolites of the cell or organism, one or features or parameters (e.g. cell viability, size/amount of drusen, level or activity of a nucleic acid or protein, or form thereof, etc.) of the cell or organism, or a combination thereof may be assessed, characterized, or identified using in vitro methods (e.g. mammalian cells) or in vivo methods using mammals (e.g. mice, humans, etc.) as described herein. As another example, the effect of modulation (e.g. of levels of one or more metabolites) of a cell or organism to treat, prevent, or reduce the risk on a disease, disorder, or condition, as described herein, in response to two microbial strains may be assessed, characterized, or identified together using methods described herein.

An extract, component, or compound of a microbial strain may also be assessed, characterized, or identified using methods described herein. In some cases, an extract, component, or compound of a microbial strain that has been determined to treat, prevent, or reduce the risk on a disease, disorder, or condition, as described herein, in an organism (e.g. mammal) may be assessed, characterized, or identified. Assessing, characterizing or identifying an extract, component, or compound of a microbial strain that treats, prevents, or reduces the risk on a disease, disorder, or condition in an organism (e.g. mammal) may provide additional information about potential biomarkers, targets, or protective agents in a microbiome.

A variety of technologies are known in the art that can be used to prepare extracts of microbial strains, and/or to isolate extracts, components, or compounds therefrom, or to process (e.g., to isolate and/or purify one or more components or compounds from). To give but a few examples, such technologies may include, for example, one or more of organic extraction, vacuum concentration, chromatography, and so on.

Assessing Biological Impact

The present disclosure provides the insight that compositions (e.g. microbiome compositions) as described herein can be used to treat, prevent, and/or reduce the risk of a disease, disorder, or condition of an organism (e.g. a mammal (e.g. a human)) by contacting the composition(s) (e.g., feeding the compositions to, administering to) with an organism. In some embodiments, an organism may suffer from or be at risk of suffering from a disease, disorder, or condition (e.g. mammalian disease, disorder, or condition). To determine whether one or more compositions treats, prevents, or reduces the risk of a disease, disorder, or condition (e.g. an ocular disease, disorder, or condition), levels of one or more metabolites can be observed, measured, or assessed in samples that have been contacted with the one or more compositions. For example, levels of the one or more metabolites can be observed, measured, or assessed in samples at different times (e.g. before administration of composition, after administration of composition, during administration of composition, etc.). To determine whether one or more compositions treats, prevents, or reduces the risk of a disease, disorder, or condition (e.g. an ocular disease, disorder, or condition), one or more features or parameters may be observed, measured, or assessed in samples that have been contacted with the one or more compositions. For example, one or more features or parameters may be observed, measured, or assessed in samples at different times (e.g. before administration of composition, after administration of composition, during administration of composition, etc.).

In some embodiments, methods described herein utilize a first sample and a second sample. In some embodiments, a first sample is a reference sample. In some embodiments, a reference sample can be a sample obtained from a subject who is contacted with (e.g., administered or fed) a composition, e.g., CT10 composition or CT6 composition. In some embodiments, a reference sample can be a sample obtained from a subject who is contacted with (e.g., administered or fed) a composition, e.g., CT10 composition or CT6 composition, at a first time point. In some embodiments, a reference sample can be a sample obtained from a subject prior to being contacted with (e.g., administered or fed) a composition, e.g., CT10 composition or CT6 composition. In some embodiments, a reference sample can be a sample obtained from a healthy individual. In some embodiments, a reference sample can be a sample obtained from an individual who is suffering from or may have a risk for a disease, disorder, or condition (e.g. ocular disease, disorder, or condition). In some embodiments, a reference sample is a control sample. In some embodiments, a reference sample is a negative control sample. In some embodiments, a reference sample is a positive control sample. In some embodiments, a reference sample may be a historic reference (e.g. value across control samples). In some embodiments, a reference sample may be from a printed publication (e.g. a text book, a journal, etc.).

In some embodiments, a second sample can be a test sample. In some embodiments, a test sample may be a sample obtained from a subject who is contacted with (e.g., administered or fed) a composition, e.g., CT10 composition or CT6 composition. In some instances, a subject (e.g. patient or population) may be suffering from or at risk of a disease, disorder, or condition (e.g. ocular disease, disorder, or condition). In some instances, a subject (e.g. patient or population) may have an unknown risk for one or more diseases, disorders, or conditions as described herein. In some embodiments, a test can be a sample obtained from a subject who is contacted with (e.g., administered or fed) a composition, e.g., CT10 composition or CT6 composition, at a second time point.

In some embodiments, methods described herein comprise comparing one or more metabolite levels (e.g. a metabolome), or one or more parameters or features (e.g. cell viability, size/amount of drusen, level or activity of a nucleic acid or protein, or form thereof, etc.) obtained from a test sample with one or more metabolite levels (e.g. a metabolome), or one or more parameters or features (e.g. cell viability, size/amount of drusen, level or activity of a nucleic acid or protein, or form thereof, etc.) obtained from a reference sample. In some embodiments, by comparing one or more metabolite levels, parameters, or features obtained from a test sample with one or more metabolite levels, parameters, or features obtained from a reference sample, a composition described herein can be assessed, characterized or identified as being useful for treating, preventing, or reducing the risk of suffering from a disease, disorder, or condition (e.g. ocular disease, disorder, or condition) as described herein. In some embodiments, by comparing one or more metabolite levels, parameters, or features obtained from a test sample with one or more metabolite levels, parameters, or features obtained from a reference sample, it can be determined that a composition as disclosed herein increases the severity or incidence of a disease, disorder, or condition phenotype. In some embodiments, by comparing one or more metabolite levels, parameters, or features obtained from a test sample with one or more metabolite levels, parameters, or features obtained from a reference sample, it can be determined that a composition as disclosed herein decreases the severity or incidence of a disease, disorder, or condition phenotype. In some embodiments, by comparing one or more metabolite levels, parameters, or features obtained from a test sample with one or more metabolite levels, parameters, or features obtained from a reference sample, it can be determined that a composition as disclosed herein has no effect on the severity or incidence of a disease, disorder, or condition phenotype. In some embodiments, by comparing one or more metabolite levels, parameters, or features obtained from a test sample with one or more metabolite levels, parameters, or features obtained from a reference sample, it can be determined that a composition as disclosed herein prevents a disease, disorder, or condition phenotype.

The present disclosure also provides the recognition that compositions and methods provided herein can be used to monitor progression of a disease, disorder, or condition (e.g. ocular disease, disorder, or condition) in an individual. For example, if metabolite levels, parameters or features (e.g. cell viability, size/amount of drusen, level or activity of a nucleic acid or protein, or form thereof, etc.) determined to increase the severity of a disease, disorder, or condition decrease in relative amount, it may indicate that the disease, disorder, or condition is being attenuated, e.g., by treatment or immune response.

The present disclosure also provides the insight that compositions and methods provided herein can be used to tailor treatments (e.g., therapies, nutraceuticals, and/or probiotics) to an individual patient. In some embodiments, compositions and methods provided herein can provide “personalized” therapy. In some cases, metabolite levels, features or parameters (e.g. cell viability, size/amount of drusen, level or activity of a nucleic acid or protein, or form thereof etc.) within an individual can be assessed, characterized, or identified to determine if they have a disease, disorder, or condition. Based on the results, the individual can be treated with one or more compositions to adjust the metabolite levels (i.e., their metabolome), features or parameters. In some instances, this will affect the disease, disorder, or condition the individual is suffering from or at risk of developing. For example, if an individual is determined to have a relatively low amount of one or more metabolite levels that have been determined to decrease the severity of a disease, disorder, or condition, administration of the one or more compositions that have been determined to decrease the severity of a disease, disorder, or condition to the individual (or an extract, component, or compound thereof) may attenuate the severity of the individual's disease or condition.

The present disclosure provides the insight that compositions and methods provided herein can be used recursively to treat, prevent, or ameliorate a disease, disorder, or condition. In some embodiments, for example, one or more compositions disclosed herein may be administered (e.g. fed, injected, etc.) to a subject after determining the effect of one or more compositions on subject's metabolite levels, or after determining the effect of one or more compositions on subject's features or parameters (e.g. cell viability, size/amount of drusen, level or activity of a nucleic acid or protein, or form thereof, etc). In some embodiments, a composition may be administered once. In some embodiments, a composition may be administered more than once. In some embodiments, a composition may be administered daily, weekly, biweekly, monthly, bimonthly, etc. In each of these instances, levels of one or more metabolites, or changes in features or parameters may be monitored. In some embodiments, levels of one or more metabolites (e.g. metabolome) or changes in features or parameters may be monitored before administration of a composition. In some embodiments, levels of one or more metabolites (e.g. metabolome) or changes in features or parameters may be monitored after administration of a composition.

Pharmaceutical Compositions

Provided herein are compositions comprising individual microbial strains or combinations of microbial strains, metabolites thereof, extracts thereof, or components thereof. In some embodiments, a composition comprises individual microbial strains or combinations of microbial strains from a mammalian microbiome, metabolites thereof, extracts thereof, and/or components thereof, which have been assessed, identified, characterized or assayed using methods as described herein. In some embodiments, a composition provided herein comprises one or more, two or more, three or more, four or more, five or more, six or more, seven or more, eight or more, nine or more, or ten or more microbial strains from a mammalian microbiome, extracts thereof, metabolites thereof, and/or components thereof, which have been assessed, identified, characterized or assayed using methods as described herein.

Provided herein are also compositions comprising one or more components or metabolites. In some embodiments, components or metabolites in compositions herein are from a source that is not a microbial strain, e.g., synthetically generated. In some embodiments, components or metabolites in a composition may have been identified from a microbial strain, but are independent from a microbial strain and are not produced by a microbial strain, e.g., they can be synthetically generated.

In some embodiments, a composition provided herein comprises two or more, three or more, four or more, five or more, six or more, seven or more, eight or more, nine or more, or ten or more microbial strains listed in Table 1 below.

TABLE 1
Table 1: Exemplary Microbial Strains Found in Human Gut Microbiome

| | |
|---|---|
| Bacteroides pectinophilus | Exiguobacterium mexicanum |
| Acetobacter sp | Faecalibacterium prausnitzii |
| Acetobacterium tundrae | Faecalitalea cylindroides |
| Achromobacter aegrifaciens | Finegoldia magna |
| Achromobacter insuavis | Flavonifractor plautii |
| Achromobacter piechaudii | Flintibacter butyricus |
| Achromobacter xylosoxidans | Fusicatenibacter saccharivorans |
| Acidaminococcus fermentans | Fusobacterium gonidiaformans |
| Acidaminococcus intestini | Fusobacterium mortiferum |
| Acinetobacter baumannii | Fusobacterium nucleatum |
| Acinetobacter junii | Fusobacterium ulcerans |
| Actinomyces sp. | Fusobacterium varium |
| Agathobacter rectalis | Gardnerella vaginalis |
| Agathobaculum butyriciproducens | Gemella haemolysans |
| Aggregatibacter segnis | Gemella sanguinis |
| Akkermansia muciniphila | Gemmiger formicilis |
| Alistipes finegoldii | Gluconacetobacter sp |
| Alistipes indistinctus | Gluconobacter sp |
| Alistipes onderdonkii | Gordonibacter pamelaeae |
| Alistipes putredinis | Granulicatella adiacens |
| Alistipes shahii | Grimontia hollisae |
| Allisonella histaminiformans | Haemophilus parainfluenzae |
| Anaerobaculum hydrogeniformans | Harryflintia acetispora |
| Anaerococcus hydrogenalis | Helicobacter bilis |
| Anaerococcus octavius | Helicobacter bizzozeronii |
| Anaerococcus prevotii | Helicobacter canadensis |
| Anaerococcus tetradius | Helicobacter cinaedi |
| Anaerococcus vaginalis | Helicobacter pullorum |
| Anaerofilum agile | Helicobacter pylori |
| Anaerofustis stercorihominis | Helicobacter winghamensis |
| Anaerosporobacter mobilis | Holdemanella biformis |
| Anaerostipes caccae | Holdemania filiformis |
| Anaerostipes hadrus | Holdemania massiliensis |
| Anaerostipes rhamnosivorans | Hungatella effluvii |
| Anaerotruncus colihominis | Hungatella hathewayi |
| Anaerovorax odorimutans | Intestinimonas butyriciproducens |
| Arcobacter butzleri | Kineothrix alysoides |
| Asaccharobacter celatus | Kingella oralis |
| Atopobium parvulum | Klebsiella pneumoniae |
| Atopobium vaginae | Klebsiella pneumoniae subsp. ozaenae |
| Bacillus cereus | Klebsiella pneumoniae subsp. pneumoniae |
| Bacillus coagulans | Klebsiella pneumoniae subsp. rhinoscleromatis |
| Bacillus licheniformis | Klebsiella quasipneumoniae subsp. quasipneumoniae |
| Bacillus pseudomycoides | Klebsiella singaporensis |
| Bacillus sonorensis | Klebsiella variicola |
| Bacillus toyonensis | Lachnobacterium bovis |
| Bacillus wiedmannii | Lachnospira multipara |
| Bacteroides caccae | Lachnospira pectinoschiza |
| Bacteroides cellulosilyticus | Lactobacillus acidophilus |
| Bacteroides clarus | Lactobacillus amylolyticus |
| Bacteroides coprocola | Lactobacillus amylovorus |
| Bacteroides coprophilus | Lactobacillus antri |
| Bacteroides dorei | Lactobacillus brevis subsp. Gravesensis |
| Bacteroides eggerthii | Lactobacillus buchneri |
| Bacteroides faecis | Lactobacillus casei |
| Bacteroides finegoldii | Lactobacillus coryniformis subsp. Coryniformis |
| Bacteroides fluxus | Lactobacillus crispatus |
| Bacteroides fragilis | Lactobacillus delbrueckii subsp. Bulgaricus |
| Bacteroides intestinalis | Lactobacillus delbrueckii subsp. indicus |
| Bacteroides massiliensis | Lactobacillus delbrueckii subsp. Lactis |
| Bacteroides nordii | Lactobacillus fermentum |
| Bacteroides oleiciplenus | Lactobacillus fructivorans |
| Bacteroides ovatus | Lactobacillus gasseri |
| Bacteroides plebeius | Lactobacillus helveticus |
| Bacteroides salanitronis | Lactobacillus hilgardii |
| Bacteroides salyersiae | Lactobacillus iners |
| Bacteroides stercoris | Lactobacillus jensenii |
| Bacteroides thetaiotaomicron | Lactobacillus johnsonii |
| Bacteroides uniformis | Lactobacillus mucosae |
| Bacteroides vulgatus | Lactobacillus oris |
| Bacteroides xylanisolvens | Lactobacillus paracasei |
| Bacteroides xylanolyticus | Lactobacillus paracasei subsp. tolerans |
| Barnesiella intestinihominis | Lactobacillus pentosus |
| Bartonella clarridgeiae | Lactobacillus plantarum subsp. plantarum |
| Bartonella quintana str. Toulouse | Lactobacillus reuteri |
| Bifidobacterium adolescentis | Lactobacillus rhamnosus |
| Bifidobacterium angulatum | Lactobacillus rogosae |
| Bifidobacterium animalis | Lactobacillus ruminis |
| Bifidobacterium bifidum | Lactobacillus salivarius |
| Bifidobacterium breve | Lactobacillus ultunensis |
| Bifidobacterium catenulatum | Lactobacillus vaginalis |
| Bifidobacterium coryneforme | Lactococcus formosensis |
| Bifidobacterium dentium | Lactococcus garvieae |
| Bifidobacterium faecale | Lactococcus lactis subsp. Cremoris |
| Bifidobacterium gallicum | Lactococcus lactis subsp. lactis |
| Bifidobacterium longum | Lactonifactor longoviformis |
| Bifidobacterium longum subsp. infantis | Laribacter hongkongensis |
| Bifidobacterium longum subsp. longum | Lautropia mirabilis |
| Bifidobacterium longum subsp. suis | Leptotrichia buccalis |
| Bifidobacterium pseudocatenulatum | Leptotrichia hofstadii |
| Bifidobacterium pseudolongum | Leuconostoc lactis |
| Bifidobacterium stercoris | Leuconostoc mesenteroides subsp. Cremoris |
| Bilophila wadsworthia | Listeria grayi |
| Bittarella massiliensis | Listeria monocytogenes |
| Blautia coccoides | Longicatena caecimuris |
| Blautia faecis | Marvinbryantia formatexigens |
| Blautia glucerasea | Megamonas funiformis |
| Blautia hansenii | Megamonas rupellensis |
| Blautia hydrogenotrophica | Megasphaera elsdenii |
| Blautia luti | Megasphaera indica |
| Blautia obeum | Megasphaera micronuciformis |
| Blautia producta | Megasphaera paucivorans |
| Blautia schinkii | Methanobrevibacter smithii |
| Blautia stercoris | Methanomassiliicoccus luminyensis |
| Blautia wexlerae | Methanosphaera stadtmanae |
| Bradyrhizobium japonicum | Methylobacterium radiotolerans |
| Burkholderia ambifaria | Mitsuokella jalaludinii |
| Burkholderia cenocepacia | Mitsuokella multacida |
| Burkholderia glumae | Mobiluncus mulieris |
| Burkholderia multivorans | Mogibacterium timidum |
| Burkholderia plantarii | Mogibacterium vescum |
| Butyricicoccus faecihominis | Moraxella catarrhalis |
| Butyricicoccus pullicaecorum | Morganella morganii subsp. morganii |
| Butyricimonas faecihominis | Murdochiella asaccharolytica |
| Butyricimonas paravirosa | Mycobacterium abscessus |
| Butyricimonas virosa | Mycobacterium tuberculosis |
| Butyrivibrio crossotus | Mycoplasma hominis |
| Campylobacter coli | Neisseria cinerea |
| Campylobacter concisus | Neisseria flavescens |
| Campylobacter curvus | Neisseria macacae |
| Campylobacter gracilis | Neisseria mucosa |
| Campylobacter hominis | Neisseria sicca |
| Campylobacter jejuni subsp. Jejuni | Neisseria subflava |
| Campylobacter showae | Nitrobacter hamburgensis |
| Campylobacter upsaliensis | Nitrobacter winogradskyi |
| Candidatus Dorea massiliensis | Odoribacter laneus |
| Candidatus Stoquefichus massiliensis | Odoribacter splanchnicus |
| Capnocytophaga gingivalis | Olsenella profusa |
| Capnocytophaga sputigena | Olsenella scatoligenes |
| Cardiobacterium hominis | Olsenella uli |
| Catenibacterium mitsuokai | Oribacterium sinus |
| Catonella morbi | Oscillibacter ruminantium |
| Cedecea lapagei | Oscillibacter valericigenes |
| Citrobacter amalonaticus | Oscillospira guilliermondii |
| Citrobacter freundii | Oxalobacter formigenes |
| Citrobacter koseri | Paenibacillus jamilae |
| Citrobacter youngae | Paenibacillus kribbensis |
| Clostridium acetobutryicum | Paenibacillus riograndensis |
| Clostridium aerotolerans | Paeniclostridium sordellii |
| Clostridium aldenense | Parabacteroides distasonis |
| Clostridium aminophilum | Parabacteroides goldsteinii |
| Clostridium aminovalericum | Parabacteroides gordonii |
| Clostridium amygdalinum | Parabacteroides johnsonii |
| Clostridium asparagiforme | Parabacteroides merdae |
| Clostridium baratii | Paraprevotella clara |
| Clostridium bartlettii | Paraprevotella xylaniphila |
| Clostridium beijerinckii | Parasutterella excrementihominis |
| Clostridium bifermentans | Parasutterella secunda |
| Clostridium bolteae | Parvimonas micra |
| Clostridium butyricum | Pediococcus acidilactici |
| Clostridium celerecrescens | Pediococcus pentosaceus |
| Clostridium cf. saccharolyticum | Peptoniphilus duerdenii |
| Clostridium citroniae | Peptoniphilus grossensis |
| Clostridium clariflavum | Peptoniphilus harei |
| Clostridium clostridioforme | Peptoniphilus indolicus |
| Clostridium cocleatum | Peptostreptococcus anaerobius |
| Clostridium colinum | Phascolarctobacterium faecium |
| Clostridium difficile | Phascolarctobacterium succinatutens |
| Clostridium glycyrrhizinilyticum | Porphyromonas asaccharolytica |
| Clostridium hathewayi | Porphyromonas endodontalis |
| Clostridium herbivorans | Porphyromonas gingivalis |
| Clostridium hiranonis | Prevotella bivia |
| Clostridium hylemonde | Prevotella buccae |
| Clostridium innocuum | Prevotella copri |
| Clostridium lactatifermentans | Prevotella disiens |
| Clostridium lavalense | Prevotella marshii |
| Clostridium leptum | Prevotella melaninogenica |
| Clostridium methoxybenzovorans | Prevotella nigrescens |
| Clostridium methylpentosum | Prevotella pallens |
| Clostridium nexile | Prevotella salivae |
| Clostridium orbiscindens | Prevotella stercorea |
| Clostridium oroticum | Prevotella tannerae |
| Clostridium perfringens | Prevotella timonensis |
| Clostridium polysaccharolyticum | Propionibacterium acnes |
| Clostridium propionicum | Propionibacterium avidum |
| Clostridium ramosum | Propionibacterium namnetense |
| Clostridium rectum | Proteus mirabilis |
| Clostridium saccharogumia | Proteus penneri |
| Clostridium saccharolyticum | Providencia alcalifaciens |
| Clostridium sardiniense | Providencia rettgeri |
| Clostridium saudii | Providencia rustigianii |
| Clostridium scindens | Providencia stuartii |
| Clostridium sordellii | Pseudoflavonifractor capillosus |
| Clostridium sphenoides | Ralstonia sp. |
| Clostridium spiroforme | Robinsoniella peoriensis |
| Clostridium sporogenes | Roseburia cecicola |
| Clostridium sticklandii | Roseburia faecis |
| Clostridium straminisolvens | Roseburia hominis |
| Clostridium symbiosum | Roseburia intestinalis |
| Clostridium tertium | Roseburia inulinivorans |
| Clostridium thermocellum | Rothia dentocariosa |
| Clostridium xylanolyticum | Ruminococcus albus |
| Clostridium xylanovorans | Ruminococcus bromii |
| Collinsella aerofaciens | Ruminococcus callidus |
| Collinsella intestinalis | Ruminococcus faecis |
| Collinsella stercoris | Ruminococcus gnavus |
| Collinsella tanakaei | Ruminococcus lactaris |
| Coprobacillus cateniformis | Ruminococcus obeum |
| Coprobacter fastidiosus | Ruminococcus torques |
| Coprococcus catus | Ruthenibacterium lactatiformans |
| Coprococcus comes | Sarcina ventriculi |
| Coprococcus eutactus | Sellimonas intestinalis |
| Corynebacterium ammoniagenes | Senegalimassilia anaerobia |
| Corynebacterium matruchotii | Shigella boydii |
| Corynebacterium pseudogenitalium | Shigella dysenteriae |
| Corynebacterium tuberculostearicum | Shigella flexneri |
| Deinococcus radiodurans | Shigella sonnei |
| Dermabacter hominis | Slackia faecicanis |
| Desulfotomaculum guttoideum | Slackia isoflavoniconvertens |
| Desulfovibrio legallis | Slackia piriformis |
| Desulfovibrio piger | Solobacterium moorei |
| Dialister invisus | Staphylococcus caprae |
| Dialister microaerophilus | Staphylococcus epidermidis |
| Dialister succinatiphilus | Staphylococcus hominis subsp. Hominis |
| Dielma fastidiosa | Staphylococcus lugdunensis |
| Dorea formicigenerans | Staphylococcus warneri |
| Dorea longicatena | Streptococcus agalactiae |
| Dysgonomonas mossii | Streptococcus anginosus |
| Edwardsiella tarda | Streptococcus anginosus subsp. whileyi |
| Eggerthella lenta | Streptococcus australis |
| Eggerthella sinensis | Streptococcus bovis |
| Eikenella corrodens | Streptococcus constellatus subsp. constellatus |
| Eisenbergiella tayi | Streptococcus equinus |
| Enhydrobacter aerosaccus | Streptococcus gallolyticus subsp. pasteuri |
| Enterobacter aerogenes | Streptococcus gallolyticus subsp. pasteurianus |
| Enterobacter asburiae | Streptococcus gordonii |
| Enterobacter cancerogenus | Streptococcus gordonii str. Challis |
| Enterobacter cloacae | Streptococcus infantarius |
| Enterobacter hormaechei | Streptococcus infantarius subsp. coli |
| Enterobacter kobei | Streptococcus infantarius subsp. Infantarius |
| Enterobacter ludwigii | Streptococcus infantis |
| Enterobacter xiangfangensis | Streptococcus lactarius |
| Enterococcus asini | Streptococcus lutetiensis |
| Enterococcus avium | Streptococcus mutans |
| Enterococcus casseliflavus | Streptococcus parasanguinis |
| Enterococcus durans | Streptococcus pasteurianus |
| Enterococcus faecalis | Streptococcus pleomorphus |
| Enterococcus faecium | Streptococcus rubneri |
| Enterococcus gallinarum | Streptococcus salivarius |
| Enterococcus hirae | Streptococcus salivarius subsp. salivarius |
| Enterococcus mundtii | Streptococcus sanguinis |
| Enterococcus raffinosus | Streptococcus thermophilus |
| Enterococcus raffinosus | Streptococcus vestibularis |
| Erysipelotrichaceae bacterium | Subdoligranulum variabile |
| Escherichia albertii | Succinatimonas hippei |
| Escherichia coli | Sutterella parvirubra |
| Escherichia fergusonii | Sutterella stercoricanis |
| Eubacterium biforme | Sutterella wadsworthensis |
| Eubacterium callanderi | Terrisporobacter glycolicus |
| Eubacterium contortum | Turicibacter sanguinis |
| Eubacterium cylindroides | Ureaplasma parvum |
| Eubacterium desmolans | Vagococcus penaei |
| Eubacterium dolichum | Varibaculum cambriense |
| Eubacterium eligens | Veillonella sp. |
| Eubacterium hadrum | Veillonella dispar |
| Eubacterium hallii | Veillonella parvula |
| Eubacterium infirmum | Veillonella rogosae |
| Eubacterium limosum | Veillonella tobetsuensis |
| Eubacterium oxidoreducens | Vibrio cholerae |
| Eubacterium ramulus | Vibrio furnissii |
| Eubacterium rectale | Vibrio mimicus |
| Eubacterium ruminantium | Victivallis vadensis |
| Eubacterium saburreum | Weissella cibaria |
| Eubacterium siraeum | Weissella confusa |
| Eubacterium sulci | Weissella paramesenteroides |
| Eubacterium tortuosum | Xenorhabdus nematophila |
| Eubacterium ventriosum | Yersinia enterocolitica subsp. Palearctica |
| Eubacterium xylanophilum | Yersinia pseudotuberculosis |
| Eubacterium yurii subsp. Margaretiae | |

In some embodiments, a composition provided herein comprises Gluconacetobacter hansenii, Terrisporobacter glycolicus, Coprococcus sp., Lactobacillus plantarum, Clostridium butyricwn, Paenibacillus sp., Veillonella sp., Bifidobacterium, Bacillus subtilis, Acidaminococcus sp., or a combination thereof. In some embodiments, a composition comprises at least two of, at least three of, at least four of, at least five of, at least six of, at least seven of, at least eight of, at least nine of, or all of Gluconacetobacter hansenii, Terrisporobacter glycolicus, Coprococcus sp., Lactobacillus plantarum, Clostridium butyricum, Paenibacillus sp., Veillonella sp., Bifidobacterium, Bacillus subtilis, and Acidaminococcus sp. In some embodiments, for example, a composition comprises all of Gluconacetobacter hansenii, Terrisporobacter glycolicus, Coprococcus sp., Lactobacillus plantarum, Clostridium butyricwn, Paenibacillus sp., Veillonella sp., Bifidobacterium sp., Bacillus subtilis, and Acidaminococcus sp., and may be referred to by different names, including but not limited to, CT10 composition, CT10 cocktail, and so forth.

In some embodiments, a composition provided herein comprises Gluconacetobacter hanseni, Terrisporobacter glycolicus, Coprococcus sp., Lactobacillus plantarum, Veillonella atypica, Bifidobacterium, or a combination thereof. In some embodiments, a composition comprises at least two of, at least three of, at least four of at least five of, or all of Gluconacetobacter hanseni, Terrisporobacter glycolicus. Coprococcus sp., Lactobacillus plantarum, Veillonella atypica, and Bifidobacterium. In some embodiments, for example, a composition comprises all of Gluconacetobacter hanseni, Terrisporobacter glycolicus, Coprococcus sp., Lactobacillus plantarum, Veillonella atypica, and Bifidobacterium and may be referred to by different names, including but not limited to, CT6 composition, CT6 cocktail, and so forth.

In some embodiments, a composition provided herein comprises one or more, two or more, three or more, four or more, five or more, six or more, seven or more, eight or more, nine or more, or ten or more metabolites. Metabolites which may be assessed, identified, characterized, or assayed and/or comprised in compositions as disclosed herein, include those listed for example in the Appendix submitted herewith (e.g. Appendix 1-1, 1-2, 2, or 3).

In some embodiments, a metabolite may be Butyrylcamitine, Theobromine, p-Hydroxyphenylpyruvic acid, Propionic acid. Picolinic acid, 2-Hydroxy-4methylvaleric acid, N6-Acetylysine, Urocanic acid, N5-Ethylglutamine, Trigonelline, Stachydrine, Ectoine, 5-Hydroxylysine, Arginine (arg), Cholic acid, 2(4-Hydroxyphenyl)propionic acid, N-Acetyltryptophan, Hydroxyproline, Argininosuccinic acid, Glutamic acid (Glu), Sarcosine, 5-Methoxyindoleacetic acid, Indole-3-lactic acid, Isovalerylalanine, N-Acetylleucine, 1-Methylhistidine, N-Acetylephenylalanine, Proline (Pro), or any combination thereof.

In some embodiments, a metabolite may be 4-Hydroxyphenylpyruvic. Ectoine, Gramine, N-Acetyl-L-phenylalanine, Nepsilon-Acetyl-L-lysine, Stachydrine, Trigonelline, 3-Ureidopropionic acid, Theobromine, Hippuric acid, Imidazolepropionic acid, NG-Methyl-L-arginine, trans-Urocanic Acid, N-Acetyl-L-leucine, Sarcosine, Isobutyrylcamitine, b-Hydroxyisovaleric acid, L-Theanine/N5-Ethylglutamine, 5-Hydroxylysine, Phenaceturic acid, betaine, hydroxyproline, Picolinic acid, 2-Aminoadipic acid, Glycerophosphocholine, camitine, Glycerol 3-phosphate, Argininosuccinic acid, creatine, Terephthalic acid. Homocitrulline. Mucic acid, Homocysteinesulfinic acid, Trimethyllysine, Spermidine, Glyoxylic acid, XA0013 C6H604S, 3-Indoxylsulfuric acid, Nicotinamide, N-Formylglycine, Ureidoglycolate, N-Methylproline, Glucaric acid, Butyrylcarnitine, Methionine sulfoxide, Carboxymethyllysine, Glycolic acid, Phenaceturic acid, Diethanolamnne, Phosphorylcholine, Guanidinosuccinic acid, N-Acetylhistidine, Glyceric acid, S-Methylmethionine, Cysteine glutathione disulfide, Kynurenine, N-Acetylphenylalanine, Threonic acid, Malic acid, 7,8-Dihydrobiopterin, Homovanillic acid, Taurocholic acid, 5-Methoxyindoleacetic acid, butyrate, b-Hydroxyisovaleric acid, 2-Oxoglutaric acid, N-Acetyltryptophan, Thiaproline, Hypotaurine, Cholic acid, Acetoacetic acid, Ethanolamine, Guanidoacetic acid, S-Sulfocysteine, Myristic acid C14:0 XA0027, or any combination thereof.

In some embodiments, an individual microbial strain or combinations of microbial strains from a mammalian microbiome that have been killed (e.g., heat killed). Alternatively, in some embodiments, an individual microbial strain or combinations of microbial strains from a mammalian microbiome may include cells that are viable or alive.

In some embodiments, one or more microbial strains comprise a viable or living individual microbial strain or combinations of microbial strains, e.g., from a mammalian microbiome.

In some embodiments, one or more microbial strains comprise a viable or living individual microbial strain or combinations of microbial strains, e.g., from a mammalian microbiome, as described herein comprises and/or is formulated through use of one or more cell cultures and/or supernatants or pellets thereof, and/or a powder formed therefrom.

In some embodiments, compositions for use in accordance with the present disclosure are pharmaceutical compositions, e.g., for administration (e.g., oral administration, ophthalmic administration, intravitreal administration, or suprachoroidal administration) to a mammal (e.g., a human). Pharmaceutical compositions typically include an active agent (e.g., individual microbial strains or combinations of microbial strains from a mammalian microbiome, extracts thereof, and/or components thereof), and a pharmaceutically acceptable carrier. Certain exemplary pharmaceutically acceptable carriers include, for instance saline, solvents, dispersion media, coatings, antibacterial and antifungal agents, isotonic and absorption delaying agents, and the like, compatible with pharmaceutical administration.

In some embodiments, a pharmaceutical composition for use in accordance with the present disclosure may include and/or may be administered in conjunction with, one or more supplementary active compounds; in certain embodiments, such supplementary active agents can include ginger, curcumin, probiotics (e.g, probiotic strains of one or more of the following genera: Lactobacillus, Bifidobacterium, Saccharomyces, Enterococcus. Streptococcus, Pediococcus, Leuconostoc, Bacillus, and/or Escherichia coli (see Fijan, Int J Environ Res Public Health. 2014 May; 11(5): 4745-4767, which is incorporated herein by reference in its entirety); prebiotics (nondigestible food ingredients that help support growth of probiotic bacteria, e.g., fructans such as fructooligosaccharides (FOS) and inulins, galactans such as galactooligosaccharides (GOS), dietary fibers such as resistant starch, pectin, beta-glucans, and xylooligosaccharides (Hutkins et al., Curr Opin Biotechnol. 2016 Feb.; 37: 1-7, which is incorporated herein by reference in its entirety) and combinations thereof.

In some embodiments, a prebiotic comprises a fructooligosaccharide, an inulin, an isomaltooligosaccharide, a lactilol, a lactosucrose, a lactulose, a soy oligosaccharide, a transgalactooligosaccharide, a xylooligosaccharide, seaweed, or a combination thereof. In some embodiments, a prebiotic comprises seaweed. In some embodiments, a prebiotic comprises a pome extract, berry extract and walnut extract.

In some embodiments, a probiotic composition can be formulated for oral administration. In some embodiments, a probiotic composition can be a food, a beverage, a feed composition, or a nutritional supplement. In some embodiments, an ellagitannin composition, an enzymatic composition, or both can be a liquid, syrup, tablet, troche, gummy, capsule, powder, gel, or film. In some embodiments, a probiotic composition is an enteric-coated formulation.

In some embodiments, a probiotic comprises a prebiotic. In some embodiments, a prebiotic comprises a fructooligosaccharide, an inulin, an isomaltooligosaccharide, a lactilol, a lactosucrose, a lactulose, a soy oligosaccharide, a transgalactooligosaccharide, a xylooligosaccharide, seaweed, a pome extract, berry extract and walnut extract, or a combination thereof.

Pharmaceutical compositions are typically formulated to be compatible with its intended route of administration. Examples of routes of administration include oral administration, ophthalmic administration, intravitreal administration, or suprachoroidal administration. Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY), which is incorporated in its entirety by reference herein. Oral compositions generally include an inert diluent or an edible carrier (e.g. pharmaceutically acceptable diluent, pharmaceutically acceptable carrier). To give but a few examples, in some embodiments, an oral formulation may be or comprise a syrup, a liquid, a tablet, a troche, a gummy, a capsule, e.g., gelatin capsules, a powder, a gel, a film, etc. Similarly, ocular compositions (e.g. for ophthalmic, intravitreal, or suprachoroidal administration) may include an inert diluent or carrier (e.g. pharmaceutically acceptable diluent, pharmaceutically acceptable carrier), various additives such as viscosity enhancers, permeations enhancers, cyclodextrins, etc. Examples of viscosity enhancers include hydroxy methyl cellulose, hydroxy ethyl cellulose, sodium carboxy methyl cellulose, hydroxypropyl methyl cellulose and polyalcohol. Example of permeation enhancers include chelating agents, preservatives, surface active agents, bile salts, Benzalkonium chloride, polyoxyethylene glycol ethers (lauryl, stearyl and oleyl), ethylenediaminetetra acetic acid sodium salt, sodium taurocholate, saponins and cremophor EL, etc. For example, in some embodiments ocular formulations may be or comprise suspensions, emulsions (e.g. water-in-oil or oil-in water), nanocarriers, (e.g. nanoparticles, nanosuspensions, liposomes, nanomicelles, dendrimers, etc.) ointments, gels, eye drops, etc.

In some embodiments, pharmaceutically compatible binding agents, and/or adjuvant materials can be included as part of a pharmaceutical composition. In some particular embodiments, a pharmaceutical composition can contain, e.g., any one or more of the following inactive ingredients, or compounds of a similar nature: a binder such as microcrystalline cellulose, gum tragacanth or gelatin; an excipient such as starch or lactose, a disintegrating agent such as alginic acid, Primogel, or corn starch: a lubricant such as magnesium stearate or Sterotes: a glidant such as colloidal silicon dioxide; a sweetening agent such as sucrose or saccharin; or a flavoring agent such as peppermint, methyl salicylate, or orange flavoring. In some embodiments, the compositions can be taken as-is or sprinkled onto or mixed into a food or liquid (such as water). In some embodiments, a composition that may be administered to mammals as described herein may be or comprise an ingestible item (e.g., a food or drink) that comprises (e.g., is supplemented) with an individual microbial strain or combinations of microbial strains from a mammalian microbiome, extracts thereof, and/or components thereof.

In some embodiments, a food can be or comprise one or more of bars, candies, baked goods, cereals, salty snacks, pastas, chocolates, and other solid foods, as well as liquid or semi-solid foods including yogurt, soups and stews, and beverages such as smoothies, shakes, juices, and other carbonated or non-carbonated beverages. In some embodiments, foods are prepared by a subject by mixing in individual microbial strains or combinations of microbial strains from a mammalian microbiome, extracts thereof, and/or components thereof.

Compositions can be included in a kit, container, pack, or dispenser, together with instructions for administration or for use in a method described herein.

Those skilled in the art, reading the present disclosure, will appreciate that, in some embodiments, a composition (e.g., a pharmaceutical composition) as described herein may be or comprise one or more cells, tissues, or organisms (e.g., plant or microbe cells, tissues, or organisms) that produce (e.g., have produced, and/or are producing) a relevant compound.

Those skilled in the art will appreciate that, in some embodiments, technologies for preparing compositions and/or preparations, and/or for preparing (and particularly for preparing pharmaceutical compositions) may include one or more steps of assessing or characterizing a compound, preparation, or composition, e.g., as part of quality control. In some embodiments, if an assayed material does not meet pre-determined specifications for the relevant assessment, it is discarded. In some embodiments, if such assayed material does meet the pre-determined specifications, then it continues to be processed as described herein.

In some embodiments, a pharmaceutical composition provided herein can promote the colonization of an individual microbial strain or combinations of microbial strains from a mammalian microbiome, particularly microbial strain(s) that have been identified, characterized, or assessed as decreasing the severity or incidence of a mammalian disease, disorder, or condition, in a mammal suffering from or at risk of the mammalian disease, disorder, or condition. In some embodiments, a pharmaceutical composition provided herein can attenuate the colonization of an individual microbial strain or combinations of microbial strains from a mammalian microbiome, particularly microbial strain(s) that have been identified, characterized, or assessed as increasing the severity or incidence of a mammalian disease, disorder, or condition, in a mammal suffering from or at risk of the mammalian disease, disorder, or condition (e.g. eye disease, disorder, or condition). In some embodiments, a pharmaceutical composition provided herein can promote the colonization of an individual microbial strain or combinations of microbial strains from a mammalian microbiome, particularly microbial strain(s) that have been identified, characterized, or assessed as not affecting the severity or incidence of the mammalian disease, disorder, or condition but have been identified, characterized, or assessed as being capable of outcompeting one or more microbial strains that have been identified, characterized, or assessed as increasing the severity or incidence of a mammalian disease, disorder or condition, in a mammal suffering from or at risk of the mammalian disease, disorder, or condition.

In some embodiments, each of the one or more microbial strains in a composition comprises 10¹ colony forming units (CFUs) to 10²⁰ CFU. In some embodiments, each of the one or more microbial strains in a composition comprises 10¹ colony forming units (CFUs) to 10¹¹ CFU. In some embodiments, each of the one or more microbial strains in a composition comprises 10⁶ CFU to 10¹⁵ CFUs. In some embodiments, each of the one or more microbial strains in a composition comprises about 10¹ CFU to 10¹⁵ CFU, or about 10² CFU to 10¹⁴ CFU, or about 10³ CFU to 10¹³ CFU, or about 10⁴ CFU to 10¹³ CFU, or about 10⁵ CFU to 10¹² CFU, or about 10⁶ CFU to 10¹¹ CFU, or about 10⁷ CFU to 10¹⁰ CFU, or about 10⁸ CFU to 10⁹ CFU, or about 10⁵ CFU to 10¹⁰ CFU, or about 10⁸ CFU to 10¹² CFU. In some embodiments, each of the one or more microbial strains in a composition comprises at least about 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², or more CFUs. In some embodiments, each of the one or more microbial strains in a composition comprises at most about 10¹⁵, 5×10¹⁴, 10¹⁴, 5×10¹³, 10¹³, 5×10¹², 10¹², 5×10¹¹, 10¹¹, 5×10¹⁰, 10¹⁰, 5×10⁹, 10⁹, 5×10⁸, 10⁸, or less CFUs. In some embodiments, each of the one or more microbial strains in a composition comprises the same number of CFUs. In some embodiments, some of the one or more microbial strains in a composition comprises a different number of CFUs.

In some embodiments, a composition comprises a total of 10¹ CFU to 10²⁰ CFUs. In some embodiments, a composition comprises a total of 10⁶ CFU to 10¹⁵ of CFUs. In some embodiments, a composition can include about 10¹ CFU to 10²⁰ CFU, or about 10⁵ CFU to 10¹⁵ CFU, or about 10⁵ CFU to 10¹² CFU, about 10⁵ CFU to 10¹⁰ CFU, or about 10⁸ CFU to 10¹² CFU of one or more microbial strains. In some embodiments, a composition can include about 10¹ CFU to 10¹⁵ CFU, or about 10² CFU to 10¹⁴ CFU, or about 10³ CFU to 10¹³ CFU, or about 10⁴ CFU to 10¹³ CFU, or about 10⁵ CFU to 10¹² CFU, or about 10⁶ CFU to 10¹¹ CFU, or about 10⁷ CFU to 10¹⁰ CFU, or about 10⁸ CFU to 10⁹ CFU, or about 10⁵ CFU to 10¹⁰ CFU, or about 10⁸ CFU to 10¹² CFU of one or more microbial strains. In some embodiments, a composition can include at least 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², or more CFUs of one or more microbial strains. In some embodiments, a composition can include at most 10¹⁵, 5×10¹⁴, 10¹⁴, 5×10¹³, 10¹³, 5×10¹², 10¹², 5×10¹¹, 10¹¹, 5×10¹⁰, 10¹⁰, 5×10⁹, 10⁹, 5×10⁸, 10⁸, or less CFUs of one or more microbial strains.

In some embodiments, a pharmaceutical composition is tailored to a specific mammal (e.g., a specific human. e.g., a patient) based on that mammal's (e.g., human's) microbiome. In some embodiments, a pharmaceutical composition is specific for a microbiome of an individual mammal (e.g., human). In some embodiments, a pharmaceutical composition is specific for microbiomes of a population of mammals (e.g., humans). Populations of mammals can include, but are not limited to: families, mammals in the same regional location (e.g., neighborhood, city, state, or country), mammals with the same disease or condition, mammals of a particular age or age range, mammals that consume a particular diet (e.g., food, food source, or caloric intake).

Methods of Treatment

The present disclosure recognizes that compositions described herein can be useful in the treatment of subjects. Methods provided by the present disclosure include methods for the treatment of certain diseases, disorders and conditions. In some embodiments, relevant diseases, disorders and conditions may be or include an ocular disease, disorder, or condition. In some embodiments, an ocular disease, disorder, or condition may be AMD. In some embodiments, relevant diseases, disorders and conditions may be or include an ocular neovascular disease, disorder, or condition. In some embodiments, an ocular disease, disorder, or condition (e.g. ocular neovascular disease, disorder, or condition) may be macular degeneration related conditions, diabetic retinopathy, retinopathy of prematurity, retnitis pigmentosa, retinitis, glaucoma, proliferative vitreoretinopathy, uveitis, keratitis, and scleritis.

Generally, methods of treatment provided by the present disclosure involve administering a therapeutically effective amount of a composition as described herein alone or in combination with other compositions and/or treatments to a subject who is in need of, or who has been determined to be in need of, such treatment.

In some embodiments, methods of treatment provided herein are prophylactic or preventative, e.g., may be administered to subjects prior to display of significant symptoms and/or to exposure to a particular expected inducement that is associated with ocular diseases, disorders, or conditions described herein. In some embodiments, methods of treatment provided herein are therapeutic, e.g., may be administered to subjects after development of significant symptoms associated with ocular diseases, disorders, or conditions.

In some embodiments, provided methods of treatment are administered to a subject that is a mammal, e.g., a mammal that experiences a disease, disorder, or condition as described herein: in some embodiments, a subject is a human or non-human veterinary subject, e.g., an ape, cat dog, monkey, or pig.

In many embodiments, treatment involves ameliorating at least one symptom of a disease, disorder, or condition associated with ocular diseases, disorders, or conditions. In some embodiments, a method of treatment can be prophylactic.

In some embodiments, the methods can include administration of a therapeutically effective amount of compositions disclosed herein before, during (e.g., concurrently with), or after administration of a treatment that is expected to be associated with ocular diseases, disorders, or conditions.

In some embodiments, subjects who receive treatment as described herein may be receiving and/or may have received other treatment (e.g., pharmacological treatment/therapy, surgical, etc.), for example that may be intended to treat one or more symptoms or features of a disease disorder or condition as described herein (e.g. ocular diseases, disorders, or conditions), so that provided compositions are administered in combination with such other therapy (i.e. treatment) to treat the relevant disease, disorder, or condition.

In some embodiments, the compositions described herein can be administered in a form containing one or more pharmaceutically acceptable carriers. Suitable carriers have been described previously and vary with the desired form and mode of administration of a composition. For example, pharmaceutically acceptable carriers can include diluents or excipients such as fillers, binders, wetting agents, disintegrators, surface-active agents, glidants, and lubricants. Typically, a carrier may be a solid (including powder), liquid, or any combination thereof. Each carrier is preferably “acceptable” in the sense of being compatible with other ingredients in the composition and not injurious to a subject. A carrier can be biologically acceptable and inert (e.g., it permits the composition to maintain viability of the biological material until delivered to the appropriate site).

Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, orange flavoring, or other suitable flavorings. These are for purposes of example only and are not intended to be limiting.

Oral compositions can include an inert diluent or an edible carrier. For purposes of oral therapeutic administration, an active compound can be incorporated with excipients and used in the form of tablets, lozenges, pastilles, troches, or capsules, e.g., gelatin capsules. Oral compositions can also be prepared by combining a composition of the present disclosure with a food. In some embodiments, microbes (e.g. one or more microbial strains) can be formulated in a food item. Some non-limiting examples of food items to be used with the methods and compositions described herein include: popsicles, cheeses, creams, chocolates, milk, meat, drinks, pickled vegetables, kefir, miso, sauerkraut, etc. In other embodiments, food items can be juices, refreshing beverages, tea beverages, drink preparations, jelly beverages, and functional beverages; alcoholic beverages such as beers; carbohydrate-containing foods such as rice food products, noodles, breads, and pastas; paste products such as fish, hams, sausages, paste products of seafood; retort pouch products such as curries, food dressed with a thick starchy sauce, and Chinese soups; soups; dairy products such as milk, dairy beverages, ice creams, and yogurts; fermented products such as fermented soybean pastes, fermented beverages, and pickles; bean products; various confectionery products including biscuits, cookies, and the like, candies, chewing gums, gummies, cold desserts including jellies, cream caramels, and frozen desserts: instant foods such as instant soups and instant soy-bean soups; and the like. It is preferred that food preparations not require cooking after admixture with microbial strain(s) to avoid killing any microbes. In one embodiment a food used for administration is chilled, for example, iced flavored water. In certain embodiments, the food item is not a potentially allergenic food item (e.g., not soy, wheat, peanut, tree nuts, dairy, eggs, shellfish or fish). Pharmaceutically compatible binding agents, and/or adjuvant materials can be included as part of the composition.

Ocular formulations (e.g. for ophthalmic, intravitreal, or suprachoroidal administration) can include an inert diluent or a carrier. For purposes of ocular therapeutic administration, an active compound can be incorporated with excipients and used in the form of suspensions, emulsions (e.g. water-in-oil or oil-in water), nanocarriers. (e.g. nanoparticles, nanosuspensions, liposomes, nanomicelles, dendrimers, etc.) ointments, gels, eye drops, etc. In some embodiments, administration of such formulations is topical (e.g. eye drops). In some embodiments, administration of such formulations is via injection (e.g. intravitreal, suprachoroidal, etc.).

In some such embodiments, a composition described herein is administered to a subject according to a dosing regimen that achieves population of the subject's microbiome with administered cells. In some embodiments, a composition is administered to a subject in a single dose. In some embodiments, a composition is administered to a subject in a plurality of doses. In some embodiments, a dose of a composition is administered to a subject twice a day, daily, weekly, or monthly.

In some embodiments, each of the one or more microbial strains in a dose comprises 10¹ to 10⁵ colony forming units (CFUs). In some embodiments, each of the one or more microbial strains in a dose comprises 10⁶ to 10¹⁵ CFUs. In some embodiments, each of the one or more microbial strains in a dose comprises the same number of CFUs. In some embodiments, some of the one or more microbial strains in a dose comprises a different number of CFUs.

In some embodiments, a dose of one or more microbial strains comprises a total of 10⁶ to 10¹⁵ CFUs. In some embodiments, a dose of one or more microbial strains comprises a total of 10⁷ to 10¹⁵ CFUs. In some embodiments, a dose of one or more microbial strains comprises 5-200 billion CFUs. In some embodiments, a dose of one or more microbial strains comprises 5-50 billion CFUs. In some embodiments, a dose of one or more microbial strains comprises 5-20 billion CFUs. In some embodiments, a dose of one or more microbial strains comprises 50-100 billion CFUs. In some embodiments, a dose of one or more microbial strains comprises 100-200 billion CFUs.

In some embodiments, efficacy can be assessed by measuring the degree of oxidative stress of cells in a biological sample prior to and following administration of a composition as described herein. The degree of oxidative stress of cells can be assessed by, for example, measuring the expression of oxidative stress biomarkers, such as reactive oxygen species (ROS) levels, or lipid, protein, and nucleic acid damage levels, or by determining the ratio of oxidized to reduced forms of one or more biomarkers. High levels of oxidative stress can be cytotoxic, so the degree of oxidative stress can be measured by assessing the concentration of intracellular proteins present in the systemic circulation from inflamed or lysed cells (e.g. ocular cells).

### EXEMPLIFICATION

### Example 1: Evaluation of Cytotoxicity of Sodium Iodate (NaIO₃) Using MTT Assay

Purpose: This Example evaluates the cytotoxicity of sodium idoate (NaIO₃) and characterizes Human retinal pigment epithelial cells (ARPE-19) degradation as an in vitro model for AMD.

Cell Culture: Human retinal pigment epithelial cells (ARPE-19) passages 3-7 were used for all experiments. Cells were cultured in 96-well plates in DMEM:F12 with 10% of FBS, and incubated at 37° C. with 5% CO₂ humidified atmosphere. The medium was renewed every 2 days.

Cell Viability Assay: The colorimetric 3-(4,5-dimethylthiazol-2-yl)-2,5-diphenyl tetrazolium bromide (MTT) assay was used to check cell viability. ARPE-19 cells were cultured into 96 wells plate and divided into the control group and sodium iodate (NaIO₃) group (n≥3 per group). In the control group, cells were treated only with DMEM:F12. Different doses of NaIO₃ (6-1200 μg/ml) was given to the NaIO₃ group. After 24 hours of incubation, the absorbance cell viability was evaluated by spectrophotometrically using a microplate reader (Promega, Explorer™) at 600 nm.

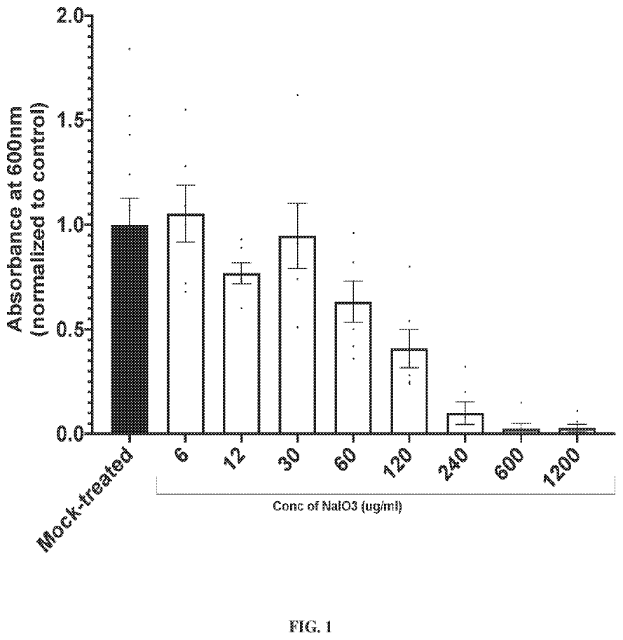

Results: The results showed that the increasing concentrations of NaIO₃ (6, 12, 30, 60, 120, 240, 600, 1200 μg/ml) resulted in increased toxicity in ARPE-19 cells (FIG. 1). As shown in FIG. 1, lower concentrations of NaIO₃ (e.g. 6, 12, 30 μg/ml) resulted in minimal loss to cell viability, and higher concentrations of NaIO₃ (e.g. 600 μg/ml and 1200 μg/ml) resulted in complete loss of cell viability.

### Example 2: Effect of MBTs Comprising One Microbial Strain on NaIO₃-Induced Retinal Degeneration

Purpose: This Example evaluates the effect of various microbiome therapies (MBTs), each MBT comprising one microbial strain, on NaIO₃-induced degradation of ARPE-19 cells.

Cell Culture: ARPE-19 passages 3-7 were used for all experiments. Cells were cultured in %-well plates in DMEM:F12 with 10% of FBS, and incubated at 37° C. with 5% CO₂ humidified atmosphere. The medium was renewed every 2 days.

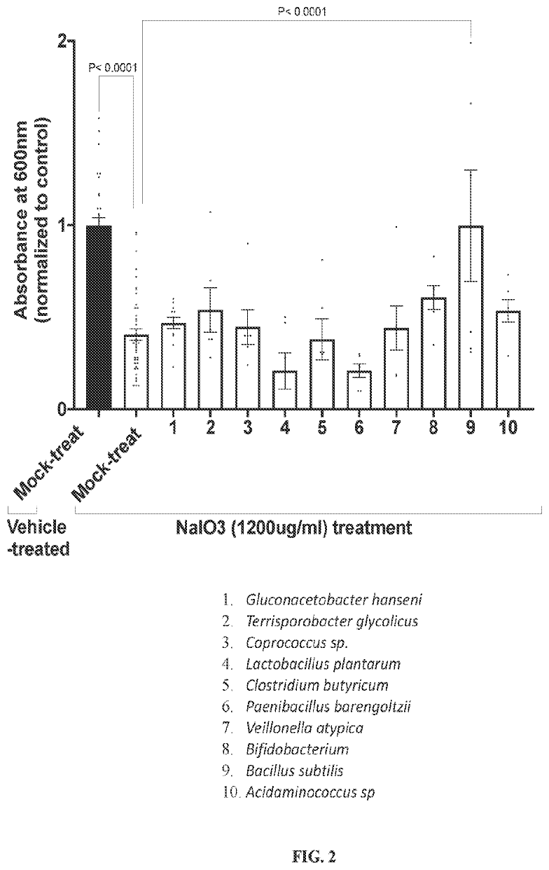

Cell Viability Assay: The colorimetric MTT assay was used to check cell viability. ARPE-19 cells were cultured into 96 wells plate and divided into the control group, sodium iodate (NaIO₃) group (1200 μg/ml of NaIO₃ only), and treatment group (1200 μg/ml of NaIO₃÷MBT)(n≥3 per group). In the control group, cells were treated only with DMEM:F12 (control media; labeled ‘mock-treat vehicle treated’ in FIG. 2). The NaIO₃ group (labeled ‘mock-treat’ in NaIO₃ treatment group in FIG. 2) and treatment group (labeled 1 through 10 in FIG. 2), were treated with 1200 μg/ml of NaIO₃. At the same time different MBTs, labeled 1 through 10 and summarized in FIG. 2 and Table 2 below, were given to the treatment group at a concentration of 10⁶ CFU. After 16 hours of incubation, the absorbance cell viability was evaluated by spectrophotometrically using a microplate reader at 600 nm.

TABLE 2
MBTs evaluated

| # | MBT |
|---|---|
| 1 | Gluconacetobacter hanseni |
| 2 | Terrisporobacter glycolicus |
| 3 | Coprococcus sp. |
| 4 | Lactobacillus plantarum |
| 5 | Clostridium butyricum |
| 6 | Paenibacillus barengoltzii |
| 7 | Veillonella atypica |
| 8 | Bifidobacterium |
| 9 | Bacillus subtilis |
| 10 | Acidaminococcus sp |

Results: Results showed that treatment of NaIO₃-treated ARPE-19 cells with any of MBTs 1 through 10 resulted in reduced toxicity of the ARPE-19 cells compared to controls (FIG. 2). As shown in FIG. 2, treatment with MBTs 1-10 reduced the cytotoxic effects of 1200 μg/ml NaIO₃ and resulted in improved cell viability. Specifically, treatment with MBT #9 (Bacillus subtilis) resulted in almost complete inhibition of loss of cell viability due to NaIO₃. Thus. MBT #9 is able to suppress NaIO₃ induced ARPE-19 cell death.

### Example 3: Effect of MBTs Comprising Multiple Microbial Strains on NaIO₃Induced Retinal Degeneration

Purpose: This Example evaluates the effect of microbiome therapies (MBTs) comprising multiple microbial strains on NaIO₃-induced degradation of ARPE-19 cells.

Cell Culture: ARPE-19 passages 3-7 were used for all experiments. Cells were cultured in 96-well plates in DMEM:F12 with 10% of FBS, and incubated at 37° C. with 5% CO₂ humidified atmosphere. The medium was renewed every 2 days.

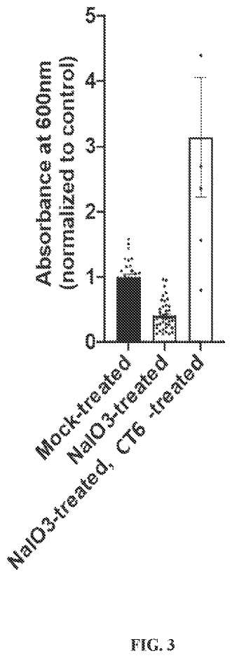

Cell Viability Assay: The colorimetric MTT assay was used to check cell viability. ARPE-19 cells were cultured into 96 wells plate and divided into the control group, sodium iodate (NaIO₃) group (1200 μg/ml of NaIO₃ only), and treatment group (1200 μg/ml of NaIO₃÷MBT)(n≥3 per group). In the control group, cells were treated only with DMEM:F12 (control media; labeled ‘mock-treated’ in FIG. 3). The NaIO₃ group (labeled ‘NaIO₃-treated’ in FIG. 3) and treatment group (labeled ‘NaIO₃-treated, CT6 treated’ in FIG. 3), were treated with 1200 μg/ml of NaIO₃. At the same time an MBT composition (also named CT6) was given to the treatment group at a concentration of 6×10⁶ CFU. CT6 is a combination of six microbial strains, namely Gluconacetobacter hanseni. Terrisporobacter glycolicus, Coprococcus sp., Lactobacillus plantarum, Veillonella atypica, and Bifidobacterium, each at a concentration of 10⁶ CFU. After 16 hours of incubation, the absorbance cell viability was evaluated by spectrophotometrically using a microplate reader at 600 nm.

Results: Results showed that treatment of NaIO₃-treated ARPE-19 cells with CT6 resulted in reduced toxicity of the ARPE-19 cells compared to controls (FIG. 3). As shown in FIG. 3, treatment with CT6 reduced the cytotoxic effects of 1200 μg/ml NaIO₃ and resulted in improved and increased (2-3× increase) cell viability. That is, this Example demonstrates that CT6 was not only able to suppress the NaIO₃-induced ARPE-19 cell death, but also increase the cell viability by 2-3-fold.

### Example 4: Effect of Bacterial Metabolite 2-Keto-Gluconate on NaIO₃-Induced Retinal Degeneration

Purpose: This Example evaluates the effect of a bacterial metabolite, 2-keto-gluconate, on NaIO₃-induced degradation of ARPE-19 cells.

Cell Culture: ARPE-19 passages 3-7 were used for all experiments. Cells were cultured in 96-well plates in DMEM:F12 with 10% of FBS, and incubated at 37° C. with 5% CO₂ humidified atmosphere. The medium was renewed every 2 days.

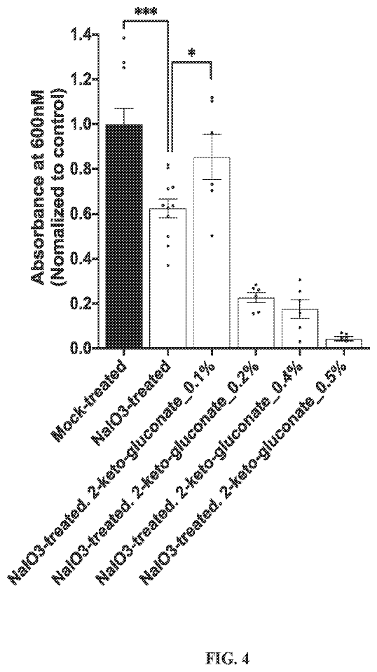

Cell Viability Assay: The colorimetric MTT assay was used to check cell viability. ARPE-19 cells were cultured into 96 wells plate and divided into the control group, sodium iodate (NaIO₃) group (1200 μg/ml of NaIO₃ only), and treatment group (1200 μg/ml of NaIO₃÷2-keto-gluconate)(n≥3 per group). In the control group, cells were treated only with DMEM:F12 (control media; labeled ‘mock-treated’ in FIG. 4). The NaIO₃ group (labeled ‘NaIO₃-treated’ in FIG. 4) and treatment group (labeled ‘NaIO₃-treated, 2-keto-gluconate’ in FIG. 4), were treated with 1200 μg/ml of NaIO₃. At the same time different doses of 2-keto-gluconate (0.1%, 0.2%, 0.4%, and 0.5% w/v) was given to the treatment group. After 16 hours of incubation, the absorbance cell viability was evaluated by spectrophotometrically using a microplate reader at 600 nm.

Results: Results showed that treatment of NaIO₃-treated ARPE-19 cells with 2-keto-gluconate resulted in reduced toxicity of the ARPE-19 cells as compared to controls (FIG. 4). As shown in FIG. 4, treatment with 0.1% of 2-keto-gluconate reduced the cytotoxic effects of 1200 μg/ml NaIO₃ and resulted in improved cell viability. Thus, this Example demonstrates that 2-keto-gluconate is able to suppress NaIO₃-induced ARPE-19 cell death.

### Example 5: Effect of Bacterial Metabolite 5-Keto-Gluconate on NaIO₃-Induced Retinal Degeneration

Purpose: This Example evaluates the effect of a bacterial metabolite, 5-keto-gluconate, on NaIO₃-induced degradation of ARPE-19 cells.

Cell Culture ARPE-19 passages 3-7 were used for all experiments. Cells were cultured in 96-well plates in DMEM:F12 with 10% of FBS, and incubated at 37° C. with 5% CO₂ humidified atmosphere. The medium was renewed every 2 days.

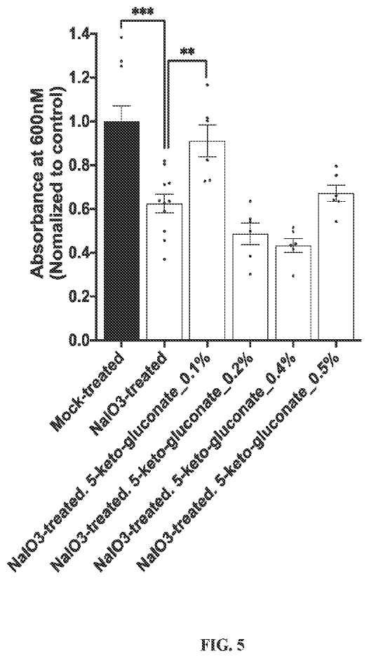

Cell Viability Assay: The colorimetric MTT assay was used to check cell viability. ARPE-19 cells were cultured into 96 wells plate and divided into the control group, sodium iodate (NaIO₃) group (1200 μg/ml of NaIO₃ only), and treatment group (1200 μg/ml of NaIO₃÷5-keto-gluconate)(n≥3 per group). In the control group, cells were treated only with DMEM:F12 (control media; labeled ‘mock-treated’ in FIG. 5). The NaIO₃ group (labeled ‘NaIO₃-treated’ in FIG. 5) and treatment group (labeled ‘NaIO₃-treated, 5-keto-gluconate’ in FIG. 5), were treated with 1200 μg/ml of NaIO₃. At the same time different doses of 5-keto-gluconate (0.1%, 0.2%, 0.4%, and 0.5% w/v) was given to the treatment group. After 16 hours of incubation, the absorbance cell viability was evaluated by spectrophotometrically using a microplate reader at 600 nm.

Results: Results showed that treatment of NaIO₃-treated ARPE-19 cells with 5-keto-gluconate resulted in reduced toxicity of the ARPE-19 cells as compared to controls (FIG. 5). As shown in FIG. 5, treatment with all tested concentrations of 5-keto-gluconate reduced the cytotoxic effects of 1200 μg/ml NaIO₃ and resulted in improved cell viability. Thus, this Example demonstrates that 5-keto-gluconate is able to suppress NaIO₃-induced ARPE-19 cell death.

### OTHER EMBODIMENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice are hereby incorporated by reference in their entireties.

It is to be understood that while embodiments of the invention have been described in conjunction with the detailed description thereof, the foregoing description is intended to illustrate and not limit the scope of the invention, which is defined by the scope of the appended claims. Other aspects, advantages, and modifications are within the scope of the following claims.

APPENDIX 1-1
Metabolite Abbreviations

| Candidates^{†} | Pathway Label^{‡} | Pathway Index^{§} |
|---|---|---|
| 1,3-Diaminopropane | DAP | Urea cycle relating metaboloism |
| 1-Methyl-4-imidazoleacetic acid | MIA | Urea cycle relating metaboloism |
| 1-Methylhistamine | 1-Methylhistamine | Urea cycle relating metaboloism |
| 1-Methylnicotinamide | 1-Methylnicotinamide | Metabolism of coenzymes |
| 1-Pyrroline 5-carboxylic acid | P5C | Urea cycle relating metaboloism |
| 2,3-Diphosphoglyceric acid | Diphosphoglycerate | Central carbon metabolism |
| 2,5-Dihydroxybenzoic acid | Gensigen | Pathway overview |
| 2-Aminoadipic acid | 2-Aminoadipic acid | Lipid and amino acid metabolism |
| 2′-Deoxyadenosine | dAdenosine | Nucleotide metabolism |
| 2′-Deoxycytidine | dCyt | Nucleotide metabolism |
| 2′-Deoxyguanosine | dGuanosine | Nucleotide metabolism |
| 2′-Deoxyuridine | dUri | Nucleotide metabolism |
| 2-Hydroxybutyric acid | 2-HBA | Lipid and amino acid metabolism |
| 2-Oxoadipic acid | 2-Oxoadipic acid | Lipid and amino acid metabolism |
| 2-Oxobutyric acid | 2-Oxobutyric acid | Lipid and amino acid metabolism |
| 2-Oxoglutaric acid | 2-OG | Central carbon metabolism/ |
| | | Urea cycle relating metaboloism |
| 2-Oxoisovaleric acid | 2-KIV | BCAA & aromatic amino acids |
| 2-Phenylethylamine | Phenylethylamine | BCAA & aromatic amino acids |
| 2-Phosphoglyceric acid | 2-PG | Central carbon metabolism |
| 3,3′,5-Triiodothyronine | T3 | BCAA & aromatic amino acids |
| 3,4-Dihydroxyphenylglycol | DHPG | Pathway overview |
| 3,5-Diiodotyrosine | 3,5-DI-Tyr | BCAA & aromatic amino acids |
| 3-Aminoisobutyric acid | 3-Aminoisobutyric acid | BCAA & aromatic amino acids/ |
| | | Nucleotide metabolism |
| 3′-Dephospho CoA | Dephospho CoA | Metabolism of coenzymes |
| 3-Hydroxyanthranilic acid | 3-OHAA | BCAA & aromatic amino acids |
| 3-Hydroxybutyric acid | 3-HBA | Central carbon metabolism/ |
| | | Lipid and amino acid metabolism |
| 3-Hydroxykynurenine | 3-OHKY | BCAA & aromatic amino acids |
| 3-Hydroxypropionic acid | b-Lactate | BCAA & aromatic amino acids |
| 3-Iodotyrosine | MIT | BCAA & aromatic amino acids |
| 3-Methoxy-4-hydroxyphenylethyleneglycol | MHPG | BCAA & aromatic amino acids |
| 3-Methoxyanthranilic acid | 3-Methoxyanthranilic acid | BCAA & aromatic amino acids |
| 3-Methoxytyramine | 3-Methoxytyramine | BCAA & aromatic amino acids |
| 3-Methyl-2-oxovaleric acid | 2K3MVA | BCAA & aromatic amino acids |
| 3-Methylcrotonyl CoA_divalent | 3-Methylcrotonyl CoA | BCAA & aromatic amino acids |
| 3-Methylhistidine | 3-Methylhistidine | Urea cycle relating metaboloism |
| 3-Phosphoglyceric acid | 3-PG | Central carbon metabolism/ |
| | | Lipid and amino acid metabolism |
| 3-Ureidopropionic acid | 3-Ureidopropionic acid | Nucleotide metabolism |
| 4-Acetamidobutanoic acid | 4-Acetamidobutanoic acid | Pathway overview |
| 4-Guanidinobutyric acid | 4-GBA | Urea cycle relating metaboloism |
| 4-Hydroxyphenylacetaldehyde | 4-Hydroxyphenylacetaldehyde | Pathway overview |
| 4-Methyl-2-oxovaleric acid | 2-Oxoleucine | BCAA & aromatic amino acids |
| 4-Methylthio-2-oxobutyric acid | KMTB | Lipid and amino acid metabolism |
| 4-Pyridoxic acid | 4-Pyridoxic acid | Metabolism of coenzymes |
| 5,6-Dimethylbenzimidazole | Dimethylbenzimidazole | Metabolism of coenzymes |
| 5-Amino-4-oxovaleric acid | 5-ALA | Lipid and amino acid metabolism |
| 5-Aminoimidazole-4-carboxamide ribotide | AICAR | Nucleotide metabolism |
| 5′-Deoxy-5′-methylthioadenosine | MTA | Urea cycle relating metaboloism |
| 5-Hydroxyindoleacetic acid | 5-Hydroxy-IAA | BCAA & aromatic amino acids |
| 5-Hydroxylysine | 5-Hydroxylysine | Lipid and amino acid metabolism |
| 5-Hydroxytryptophan | Pretonine | BCAA & aromatic amino acids |
| 5-Methoxyindoleacetic acid | 5-MIAA | BCAA & aromatic amino acids |
| 5-Methoxytryptamine | 5MOT | BCAA & aromatic amino acids |
| 5-Methyltetrahydrofolic acid | 5-MTHF | Metabolism of coenzymes |
| 5-Oxoproline | Oxoproline | Urea cycle relating metaboloism |
| 6-Phosphogluconic acid | 6-PG | Central carbon metabolism |
| 7,8-Dihydrofolic acid | Dihydrofolic acid | Metabolism of coenzymes |
| Acetanilide | Acetanilide | BCAA & aromatic amino acids |
| Acetoacetic acid | Acetoacetic acid | Central carbon metabolism/ |
| | | Lipid and amino acid metabolism |
| Acetoacetyl CoA_divalent | AAcCoA | Lipid and amino acid metabolism |
| Acetyl CoA_divalent | AcCoA | Central carbon metabolism/ |
| | | Lipid and amino acid metabolism/ |
| | | Metabolism of coenzymes |
| Acetylcholine | Acetylcholine | Lipid and amino acid metabolism |
| Adenine | Adenine | Nucleotide metabolism |
| Adenosine | Adenosine | Nucleotide metabolism |
| Adenylosuccinic acid | Succinyl AMP | Nucleotide metabolism |
| ADP | ADP | Central carbon metabolism/ |
| | | Nucleotide metabolism |
| ADP-ribose | ADP-Rib | Central carbon metabolism/ |
| | | Metabolism of coenzymes |
| Adrenaline | Adrenaline | BCAA & aromatic amino acids |
| Agmatine | Agmatine | Urea cycle relating metaboloism |
| Ala | Ala | Central carbon metabolism/ |
| | | Urea cycle relating metaboloism/ |
| | | BCAA & aromatic amino acids |
| Allantoic acid | Allantoic acid | Pathway overview |
| AMP | AMP | Nucleotide metabolism |
| Anserine_divalent | Anserine | Urea cycle relating metaboloism |
| Anthranilic acid | Anthranilic acid | BCAA & aromatic amino acids |
| Arg | Arg | Central carbon metabolism/ |
| | | Urea cycle relating metaboloism |
| Argininosuccinic acid | ArgSuccinate | Urea cycle relating metaboloism |
| Ascorbate 2-glucoside | Ascorbate 2-glucoside | Metabolism of coenzymes |
| Ascorbate 2-phosphate | Ascorbate 2-phosphate | Metabolism of coenzymes |
| Ascorbate 2-sulfate | Ascorbate 2-sulfate | Metabolism of coenzymes |
| Ascorbic acid | Ascorbic acid | Metabolism of coenzymes |
| Asn | Asn | Urea cycle relating metaboloism |
| Asp | Asp | Central carbon metabolism/ |
| | | Urea cycle relating metaboloism/ |
| | | Nucleotide metabolism |
| ATP | ATP | Central carbon metabolism/ |
| | | Nucleotide metabolism |
| Betaine | Betaine | Lipid and amino acid metabolism |
| Betaine aldehyde_+ H₂O | BTL | Lipid and amino acid metabolism |
| Biotin | Biotin | Metabolism of coenzymes |
| cAMP | cAMP | Nucleotide metabolism |
| Carbamoylphosphate | Carbamoyl-P | Urea cycle relating metaboloism |
| Carnitine | Carnitine | Lipid and amino acid metabolism |
| Carnosine | Carnosine | Urea cycle relating metaboloism |
| CDP | CDP | Nucleotide metabolism |
| CDP-choline | CDP-choline | Lipid and amino acid metabolism |
| cGMP | cGMP | Nucleotide metabolism |
| Cholic acid | Cholic acid | Lipid and amino acid metabolism |
| Choline | Choline | Lipid and amino acid metabolism |
| cis -Aconitic acid | cis -Aconitic acid | Central carbon metabolism |
| cis-Hydroxyproline | cis-Hydroxyproline | Urea cycle relating metaboloism |
| Citramalic acid | Citramalic acid | Pathway overview |
| Citric acid | Citric acid | Central carbon metabolism |
| Citrulline | Citrulline | Urea cycle relating metaboloism |
| CMP | CMP | Nucleotide metabolism |
| CMP-N-acetylneuraminate | CMP-NeuNAc | Central carbon metabolism |
| CoA_divalent | CoA | Central carbon metabolism/ |
| | | Metabolism of coenzymes |
| Creatine | Creatine | Urea cycle relating metaboloism |
| Creatinine | Creatinine | Urea cycle relating metaboloism |
| CTP | CTP | Nucleotide metabolism |
| Cys | Cys | Urea cycle relating metaboloism/ |
| | | Lipid acid metabolism |
| Cys-Gly | Cys-Gly | Urea cycle relating metaboloism |
| Cystathionine | Cystathionine | Lipid and amino acid metabolism |
| Cysteamine | Cysteamine | Lipid and amino acid metabolism |
| Cysteic acid | Cysteic acid | Lipid and amino acid metabolism |
| Cysteinesulfinic acid | Cysteinesulfinic acid | Lipid and amino acid metabolism |
| Cystine | Cystine | Lipid and amino acid metabolism |
| Cytidine | Cytidine | Nucleotide metabolism |
| dADP | dADP | Nucleotide metabolism |
| dAMP | dAMP | Nucleotide metabolism |
| dATP | dATP | Nucleotide metabolism |
| dCDP | dCDP | Nucleotide metabolism |
| dCMP | dCMP | Nucleotide metabolism |
| dCTP | dCTP | Nucleotide metabolism |
| Deamido-NAD⁺ | Deamido-NAD | Metabolism of coenzymes |
| Desthiobiotin | Desthiobiotin | Metabolism of coenzymes |
| dGDP | dGDP | Nucleotide metabolism |
| dGMP | dGMP | Nucleotide metabolism |
| dGTP | dGTP | Nucleotide metabolism |
| Dihydroorotic acid | Dihydroorotic acid | Nucleotide metabolism |
| Dihydrouracil | Dihydrouracil | Nucleotide metabolism |
| Dihydroxyacetone phosphate | DHAP | Central carbon metabolism/ |
| | | Lipid and amino acid metabolism |
| dIMP | dIMP | Nucleotide metabolism |
| dITP | dITP | Nucleotide metabolism |
| DOPA | DOPA | BCAA & aromatic amino acids |
| Dopamine | Dopamine | BCAA & aromatic amino acids |
| dTDP | dTDP | Nucleotide metabolism |
| dTDP-glucose | TDP-Glc | Pathway overview |
| dTMP | dTMP | Nucleotide metabolism |
| dTTP | dTTP | Nucleotide metabolism |
| dUDP | dUDP | Nucleotide metabolism |
| dUMP | dUMP | Nucleotide metabolism |
| dUTP | dUTP | Nucleotide metabolism |
| Ergothioneine | Ergothioneine | Pathway overview |
| Erythrose 4-phosphate | E4P | Central carbon metabolism |
| FAD_divalent | FAD | Metabolism of coenzymes |
| FMN | FMN | Metabolism of coenzymes |
| Folic acid | Folic acid | Metabolism of coenzymes |
| Formylanthranilic acid | Formylanthranilate | Pathway overview |
| Fructose 1,6-diphosphate | F1,6P | Central carbon metabolism |
| Fructose 1-phosphate | D-F1P | Central carbon metabolism |
| Fructose 6-phosphate | F6P | Central carbon metabolism |
| Fumaric acid | Fumaric acid | Central carbon metabolism/ |
| | | Urea cycle relating metaboloism |
| GABA | GABA | Urea cycle relating metaboloism |
| Galactose 1-phosphate | Gal1P | Central carbon metabolism |
| GDP | GDP | Nucleotide metabolism |
| GDP-fucose | GDP-fucose | Central carbon metabolism |
| GDP-mannose | GDP-Man | Central carbon metabolism |
| Gln | Gln | Urea cycle relating metaboloism |
| Glu | Glu | Central carbon metabolism/ |
| | | Urea cycle relating metaboloism |
| Glucosamine | Glucosamine | Central carbon metabolism |
| Glucosamine 6-phosphate | Glc-6P | Central carbon metabolism |
| Glucosaminic acid | Glucosaminic acid | Central carbon metabolism |
| Glucose 1-phosphate | G1P | Central carbon metabolism |
| Glucose 6-phosphate | G6P | Central carbon metabolism |
| Glucuronic acid | Glucuronic acid | Central carbon metabolism |
| Glutaryl CoA_divalent | Glutaryl-CoA | Lipid and amino acid metabolism |
| Glutathione (GSH) | GSH | Urea cycle relating metaboloism |
| Glutathione (GSSG)_divalent | GSSG | Urea cycle relating metaboloism |
| Gly | Gly | Urea cycle relating metaboloism/ |
| | | Lipid and amino acid metabolism |
| Glyceraldehyde 3-phosphate | GAP | Central carbon metabolism/ |
| | | Lipid and amino acid metabolism |
| Glyceric acid | Glyceric acid | Central carbon metabolism/ |
| | | Lipid and amino acid metabolism |
| Glycerol 3-phosphate | G3P | Central carbon metabolism/ |
| | | Lipid and amino acid metabolism |
| Glycerophosphocholine | GPCho | Lipid and amino acid metabolism |
| Glycocholic acid | Glycocholic acid | Lipid and amino acid metabolism |
| Glycolic acid | Glycolic acid | Lipid and amino acid metabolism |
| Glyoxylic acid | Glyoxylic acid | Lipid and amino acid metabolism |
| GMP | GMP | Nucleotide metabolism |
| GTP | GTP | Nucleotide metabolism |
| Guanidoacetic acid | Guanidoacetic acid | Urea cycle relating metaboloism |
| Guanine | Guanine | Nucleotide metabolism |
| Guanosine | Guanosine | Nucleotide metabolism |
| His | His | Urea cycle relating metaboloism |
| Histamine | Histamine | Urea cycle relating metaboloism |
| HMG CoA_divalent | HMG-COA | Lipid and amino acid metabolism |
| Homocysteine | Homocysteine | Lipid and amino acid metabolism |
| Homovanillic acid | HVA | BCAA & aromatic amino acids |
| Hydroxyproline | Hydroxyproline | Urea cycle relating metaboloism |
| Hypotaurine | Hypotaurine | Lipid and amino acid metabolism |
| Hypoxanthine | Hypoxanthine | Nucleotide metabolism |
| IDP | IDP | Nucleotide metabolism |
| Ile | Ile | BCAA & aromatic amino acids |
| Imidazole-4-acetic acid | Imidazole-4-acetic acid | Urea cycle relating metaboloism |
| IMP | IMP | Nucleotide metabolism |
| Indole-3-acetaldehyde | Indoleacetaldehyde | BCAA & aromatic amino acids |
| Indole-3-acetic acid | Indole-3-acetic acid | BCAA & aromatic amino acids |
| Inosine | Inosine | Nucleotide metabolism |
| Isobutyryl CoA_divalent | Isobutyryl-CoA | Lipid and amino acid metabolism/ |
| | | BCAA & aromatic amino acids |
| Isocitric acid | Isocitric acid | Central carbon metabolism |
| ITP | ITP | Nucleotide metabolism |
| Kynurenic acid | Kynurenic acid | BCAA & aromatic amino acids |
| Kynurenine | Kynurenine | BCAA & aromatic amino acids |
| Lactic acid | Lactic acid | Central carbon metabolism/ |
| | | Urea cycle relating metaboloism |
| Leu | Leu | BCAA & aromatic amino acids |
| Lys | Lys | Lipid and amino acid metabolism |
| Malic acid | Malic acid | Central carbon metabolism/ |
| | | Urea cycle relating metaboloism |
| Malonyl CoA_divalent | Malonyl-CoA | Central carbon metabolism/ |
| | | Lipid and amino acid metabolism |
| Mannose 1-phosphate | Man1P | Central carbon metabolism |
| Mannose 6-phosphate | Man6P | Central carbon metabolism |
| Melatonin | Melatonin | BCAA & aromatic amino acids |
| Met | Met | Lipid and amino acid metabolism |
| Methylmalonic acid | Methylmalonic acid | Lipid and amino acid metabolism/ |
| | | BCAA & aromatic amino acid |
| N,N-Dimethylglycine | DMG | Lipid and amino acid metabolism |
| N⁶,N⁶,N⁶-Trimethyllysine | Trimethyllysine | Lipid and amino acid metabolism |
| N-Acetylaspartic acid | N-Acetylaspartic acid | Urea cycle relating metaboloism |
| N-Acetylglucosamine | GlcNAc | Central carbon metabolism |
| N-Acetylglucosamine 1-phosphate | GlcNAc-P | Central carbon metabolism |
| N-Acetylglucosamine 6-phosphate | NAcGlcNP | Central carbon metabolism |
| N-Acetylglutamic acid | N-AcGlu | Urea cycle relating metaboloism |
| N-Acetylmannosamine | ManNAc | Central carbon metabolism |
| N-Acetylneuraminic acid | NeuNAc | Central carbon metabolism |
| N-Acetylornithine | N-AcOrn | Urea cycle relating metaboloism |
| N-Acetylputrescine | N-Acetylputrescine | Urea cycle relating metaboloism |
| NAD⁺ | NAD⁺ | Central carbon metabolism/ |
| | | Metabolism of coenzymes |
| NADH | NADH | Central carbon metabolism/ |
| | | Metabolism of coenzymes |
| NADP⁺ | NADP⁺ | Central carbon metabolism/ |
| | | Metabolism of coenzymes |
| NADPH_divalent | NADPH | Central carbon metabolism/ |
| | | Metabolism of coenzymes |
| N-Carbamoylaspartic acid | Carbamoyl-Asp | Urea cycle relating metabolism/ |
| | | Nucleotide metabolism |
| N-Formylaspartic acid | N-Formyl aspartic acid | Urea cycle relating metaboloism |
| Nicotinamide | Nicotinamide | Metabolism of coenzymes |
| Nicotinic acid | Nicotinic acid | Metabolism of coenzymes |
| N-Methylserotonin | N-Methylserotonin | Pathway overview |
| N-Methyltryptamine | N-Methyltryptamine | BCAA & aromatic amino acids |
| N-Methyltyramine | N-Methyltyramine | BCAA & aromatic amino acids |
| NMN | NicRN | Metabolism of coenzymes |
| Noradrenaline | Noradrenaline | BCAA & aromatic amino acids |
| Normetanephrine | Normetanephrine | Pathway overview |
| O-Acetylcarnitine | ALCAR | Lipid and amino acid metabolism |
| o-Aminophenol | 2-Aminophenol | BCAA & aromatic amino acids |
| o-Hydroxyphenylacetic acid | 2-HPAA | BCAA & aromatic amino acids |
| O-Phosphoserine | 3PSer | Lipid and amino acid metabolism |
| Ornithine | Ornithine | Urea cycle relating metaboloism |
| Orotic acid | Orotic acid | Nucleotide metabolism |
| Orotidine 5′-monophosphate | Orotidine 5′P | Nucleotide metabolism |
| P¹,P⁴-Di(adenosine-5′) | AppppA | Nucleotide metabolism |
| tetraphosphate_divalent | | |
| Pantothenic acid | Pantothenic acid | Metabolism of coenzymes |
| Phe | Phe | BCAA & aromatic amino acids |
| Phenaceturic acid | Phenaceturic acid | BCAA & aromatic amino acids |
| Phenylpyruvic acid | Phenylpyruvate | BCAA & aromatic amino acids |
| Phosphocreatine | Phosphocreatine | Urea cycle relating metaboloism |
| Phosphoenolpyruvic acid | PEP | Central carbon metabolism |
| Phosphorylcholine | Phosphorylcholine | Lipid and amino acid metabolism |
| p-Hydroxyphenylacetic acid | 4-HPAA | BCAA & aromatic amino acids |
| p-Hydroxyphenylpyruvic acid | HPP | BCAA & aromatic amino acids |
| Phytic acid_divalent | Phytic acid | Pathway overview |
| Pipecolic acid | Pipecolic acid | Lipid and amino acid metabolism |
| Porphobilinogen | Porphobilinogen | Lipid and amino acid metabolism |
| Pro | Pro | Urea cycle relating metaboloism |
| Propionic acid | Propionic acid | Lipid and amino acid metabolism/ |
| | | BCAA & aromatic amino acid |
| Propionyl CoA_divalent | Propanoyl-CoA | Lipid and amino acid metabolism/ |
| | | BCAA & aromatic amino acids/ |
| | | Nucleotide metabolism |
| PRPP | PRPP | Central carbon metabolism/ |
| | | Nucleotide metabolism |
| Putrescine | Putrescine | Urea cycle relating metaboloism |
| Pyridoxal | Pyridoxal | Metabolism of coenzymes |
| Pyridoxal 5-phosphate | PLP | Metabolism of coenzymes |
| Pyridoxamine | Pyridoxamine | Metabolism of coenzymes |
| Pyridoxamine 5′-phosphate | Pyridoxamine-P | Metabolism of coenzymes |
| Pyridoxine | Pyridoxine | Metabolism of coenzymes |
| Pyruvic acid | Pyruvic acid | Central carbon metabolism/ |
| | | Urea cycle relating metaboloism/ |
| | | Lipid and amino acid metabolism |
| Quinolinic acid | Quinolinic acid | BCAA & aromatic amino acids/ |
| | | Metabolism of coenzymes |
| Riboflavin | Riboflavin | Metabolism of coenzymes |
| Ribose 1-phosphate | R1P | Pathway overview |
| Ribose 5-phosphate | R5P | Central carbon metabolism/ |
| | | Metabolism of coenzymes |
| Ribulose 5-phosphate | Ru5P | Central carbon metabolism |
| Saccharopine | Saccharopine | Lipid and amino acid metabolism |
| S-Adenosylhomocysteine | SAHC | Lipid and amino acid metabolism |
| S-Adenosylmethionine | SAM | Lipid and amino acid metabolism |
| Sarcosine | Sarcosine | Lipid and amino acid metabolism |
| Sedoheptulose 7-phosphate | S7P | Central carbon metabolism |
| Ser | Ser | Lipid and amino acid metabolism |
| Serotonin | Serotonin | BCAA & aromatic amino acids |
| S-Lactoylglutathione | S-Lactoylglutathione | Urea cycle relating metaboloism |
| Spermidine | Spermidine | Urea cycle relating metaboloism |
| Spermine | Spermine | Urea cycle relating metaboloism |
| Succinic acid | Succinic acid | Central carbon metabolism/ |
| | | Urea cycle relating metaboloism |
| Succinic semialdehyde | Succinic semialdehyde | Urea cycle relating metaboloism |
| Succinyl CoA_divalent | SucCoA | Central carbon metabolism |
| Taurine | Taurine | Lipid and amino acid metabolism |
| Taurocholic acid | Taurocholic acid | Lipid and amino acid metabolism |
| Taurocyamine | Taurocyamine | Lipid and amino acid metabolism |
| Thiamine | Thiamine | Metabolism of coenzymes |
| Thiamine diphosphate | ThPP | Metabolism of coenzymes |
| Thiamine phosphate | TMP | Metabolism of coenzymes |
| Thr | Thr | Lipid and amino acid metabolism |
| Thymidine | Thymidine | Nucleotide metabolism |
| Thymine | Thymine | Nucleotide metabolism |
| Trp | Trp | BCAA & aromatic amino acids |
| Tryptamine | Tryptamine | BCAA & aromatic amino acids |
| Tyr | Tyr | BCAA & aromatic amino acids |
| Tyramine | Tyramine | BCAA & aromatic amino acids |
| UDP | UDP | Nucleotide metabolism |
| UDP-glucose | UDP-Glc | Central carbon metabolism |
| UDP-glucuronic acid | UDP-GlcA | Central carbon metabolism |
| UDP-N-acetyglucosamine | UDP-GlcNAc | Central carbon metabolism |
| UMP | UMP | Nucleotide metabolism |
| Uracil | Uracil | Nucleotide metabolism |
| Urea | Urea | Urea cycle relating metaboloism |
| Uric acid | Uric acid | Nucleotide metabolism |
| Uridine | Uridine | Nucleotide metabolism |
| Urocanic acid | Urocanic acid | Urea cycle relating metaboloism |
| UTP | UTP | Nucleotide metabolism |
| Val | Val | BCAA & aromatic amino acids |
| VanillyImandelic acid | VMA | BCAA & aromatic amino acids |
| Xanthine | Xanthine | Nucleotide metabolism |
| Xanthosine | Xanthosine | Nucleotide metabolism |
| Xanthurenic acid | Xanthurenic acid | BCAA & aromatic amino acids |
| XMP | XMP | Nucleotide metabolism |
| XTP | XTP | Nucleotide metabolism |
| Xylulose 5-phosphate | X5P | Central carbon metabolism |
| β -Ala | β-Ala | Urea cycle relating metabolism/ |
| | | Nucleotide metabolism/ |
| | | Metabolismof coenzymes |
| γ-Butyrobetaine | Actinine | Lipid and amino acid metabolism |
| γ-Glu-Cys | g-Glu-Cys | Urea cycle relating metaboloism |

^{†}Metabolites which have been already known about pathway information were listed up. They included metaboites which were not detected in this study.
^{‡}Abbreviated names in Pathway Map.
^{§}Pathway information in the metabolites.

APPENDIX 1-2
Pathway Abbreviations

| Pathway Label^{‡} | Candidates^{†} | Pathway Index^{§} |
|---|---|---|
| 1-Methylhistamine | 1-Methylhistamine | Urea cycle relating metaboloism |
| 1-Methylnicotinamide | 1-Methylnicotinamide | Metabolism of coenzymes |
| 2-Aminoadipic acid | 2-Aminoadipic acid | Lipid and amino acid metabolism |
| 2-Aminophenol | o-Aminophenol | BCAA & aromatic amino acids |
| 2-HBA | 2-Hydroxybutyric acid | Lipid and amino acid metabolism |
| 2-HPAA | o-Hydroxyphenylacetic acid | BCAA & aromatic amino acids |
| 2K3MVA | 3-Methyl-2-oxovaleric acid | Lipid and amino acid metabolism |
| 2-KIV | 2-Oxoisovaleric acid | BCAA & aromatic amino acids |
| 2-OG | 2-Oxoglutaric acid | Central carbon metabolism/Urea |
| | | cycle relating metaboloism |
| 2-Oxoadipic acid | 2-Oxoadipic acid | Lipid and amino acid metabolism |
| 2-Oxobutyric acid | 2-Oxobutyric acid | Lipid and amino acid metabolism |
| 2-Oxoleucine | 4-Methyl-2-oxovaleric acid | Lipid and amino acid metabolism |
| 2-PG | 2-Phosphoglyceric acid | Central carbon metabolism |
| 3.5-DI-Tyr | 3.5-Diiodotyrosine | BCAA & aromatic amino acids |
| 3-Aminoisobutyric acid | 3-Aminoisobutyric acid | BCAA & aromatic amino acids/Nucleotide |
| | | metabolism |
| 3-HBA | 3-Hydroxybutyric acid | Central carbon metabolism/Lipid |
| | | and amino acid metabolism |
| 3-Methoxyanthranilic acid | 3-Methoxyanthranilic acid | BCAA & aromatic amino acids |
| 3-Methoxytyramine | 3-Methoxytyramine | BCAA & aromatic amino acids |
| 3-Methylcrotonyl-CoA | 3-Methylcrotonyl-CoA_divalent | BCAA & aromatic amino acids |
| 3-Methylhistidine | 3-Methylhistidine | Urea cycle relating metaboloism |
| 3-OHAA | 3-Hydroxyanthranilic acid | BCAA & aromatic amino acids |
| 3-OHKY | 3-Hydroxykynurenine | BCAA & aromatic amino acids |
| 3-PG | 3-Phosphoglyceric acid | Central carbon metabolism/Lipid |
| | | and amino acid metabolism |
| 3PSer | O-Phosphoserine | Lipid and amino acid metabolism |
| 3-Ureidopropionic acid | 3-Ureidopropionic acid | Nucleotide metabolism |
| 4-Acetamidobutanoic acid | 4-Acetamidobutanoic acid | Pathway overview |
| 4-GBA | 4-Guanidinobutyric acid | Urea cycle relating metaboloism |
| 4-HPAA | p-Hydroxyphenylacetic acid | BCAA & aromatic amino acids |
| 4-Hydroxyphenylacetaldehyde | 4-Hydroxyphenylacetaldehyde | Pathway overview |
| 4-Pyridoxic acid | 4-Pyridoxic acid | Metabolism of coenzymes |
| 5-ALA | 5-Amino-4-oxovaleric acid | Lipid and amino acid metabolism |
| 5-Hydroxy-IAA | 5-Hydroxyindoleacetic acid | BCAA & aromatic amino acids |
| 5-hydroxylysine | 5-hydroxylysine | Lipid and amino acid metabolism |
| 5-MIAA | 5-Methoxyindoleacetic acid | BCAA & aromatic amino acids |
| 5MOT | 5-Methoxytryptamine | BCAA & aromatic amino acids |
| 5-MHTF | 5-Methyltetrahydrofolic acid | Metabolism of coenzymes |
| 6-PG | 6-Phosphogluconic acid | Central carbon metabolism |
| AAcCoA | Acetoacetyl CoA_divalent | Lipid and amino acid metabolism |
| AcCoA | Acetyl CoA_divalent | Central carbon metabolism/Lipid and |
| | | amino acid metabolism/Metabolism |
| | | of coenzymes |
| Acetanilide | Acetanilide | BCAA & aromatic amino acids |
| Acetoacetic acid | Acetoacetic acid | Central carbon metabolism/Lipid |
| | | and amino acid metabolism |
| Acetylcholine | Acetylcholine | Lipid and amino acid metabolism |
| Actinine | y-Butyrobetaine | Lipid and amino acid metabolism |
| Adenine | Adenine | Nucleotide metabolism |
| Adenosine | Adenosine | Nucleotide metabolism |
| ADP | ADP | Central carbon metabolism/Nucleotide |
| | | metabolism |
| ADP-Rib | ADP-ribose | Central carbon metabolism/Metabolism |
| | | of coenzymes |
| Adrenaline | Adrenaline | BCAA & aromatic amino acids |
| Agmatine | Agmatine | Urea cycle relating metabolosim |
| AICAR | 5-Aminoimidazole-4- | Nucleotide metabolism |
| | carboxamdie ribotide | |
| Ala | Ala | Central carbon metabolism/Urea cycle |
| | | relating metabolosim/BCAA & aromatic |
| | | amino acids |
| ALCAR | O-Acetylcarnitine | Lipid and amino acid metabolism |
| Allantoic acid | Allantoic acid | Pathway overview |
| AMP | AMP | Nucleotide metabolism |
| Anserine | Anserine_divalent | Urea cycle relating metabolosim |
| Anthranilic acid | Anthranilic acid | BCAA & aromatic amino acids |
| AppppA | P¹, P⁴-Di(adenosine-5′) | Nucleotide metabolism |
| | tetraphosphate_divalent | |
| Arg | Arg | Central carbon metabolism/Urea |
| | | cycle relating metabolosim |
| ArgSuccinate | Argininosuccinic acid | Urea cycle relating metabolosim |
| Ascorbate 2-glucoside | Ascorbate 2-glucoside | Metabolism of coenzymes |
| Ascorbate 2-phosphate | Ascorbate 2-phosphate | Metabolism of coenzymes |
| Ascorbate 2-sulfate | Ascorbate 2-sulfate | Metabolism of coenzymes |
| Ascorbic acid | Ascorbic acid | Metabolism of coenzymes |
| Asn | Asn | Urea cycle relating metabolosim |
| Asp | Asp | Central carbon metabolism/Urea cycle |
| | | relating metabolosim/Nucleotide |
| | | metabolism |
| ATP | ATP | Central carbon metabolism/Nucleotide |
| | | metabolism |
| β-Ala | β-Ala | Central carbon metabolism/Nucleotide |
| | | metabolism/Metabolism of coenzymes |
| Betaine | Betaine | Lipid and amino acid metabolism |
| Biotin | Biotin | Metabolism of coenzymes |
| b-Lactate | 3-Hydroxypropionic acid | BCAA & aromatic amino acids |

| Candidates^{†} | Pathway Label^{‡} | Pathway Index^{§} |
|---|---|---|
| BTL | Betaine aldehyde_ + H₂O | Lipid and amino acid metabolism |
| cAMP | cAMP | Nucleotide metabolism |
| Carbamoyl-Asp | N-Carbamoylaspartic acid | Urea cycle relating metaboloism/Nucleotide |
| | | metabolism |
| Carbamoyl-P | Carbamoylphosphate | Urea cycle relating metaboloism |
| Carnitine | Carnitine | Lipid and amino acid metabolism |
| Carnosine | Carnosine | Urea cycle relating metaboloism |
| CDP | CDP | Nucleotide metabolism |
| CDP-choline | CDP-choline | Lipid and amino acid metabolism |
| cGMP | cGMP | Nucleotide metabolism |
| Cholic acid | Cholic acid | Lipid and amino acid metabolism |
| Choline | Choline | Lipid and amino acid metabolism |
| cis-Aconitic acid | cis-Aconitic acid | Central carbon metabolism |
| cis-Hydroxyproline | cis-Hydroxyproline | Urea cycle relating metaboloism |
| Citramalic acid | Citramalic acid | Pathway overview |
| Citric acid | Citric acid | Central carbon metabolism |
| Citrulline | Citrulline | Urea cycle relating metaboloism |
| CMP | CMP | Nucleotide metabolism |
| CMP-NeuNAc | CMP-N-acetylneuraminate | Central carbon metabolism |
| CoA | CoA_divalent | Central carbon metabolism/ |
| | | Metabolism of coenzymes |
| Creatine | Creatine | Urea cycle relating metaboloism |
| Creatinine | Creatinine | Urea cycle relating metaboloism |
| CTP | CTP | Nucleotide metabolism |
| Cys | Cys | Urea cycle relating metaboloism/Lipid |
| | | and amino acid metabolism/Metabolism |
| | | of coenzymes |
| Cys-Gly | Cys-Gly | Urea cycle relating metaboloism |
| Cystathionine | Cystathionine | Lipid and amino acid metabolism |
| Cysteamine | Cysteamine | Lipid and amino acid metabolism |
| Cysteic acid | Cysteic acid | Lipid and amino acid metabolism |
| Cysteinesulfinic acid | Cysteinesulfinic acid | Lipid and amino acid metabolism |
| Cystine | Cystine | Lipid and amino acid metabolism |
| Cytidine | Cytidine | Nucleotide metabolism |
| dAdenosine | 2′-Deoxyadenosine | Nucleotide metabolism |
| dADP | dADP | Nucleotide metabolism |
| dAMP | dAMP | Nucleotide metabolism |
| DAP | 1,3-Diaminopropane | Urea cycle relating metaboloism |
| dATP | dATP | Nucleotide metabolism |
| dCDP | dCDP | Nucleotide metabolism |
| dCMP | dCMP | Nucleotide metabolism |
| dCTP | dCTP | Nucleotide metabolism |
| dCyt | 2′-Deoxycytidine | Nucleotide metabolism |
| Deamido-NAD | Deamido-NAD+ | Metabolism of coenzymes |
| Dephospho-CoA | 3′-Dephospho CoA | Metabolism of coenzymes |
| Desthiobiotin | Desthiobiotin | Metabolism of coenzymes |
| D-F1P | Fructose 1-phosphate | Central carbon metabolism |
| dGDP | dGDP | Nucleotide metabolism |
| dGMP | dGMP | Nucleotide metabolism |
| dGTP | dGTP | Nucleotide metabolism |
| dGuanosine | 2′-Deoxyguanosine | Nucleotide metabolism |
| DHAP | Dihydroxyacetone phosphate | Central carbon metabolism/Lipid |
| | | and amino acid metabolism |
| DHPG | 3,4-Dihydroxyphenylglycol | Pathway overview |
| Dihydrofolic acid | 7,8-Dihydrofolic acid | Metabolism of coenzymes |
| Dihydroorotic acid | Dihydroorotic acid | Nucleotide metabolism |
| Dihydrouracil | Dihydrouracil | Nucleotide metabolism |
| Dimethylbenzimidazole | 5,6-Dimethylbenzimidazole | Metabolism of coenzymes |
| dIMP | dIMP | Nucleotide metabolism |
| Diphosphoglycerate | 2,3-Diphosphoglyceric acid | Central carbon metabolism |
| dITP | dITP | Nucleotide metabolism |
| DMG | N,N-Dimethylglycine | Lipid and amino acid metabolism |
| DOPA | DOPA | BCAA & aromatic amino acids |
| Dopamine | Dopamine | BCAA & aromatic amino acids |
| dTDP | dTDP | Nucleotide metabolism |
| dTMP | dTMP | Nucleotide metabolism |
| dTTP | dTTP | Nucleotide metabolism |
| dUDP | dUDP | Nucleotide metabolism |
| dUMP | dUMP | Nucleotide metabolism |
| dUri | 2′-Deoxyuridine | Nucleotide metabolism |
| dUTP | dUTP | Nucleotide metabolism |
| E4P | Erythrose 4-phosphate | Central carbon metabolism |
| Ergothioneine | Ergothioneine | Pathway overview |
| F1,6P | Fructose 1,6-diphosphate | Central carbon metabolism |
| F6P | Fructose 6-phosphate | Central carbon metabolism |

| Pathway Label^{‡} | Candidates^{†} | Pathway Index^{§} |
|---|---|---|
| FAD | FAD_divalent | Metabolism of coenzymes |
| FMN | FMN | Metabolism of coenzymes |
| Folic acid | Folic acid | Metabolism of coenzymes |
| Formylanthranilate | Formylanthranilic acid | Pathway overview |
| Fumaric acid | Fumaric acid | Central carbon metabolism/Urea |
| | | cycle relating metaboloism |
| G1P | Glucose 1-phosphate | Central carbon metabolism |
| G3P | Glycerol 3-phosphate | Central carbon metabolism/Lipid |
| | | and amino acid metabolism |
| G6P | Glucose 6-phosphate | Central carbon metabolism |
| GABA | GABA | Urea cycle relating metaboloism |
| Gal1P | Galactose 1-phosphate | Central carbon metabolism |
| GAP | Glyceraldehyde 1-phosphate | Central carbon metabolism/Lipid |
| | | and amino acid metabolism |
| GDP | GDP | Nucleotide metabolism |
| GDP-fucose | GDP-fucose | Central carbon metabolism |
| GDP-Man | GDP-mannose | Central carbon metabolism |
| Gensigen | 2,5-Dihydroxybenzoic acid | Pathway overview |
| g-Glu-Cys | γ-Glu-Cys | Urea cycle relating metaboloism |
| Glc-6p | Glucosamine 6-phosphate | Central carbon metabolism |
| GlcNAc | N-Acetylglucosamine | Central carbon metabolism |
| GlcNAc-P | N-Acetylglucosamine 1-phosphate | Central carbon metabolism |
| Gln | Gln | Urea cycle relating metaboloism |
| Glu | Glu | Central carbon metabolism/Urea |
| | | cycle relating metaboloism |
| Glucosamine | Glucosamine | Central carbon metabolism |
| Glucosaminic acid | Glucosaminic acid | Central carbon metabolism |
| Glucuronic acid | Glucuronic acid | Central carbon metabolism |
| Glutaryl-CoA | Glutaryl-CoA | Lipid and amino acid metabolism |
| Gly | Gly | Urea cycle relating metabolism/Lipid |
| | | and amino acid metabolism |
| Glyceric acid | Glyceric acid | Central carbon metabolism/Lipid |
| | | and amino acid metabolism |
| Glycocholic acid | Glycocholic acid | Lipid and amino acid metabolism |
| Glycolic acid | Glycolic acid | Lipid and amino acid metabolism |
| Glyoxylic acid | Glyoxylic acid | Lipid and amino acid metabolism |
| GMP | GMP | Nucleotide metabolism |
| GPCho | Glycerophosphocholine | Lipid and amino acid metabolism |
| GSH | Glutathione (GSH) | Urea cycle relating metaboloism |
| GSSG | Glutathione (GSH)_divalent | Urea cycle relating metaboloism |
| GTP | GTP | Nucleotide metabolism |
| Guanidoacetic acid | Guanidoacetic acid | Urea cycle relating metaboloism |
| Guanine | Guanine | Nucleotide metabolism |
| Guanosine | Guanosine | Nucleotide metabolism |
| His | His | Urea cycle relating metaboloism |
| Histamine | Histamine | Urea cycle relating metaboloism |
| HMG-CoA | HMG CoA_divalent | Lipid and amino acid metabolism |
| Homocysteine | Homocysteine | Lipid and amino acid metabolism |
| HPP | p-Hydroxyphenylpyruvic acid | BCAA & aromatic amino acids |
| HVA | Homovanillic acid | BCAA & aromatic amino acids |
| Hydroxyproline | Hydroxyproline | Urea cycle relating metaboloism |
| Hypotaurine | Hypotaurine | Lipid and amino acid metabolism |
| Hypoxanthine | Hypoxanthine | Nucleotide metabolism |
| IDP | IDP | Nucleotide metabolism |
| Ile | Ile | BCAA & aromatic amino acids |
| Imidazole-4-acetic acid | Imidazole-4-acetic acid | Urea cycle relating metaboloism |
| IMP | IMP | Nucleotide metabolism |
| Indole-3-acetic acid | Indole-3-acetic acid | BCAA & aromatic amino acids |
| Indole-3-acetaldehyde | Indole-3-acetaldehyde | BCAA & aromatic amino acids |
| Inosine | Inosine | Nucleotide metabolism |
| Isobutyryl-CoA | Isobutyryl CoA_divalent | Lipid and amino acid metabolism/BCAA |
| | | & aromatic amino acids |
| Isocitric acid | Isocitric acid | Central carbon metabolism |
| ITP | ITP | Nucleotide metabolism |
| KMTB | 4-Methylthio-2-oxobutyric acid | Lipid and amino acid metabolism |
| Kynurenic acid | Kynurenic acid | BCAA & aromatic amino acids |
| Kynurenine | Kynurenine | BCAA & aromatic amino acids |
| Lactic acid | Lactic acid | Central carbon metabolism/Urea |
| | | cycle relating metaboloism |
| Leu | Leu | BCAA & aromatic amino acids |
| Lys | Lys | Lipid and amino acid metabolism |
| Malic acid | Malic acid | Central carbon metabolism/Urea |
| | | cycle relating metaboloism |
| Malonyl-CoA | Malonyl CoA_divalent | Central carbon metabolism/Lipid |
| | | and amino acid metabolism |
| Man1P | Mannose 1-phosphate | Central carbon metabolism |
| Man6P | Mannose 6-phosphate | Central carbon metabolism |
| ManNAc | N-Acetylmannosamine | Central carbon metabolism |
| Melatonin | Melatonin | BCAA & aromatic amino acids |
| Met | Met | Lipid and amino acid metabolism |
| Methylmalonic acid | Methylmalonic acid | Lipid and amino acid metabolism/BCAA |
| | | & aromatic amino acids |
| MHPG | 3-Methoxy- | BCAA & aromatic amino acids |
| | 4-hydroxyphenylethyleneglycol | |
| MIA | 1-Methyl-4-imidazoleacetic acid | Urea cycle relating metaboloism |
| MIT | 3-Iodotyrosine | BCAA & aromatic amino acids |
| MTA | 5′-Deoxy-5′-methylthioadenosine | Urea cycle relating metaboloism |
| N-Acetylaspartic acid | N-Acetylaspartic acid | Urea cycle relating metaboloism |
| N-Acetylputrescine | N-Acetylputrescine | Urea cycle relating metaboloism |
| N-AcGlcNP | N-Acetylglucosamine 6-phosphate | Central carbon metabolism |
| N-AcGlu | N-Acetylglutamic acid | Urea cycle relating metaboloism |
| N-AcOrn | N-Acetylornithine | Urea cycle relating metaboloism |
| NAD⁺ | NAD⁺ | Central carbon metabolism/Metabolism |
| | | of coenzymes |
| NADH | NADH | Central carbon metabolism/Metabolism |
| | | of coenzymes |
| NADP⁺ | NADP⁺ | Central carbon metabolism/Metabolism |
| | | of coenzymes |
| NADPH | NADPH_divalent | Central carbon metabolism/Metabolism |
| | | of coenzymes |
| NeuNAc | N-Acetylneuraminic acid | Central carbon metabolism |
| N-Formyl aspartic acid | N-Formylaspartic acid | Urea cycle relating metaboloism |
| Nicotinamide | Nicotinamide | Metabolism of coenzymes |
| Nicotinic acid | Nicotinic acid | Metabolism of coenzymes |
| NicRN | NMN | Metabolism of coenzymes |
| N-Methylserotonin | N-Methylserotonin | Pathway overview |
| N-Methyltryptamine | N-Methyltryptamine | BCAA & aromatic amino acids |
| N-Methyltyramine | N-Methyltyramine | BCAA & aromatic amino acids |
| Noradrenaline | Noradrenaline | BCAA & aromatic amino acids |
| Normetanephrine | Normetanephrine | Pathway overview |
| Ornithine | Ornithine | Urea cycle relating metaboloism |
| Orotic acid | Orotic acid | Nucleotide metabolism |
| Orotidine5′P | Orotidine 5′-monophosphate | Nucleotide metabolism |
| Oxoproline | 5-Oxoproline | Urea cycle relating metaboloism |
| P5C | 1-Pyrroline 5-carboxylic acid | Urea cycle relating metaboloism |
| Pantothenic acid | Pantothenic acid | Metabolism of coenzymes |
| PEP | Phosphoenolpyruvic acid | Central carbon metabolism |
| Phe | Phe | BCAA & aromatic amino acids |
| Phenaceturic acid | Phenaceturic acid | BCAA & aromatic amino acids |
| Phenylethylamine | 2-Phenylethylamine | BCAA & aromatic amino acids |
| Phenylpyruvate | Phenylpyruvic acid | BCAA & aromatic amino acids |
| Phosphocreatine | Phosphocreatine | Urea cycle relating metaboloism |
| Phosphorylcholine | Phosphorylcholine | Lipid and amino acid metabolism |
| Phytic acid | Phytic acid_divalent | Pathway overview |
| Pipecolic acid | Pipecolic acid | Lipid and amino acid metabolism |
| PLP | Pyridoxal 5-phosphate | Metabolism of coenzymes |
| Porphobilinogen | Porphobilinogen | Lipid and amino acid metabolism |
| Pretonine | 5-Hydroxytryptophan | BCAA & aromatic amino acids |
| Pro | Pro | Urea cycle relating metaboloism |
| Phytic acid | Phytic acid_divalent | Pathway overview |
| Pipecolic acid | Pipecolic acid | Lipid and amino acid metabolism |
| PLP | Pyridoxal 5-phosphate | Metabolism of coenzymes |
| Porphobilinogen | Porphobilinogen | Lipid and amino acid metabolism |
| Pretonine | 5-Hydroxytryptophan | BCAA & aromatic amino acids |
| Pro | Pro | Urea cycle relating metaboloism |
| Propanoyl-CoA | Propionyl CoA_divalent | Lipid and amino acid metabolism/BCAA & |
| | | aromatic amino acids/Nucleotide metabolism |
| Propionic acid | Propionic acid | Lipid and amino acid metabolism/BCAA & |
| | | aromatic amino acids |
| PRPP | PRPP | Central carbon metabolism/Nucleotide |
| | | metabolism |
| Putrescine | Putrescine | Urea cycle relating metaboloism |
| Pyridoxal | Pyridoxal | Metabolism of coenzymes |
| Pyridoxamine | Pyridoxamine | Metabolism of coenzymes |
| Pyridoxamine-P | Pyridoxamine 5′-phosphate | Metabolism of coenzymes |
| Pyridoxine | Pyridoxine | Metabolism of coenzymes |
| Pyruvic acid | Pyruvic acid | Central carbon metabolism/Urea cycle |
| | | relating metaboloism/Lipid and amino |
| | | acid metabolism |
| Quinolinic acid | Quinolinic acid | BCAA & aromatic amino acids/Metabolism |
| | | of coenzymes |
| R1P | Ribose 1-phosphate | Pathway overview |
| R5P | Ribose 5-phosphate | Central carbon metabolism/Metabolism of |
| | | coenzymes |
| Riboflavin | Riboflavin | Metabolism of coenzymes |
| Ru5P | Ribulose 5-phosphate | Central carbon metabolism |
| S7P | Sedoheptulose 7-phosphate | Central carbon metabolism |
| Saccharopine | Saccharopine | Lipid and amino acid metabolism |
| SAHC | S-Adenosylhomocysteine | Lipid and amino acid metabolism |
| SAM | S-Adenosylmethionine | Lipid and amino acid metabolism |
| Sarcosine | Sarcosine | Lipid and amino acid metabolism |
| Ser | Ser | Lipid and amino acid metabolism |
| Serotonin | Serotonin | BCAA & aromatic amino acids |
| S-Lactoylglutathione | S-Lactoylglutathione | Urea cycle relating metaboloism |
| Spermidine | Spermidine | Urea cycle relating metaboloism |
| Spermine | Spermine | Urea cycle relating metaboloism |
| Succinic acid | Succinic acid | Central carbon metabolism/Urea cycle |
| | | relating metabolism |
| Succinic semialdehyde | Succinic semialdehyde | Urea cycle relating metaboloism |
| Succinyl AMP | Adenylosuccinic acid | Nucleotide metabolism |
| SucCoA | Succinyl CoA_divalent | Central carbon metabolism |
| T3 | 3,3′,5-Triiodothyronine | BCAA & aromatic amino acids |
| Taurine | Taurine | Lipid and amino acid metabolism |
| Taurocholic acid | Taurocholic acid | Lipid and amino acid metabolism |
| Taurocyamine | Taurocyamine | Lipid and amino acid metabolism |
| TDP-Glc | dTDP-glucose | Pathway overview |
| Thiamine | Thiamine | Metabolism of coenzymes |
| ThPP | Thamine diphosphate | Metabolism of coenzymes |
| Thr | Thr | Lipid and amino acid metabolism |
| Thymidine | Thymidine | Nucleotide metabolism |
| Thymine | Thymine | Nucleotide metabolism |
| TMP | Thamine phosphate | Metabolism of coenzymes |
| Trimethyllysine | N⁶,N⁶,N⁶-Trimethyllysine | Lipid and amino acid metabolism |
| Trp | Trp | BCAA & aromatic amino acids |
| Tryptamine | Tryptamine | BCAA & aromatic amino acids |
| Tyr | Tyr | BCAA & aromatic amino acids |
| Tyramine | Tyramine | BCAA & aromatic amino acids |
| UDP | UDP | Nucleotide metabolism |
| UDP-Glc | UDP-glucose | Central carbon metabolism |
| UDP-GlcA | UDP-glucuronic acid | Central carbon metabolism |
| UDP-GlcNAc | UDP-N-acetyglucosamine | Central carbon metabolism |
| UMP | UMP | Nucleotide metabolism |
| Uracil | Uracil | Nucleotide metabolism |
| Urea | Urea | Urea cycle relating metaboloism |
| Uric acid | Uric acid | Nucleotide metabolism |
| Uridine | Uridine | Nucleotide metabolism |
| Urocanic acid | Urocanic acid | Urea cycle relating metaboloism |
| UTP | UTP | Nucleotide metabolism |
| Val | Val | BCAA & aromatic amino acids |
| VMA | Vanillylmandelic acid | BCAA & aromatic amino acids |
| X5P | Xylulose 5-phosphate | Central carbon metabolism |
| Xanthine | Xanthine | Nucleotide metabolism |
| Xanthosine | Xanthosine | Nucleotide metabolism |
| Xanthurenic acid | Xanthurenic acid | BCAA & aromatic amino acids |
| XMP | XMP | Nucleotide metabolism |
| XTP | XTP | Nucleotide metabolism |

^{‡}Abbreviated names in Pathway Map.
^{†}Metabolites which have been already known about pathway information were listed up. They included metaboites which were not detected in this study.
^{§}Pathway information in the metabolites.

APPENDIX 2
Known-Unknown Peaks
The “known-unknown” peaks with out annotation based on the chemical
standards are shown in the label of “XA~~~~/XC~~~~” in result tables.
Among them, several peaks which have been detected from a variety of
biological samples are listed in Appendix 2.

| | | | Candidate compounds | | |
|---|---|---|---|---|---|
| HMT ID | Peak ID | Mode^{†} | mass^{‡} | PubChem database | HMDB database |
| M90001 | XA0001 | Anion | 107.998 | | |
| M90002 | XA0002 | Anion | 111.993 | 75795 | |
| M90003 | XA0003 | Anion | 125.999 | 7866 | |
| M90004 | XA0004 | Anion | 145.038 | 440726; 48 | HMDB01552 |
| M90005 | XA0005 | Anion | 150.052 | 11389478; 125409; 135191; | HMDB00098; HMDB00283; |
| | | | | 439195; 439203; 439204; | HMDB00366; HMDB00621; |
| | | | | 439205; 439240; 439245; | HMDB00646; HMDB00751; |
| | | | | 439508; 439678; 439731; | HMDB01644; HMDB03371; |
| | | | | 439764; 440921; 441474; | HMDB12194; HMDB12325 |
| | | | | 441481; 441482; 447347; | |
| | | | | 5460157; 5460291; 5779; 6027; | |
| | | | | 619; 6902 | |
| M90006 | XA0006 | Anion | 150.067 | | |
| M90007 | XA0007 | Anion | 152.014 | | |
| M90008 | XA0008 | Anion | 154.003 | 1034; 150865; 440171 | |
| M90009 | XA0009 | Anion | 154.026 | 19; 3469; 4696; 72 | HMDB00152; HMDB00397; |
| | | | | | HMDB01856 |
| M90010 | XA0010 | Anion | 155.035 | 439436; 440231; 440233 | |
| M90011 | XA0011 | Anion | 165.019 | | |
| M90012 | XA0012 | Anion | 167.025 | | HMDB06462 |
| M90013 | XA0013 | Anion | 173.999 | 4765; 74426 | |
| M90014 | XA0014 | Anion | 174.016 | 440667; 444212; 4784 | HMDB00958; HMDB01264 |
| M90015 | XA0015 | Anion | 174.125 | | |
| M90016 | XA0016 | Anion | 186.029 | | |
| M90017 | XA0017 | Anion | 187.121 | 173; 5282047 | |
| M90018 | XA0018 | Anion | 188.115 | 440139; 92832; 92843; 92907 | HMDB00206; HMDB00446; |
| | | | | | HMDB00759 |
| M90019 | XA0019 | Anion | 192.027 | 102287; 36681; 439290; | HMDB01874; HMDB05971; |
| | | | | 440165; 440390; 447805; | HMDB06511 |
| | | | | 5318532 | |
| M90020 | XA0020 | Anion | 197.036 | 3082376 | |
| M90021 | XA0021 | Anion | 200.008 | 146355; 439910; 5206; | |
| | | | | 5459897 | |
| M90022 | XA0022 | Anion | 200.045 | | |
| M90023 | XA0023 | Anion | 208.021 | 6812; 8420 | |
| M90024 | XA0024 | Anion | 217.104 | | |
| M90025 | XA0025 | Anion | 224.014 | | |
| M90026 | XA0026 | Anion | 225.030 | | |
| M90027 | XA0027 | Anion | 228.208 | 11005 | HMDB00806; HMDB02221 |
| M90028 | XA0028 | Anion | 231.537 | | |
| M90029 | XA0029 | Anion | 237.030 | | |
| M90030 | XA0030 | Anion | 238.068 | 119228; 439706 | |
| M90031 | XA0031 | Anion | 240.099 | | |
| M90032 | XA0032 | Anion | 240.135 | | |
| M90033 | XA0033 | Anion | 243.087 | 53297342; 6175; 6253 | HMDB00089 |
| M90034 | XA0034 | Anion | 243.184 | | |
| M90035 | XA0035 | Anion | 255.988 | 54675759 | |
| M90036 | XA0036 | Anion | 255.988 | 54675759 | |
| M90037 | XA0037 | Anion | 274.014 | | |
| M90038 | XA0038 | Anion | 274.045 | 15942876 | |
| M90039 | XA0039 | Anion | 287.067 | | |
| M90040 | XA0040 | Anion | 290.171 | | |
| M90041 | XA0041 | Anion | 303.540 | | |
| M90042 | XA0042 | Anion | 309.120 | | |
| M90043 | XA0043 | Anion | 310.513 | | |
| M90044 | XA0044 | Anion | 312.229 | 11954062; 18172; 5280720; | HMDB03871; HMDB04706; |
| | | | | 5281026; 5283016; 5460412; | HMDB06940; HMDB10201; |
| | | | | 6438758; 9548877 | HMDB10208; HMDB10221 |
| M90045 | XA0045 | Anion | 321.069 | | |
| M90046 | XA0046 | Anion | 326.526 | | |
| M90047 | XA0047 | Anion | 333.037 | | |
| M90048 | XA0048 | Anion | 334.066 | 440418; 44224013; 442419; | HMDB11649 |
| | | | | 45480545; 90658884 | |
| M90049 | XA0049 | Anion | 337.023 | | |
| M90050 | XA0050 | Anion | 339.073 | | |
| M90051 | XA0051 | Anion | 339.995 | 10267; 105021; 125004; | HMDB00968; HMDB01047; |
| | | | | 128419; 3036654; 439444; | HMDB03514; HMDB06234; |
| | | | | 440117; 440211; 82400 | HMDB06235; HMDB06872 |
| M90052 | XA0052 | Anion | 343.093 | 10925943 | |
| M90053 | XA0053 | Anion | 353.003 | | |
| M90054 | XA0054 | Anion | 368.163 | 12594; 240071 | HMDB01032; HMDB02833 |
| M90055 | XA0055 | Anion | 370.006 | 164735; 46906053 | |
| M90056 | XA0056 | Anion | 383.052 | | |
| M90057 | XA0057 | Anion | 397.121 | | |
| M90058 | XA0058 | Anion | 400.016 | | |
| M90059 | XA0059 | Anion | 421.027 | | |
| M90060 | XA0060 | Anion | 422.012 | | |
| M90061 | XA0061 | Anion | 423.094 | | |
| M90062 | XA0062 | Anion | 424.036 | | |
| M90063 | XA0063 | Anion | 425.586 | | |
| M90064 | XA0064 | Anion | 437.972 | | |
| M90065 | XA0065 | Anion | 446.060 | 123727 | HMDB01564 |
| M90066 | XA0066 | Anion | 448.141 | 73607 | |
| M90067 | XA0067 | Anion | 495.189 | | |
| M90068 | XA0068 | Anion | 536.044 | 23724459; 23724466; 439536; | HMDB01018; HMDB12301; |
| | | | | 46174047 | HMDB12303 |
| M90069 | XA0069 | Anion | 536.092 | | |
| M90070 | XA0070 | Anion | 537.076 | 165130 | |
| M90071 | XA0071 | Anion | 542.274 | | HMDB10320 |
| M90072 | XA0072 | Anion | 548.129 | | |
| M90073 | XA0073 | Anion | 633.213 | | HMDB00825; HMDB06569 |
| M90074 | XA0074 | Anion | 745.093 | 5884 | HMDB00221 |
| M90075 | XA0075 | Anion | 747.024 | | |
| M90076 | XA0076 | Anion | 767.117 | 87642 | HMDB01423 |
| M90077 | XA0077 | Anion | 785.160 | 643975 | HMDB01248 |
| M90078 | XA0078 | Anion | 841.053 | | |
| M90079 | XC0001 | Cation | 71.073 | 443732 | |
| M90080 | XC0002 | Cation | 73.053 | 215; 6228; 67180; 75 | HMDB01106; HMDB01888; |
| | | | | | HMDB02134 |
| M90081 | XC0003 | Cation | 89.083 | | |
| M90082 | XC0004 | Cation | 89.084 | | |
| M90083 | XC0005 | Cation | 99.043 | | |
| M90084 | XC0006 | Cation | 103.073 | | |
| M90085 | XC0007 | Cation | 108.571 | | |
| M90086 | XC0008 | Cation | 112.012 | | |
| M90087 | XC0009 | Cation | 113.053 | | |
| M90088 | XC0010 | Cation | 114.078 | | HMDB00323 |
| M90089 | XC0011 | Cation | 115.099 | | |
| M90090 | XC0012 | Cation | 116.094 | 439358 | HMDB12176 |
| M90091 | XC0013 | Cation | 120.060 | | |
| M90092 | XC0014 | Cation | 122.586 | | |
| M90093 | XC0015 | Cation | 125.047 | 194461; 24892813; 3017497; | |
| | | | | 4362; 5460445 | |
| M90094 | XC0016 | Cation | 128.058 | 440769; 440770; 93556 | HMDB00079 |
| M90095 | XC0017 | Cation | 129.089 | 559 | |
| M90096 | XC0018 | Cation | 129.594 | | |
| M90097 | XC0019 | Cation | 130.566 | | |
| M90098 | XC0020 | Cation | 133.036 | 5960; 83887 | HMDB11753 |
| M90099 | XC0021 | Cation | 133.072 | | |
| M90100 | XC0022 | Cation | 133.073 | | |
| M90101 | XC0023 | Cation | 133.073 | | |
| M90102 | XC0024 | Cation | 133.109 | | |
| M90103 | XC0025 | Cation | 137.573 | | |
| M90104 | XC0026 | Cation | 137.574 | | |
| M90105 | XC0027 | Cation | 142.110 | | |
| M90106 | XC0028 | Cation | 143.094 | 115244; 5462194 | |
| M90107 | XC0029 | Cation | 143.094 | 115244; 5462194 | |
| M90108 | XC0030 | Cation | 144.569 | | |
| M90109 | XC0031 | Cation | 145.073 | 160603; 18189; 439954; | HMDB00730; HMDB00808; |
| | | | | 440077; 440805 | HMDB01263; HMDB03681; |
| | | | | | HMDB12131; HMDB12151 |
| M90110 | XC0032 | Cation | 147.034 | 440159 | |
| M90111 | XC0033 | Cation | 151.029 | | |
| M90112 | XC0034 | Cation | 151.576 | | |
| M90113 | XC0035 | Cation | 157.109 | 442645; 4479243 | |
| M90114 | XC0036 | Cation | 160.084 | 439925; 441021 | HMDB03459 |
| | | | | 24906320; 439377; 439389; | |
| M90115 | XC0037 | Cation | 161.068 | 439943; 440550; 440959; | |
| | | | | 46173947; 92136 | |
| M90116 | XC0038 | Cation | 170.068 | | |
| M90117 | XC0039 | Cation | 172.047 | 656724; 782 | HMDB01212 |
| M90118 | XC0040 | Cation | 173.079 | | HMDB04225 |
| M90119 | XC0041 | Cation | 175.028 | | |
| M90120 | XC0042 | Cation | 175.119 | | |
| M90121 | XC0043 | Cation | 178.120 | | |
| M90122 | XC0044 | Cation | 185.104 | 443003; 443845; 5281740 | HMDB06348; HMDB06548 |
| M90123 | XC0045 | Cation | 190.007 | | |
| M90124 | XC0046 | Cation | 190.057 | 121396; 441441 | HMDB11165 |
| M90125 | XC0047 | Cation | 190.094 | 439283; 99290 | HMDB01370 |
| M90126 | XC0048 | Cation | 190.130 | | |
| M90127 | XC0049 | Cation | 191.041 | 27661; 443054; 46173773; | |
| | | | | 8758 | |
| M90128 | XC0050 | Cation | 192.059 | | |
| M90129 | XC0051 | Cation | 193.040 | | |
| M90130 | XC0052 | Cation | 197.057 | 440214 | |
| M90131 | XC0053 | Cation | 203.125 | | |
| M90132 | XC0054 | Cation | 204.073 | 26879 | HMDB11162; HMDB11667 |
| M90133 | XC0055 | Cation | 204.074 | | HMDB11162; HMDB11667 |
| M90134 | XC0056 | Cation | 204.110 | 128597; 128888; 5799 | |
| M90135 | XC0057 | Cation | 204.146 | | |
| M90136 | XC0058 | Cation | 208.051 | 5281921; 6763; 6780 | |
| M90137 | XC0059 | Cation | 212.115 | 2479 | HMDB11180 |
| M90138 | XC0060 | Cation | 216.073 | 46173889 | |
| M90139 | XC0061 | Cation | 217.130 | 107738 | HMDB00824 |
| M90140 | XC0062 | Cation | 218.089 | 151284 | HMDB03764; HMDB06248 |
| M90141 | XC0063 | Cation | 218.125 | 193187 | |
| M90142 | XC0064 | Cation | 220.069 | | HMDB11168 |
| M90143 | XC0065 | Cation | 220.083 | 144; 439280; 442551 | HMDB00472 |
| M90144 | XC0066 | Cation | 221.071 | | |
| M90145 | XC0067 | Cation | 223.104 | | |
| M90146 | XC0068 | Cation | 225.147 | | |
| M90147 | XC0069 | Cation | 228.121 | 441123 | |
| M90148 | XC0070 | Cation | 228.146 | | HMDB11174; HMDB11175 |
| M90149 | XC0071 | Cation | 233.172 | | HMDB11140 |
| M90150 | XC0072 | Cation | 234.084 | | HMDB11169 |
| M90151 | XC0073 | Cation | 234.084 | | HMDB11169 |
| M90152 | XC0074 | Cation | 236.082 | | |
| M90153 | XC0075 | Cation | 237.084 | 128973; 2380; 439921; 440036; | HMDB00238; HMDB00468; |
| | | | | 5460401; 65253 | HMDB00633; HMDB00817; |
| | | | | | HMDB01195; HMDB02263 |
| M90154 | XC0076 | Cation | 240.146 | 4845; 49787007 | |
| M90155 | XC0077 | Cation | 241.632 | | |
| M90156 | XC0078 | Cation | 242.175 | | |
| M90157 | XC0079 | Cation | 245.122 | | |
| M90158 | XC0080 | Cation | 246.120 | | HMDB11166; HMDB11172 |
| M90159 | XC0081 | Cation | 246.120 | | HMDB11166; HMDB11172 |
| M90160 | XC0082 | Cation | 247.081 | | |
| M90161 | XC0083 | Cation | 247.140 | | HMDB13127 |
| M90162 | XC0084 | Cation | 248.063 | 2955 | HMDB11163 |
| M90163 | XC0085 | Cation | 248.100 | | |
| M90164 | XC0086 | Cation | 249.084 | 1076 | HMDB01526; HMDB06878 |
| M90165 | XC0087 | Cation | 253.152 | | |
| M90166 | XC0088 | Cation | 254.038 | 68134 | |
| M90167 | XC0089 | Cation | 254.089 | 10400039; 9921310 | |
| M90168 | XC0090 | Cation | 255.073 | | |
| M90169 | XC0091 | Cation | 255.074 | | |
| M90170 | XC0092 | Cation | 256.139 | | |
| M90171 | XC0093 | Cation | 257.198 | | |
| M90172 | XC0094 | Cation | 258.084 | 440569; 65049 | HMDB00884; HMDB02331; |
| | | | | | HMDB04813 |
| M90173 | XC0095 | Cation | 258.132 | | |
| M90174 | XC0096 | Cation | 260.136 | 10306 | HMDB11170; HMDB11171 |
| M90175 | XC0097 | Cation | 261.096 | | |
| M90176 | XC0098 | Cation | 261.120 | 181804; 441467; 442866 | HMDB13133 |
| M90177 | XC0099 | Cation | 261.131 | 4098 | HMDB02248; HMDB04985; |
| | | | | | HMDB04987 |
| M90178 | XC0100 | Cation | 261.131 | 4098 | HMDB02248; HMDB04985; |
| | | | | | HMDB04987 |
| M90179 | XC0101 | Cation | 261.156 | | |
| M90180 | XC0102 | Cation | 262.079 | | HMDB11164 |
| M90181 | XC0103 | Cation | 265.115 | 168948 | |
| M90182 | XC0104 | Cation | 267.094 | 107795; 35370; 441037 | HMDB00085; HMDB00830 |
| M90183 | XC0105 | Cation | 268.116 | 439693 | |
| M90184 | XC0106 | Cation | 270.095 | 126220 | |
| M90185 | XC0107 | Cation | 275.110 | 150914; 25137932 | HMDB05766; HMDB11738 |
| M90186 | XC0108 | Cation | 275.135 | | HMDB13130 |
| M90187 | XC0109 | Cation | 276.096 | 69925; 9117; 92865 | HMDB11737 |
| M90188 | XC0110 | Cation | 277.564 | | |
| M90189 | XC0111 | Cation | 278.093 | | |
| M90190 | XC0112 | Cation | 279.130 | | |
| M90191 | XC0113 | Cation | 281.110 | 73317 | HMDB04044; HMDB04326; |
| | | | | | HMDB06023 |
| M90192 | XC0114 | Cation | 284.110 | 25447 | |
| M90193 | XC0115 | Cation | 287.057 | 128861; 441648; 444150; | |
| | | | | 6842999 | |
| M90194 | XC0116 | Cation | 289.151 | | HMDB00552 |
| M90195 | XC0117 | Cation | 293.146 | | |
| M90196 | XC0118 | Cation | 294.105 | 440002 | |
| M90197 | XC0119 | Cation | 294.141 | | |
| M90198 | XC0120 | Cation | 297.044 | | HMDB00709 |
| M90199 | XC0121 | Cation | 297.178 | | |
| M90200 | XC0122 | Cation | 302.137 | | |
| M90201 | XC0123 | Cation | 305.738 | | |
| M90202 | XC0124 | Cation | 308.120 | | |
| M90203 | XC0125 | Cation | 308.120 | | |
| M90204 | XC0126 | Cation | 309.104 | 439197; 440038 | HMDB00230; HMDB00773 |
| M90205 | XC0127 | Cation | 310.114 | | HMDB11741 |
| M90206 | XC0128 | Cation | 311.122 | | HMDB01961; HMDB04824 |
| M90207 | XC0129 | Cation | 319.081 | | |
| M90208 | XC0130 | Cation | 321.098 | 115260; 440380 | |
| M90209 | XC0131 | Cation | 322.136 | | |
| M90210 | XC0132 | Cation | 324.152 | 46174023 | HMDB00600 |
| M90211 | XC0133 | Cation | 327.130 | | |
| M90212 | XC0134 | Cation | 335.132 | 123826 | HMDB00489 |
| M90213 | XC0135 | Cation | 336.164 | | |
| M90214 | XC0136 | Cation | 337.092 | 447123; 5360043 | HMDB04662 |
| M90215 | XC0137 | Cation | 349.093 | 11954074; 440596 | |
| M90216 | XC0138 | Cation | 366.141 | | |
| M90217 | XC0139 | Cation | 383.106 | 23724526 | HMDB00912 |
| M90218 | XC0140 | Cation | 387.101 | | |
| M90219 | XC0141 | Cation | 388.123 | 50909833 | |
| M90220 | XC0142 | Cation | 428.141 | | |
| M90221 | XC0143 | Cation | 469.136 | | |

^{†}Molecular ions with positive and negative charge are measured in Cation and Anion Mode, respectively
^{‡}Predicted mass value was calculated as mono-valent ion.

APPENDIX 3
Metabolites Detected
Information
Table 7 “Putative Metabolites”

Peak ID consists of analysis mode and number. The alphabets shows measurement mode;
Cation (C) and Anion (A) mode.
Putative metabolites listed in “Compound name” were assigned on the basis of m/z and MT.
Those listed in “PubChem ID/HMDB ID/peptide” were assigned on the basis of m/z only.
“N.D.” and “N.A” represent “Not Detected” and “Not Available”, respectively.
“Ratio” was calculated between two indicated groups (left: numerator, right: dominator).
“p-value” was calculated on the basis of t-test.
The information about each result was indicated under the table.

TABLE 7

| | | Relative Area | | | | Comparative Analysis | |
|---|---|---|---|---|---|---|---|
| | HMT DB^{†} | Control | | Treatment | | Control vs Treatment | |
| ID | Compound name | Mean | S.D. | Mean | S.D. | Ratio ^{¶} | p-value ^{∥} |

Putative Metabolites (1)

| | | | | | | | |
|---|---|---|---|---|---|---|---|
| C_0056 | 1-Methyl-4-imidazoleacetic acid | 9.7E−05 | N.A. | 1.2E−04 | 3.5E−05 | 0.8 | N.A. |
| C_0124 | 1-Methyladenosine | 5.8E−05 | 3.4E−06 | 5.2E−05 | 4.4E−06 | 1.1 | 0.027 * |
| C_0079 | 1-Methylhistidine | 2.2E−03 | 5.4E−04 | 2.1E−03 | 4.4E−04 | 1.0 | 0.880 |
| | 3-Methylhistidine | | | | | | |
| C_0051 | 1-Methylnicotinamide | 2.9E−04 | 1.4E−04 | 2.4E−04 | 1.2E−04 | 1.2 | 0.563 |
| C_0057 | 1H-Imidazole-4-propionic acid | 1.2E−04 | 2.3E−05 | 1.0E−04 | 1.9E−05 | 1.2 | 0.489 |
| C_0108 | 2′-Deoxycytidine | 2.1E−04 | 1.9E−05 | 2.0E−04 | 1.4E−05 | 1.1 | 0.359 |
| C_0109 | 2′-Deoxyuridine | 4.2E−04 | 8.2E−05 | 4.1E−04 | 8.8E−05 | 1.0 | 0.818 |
| C_0011 | 2-Aminoisobutyric acid | 2.2E−03 | 5.8E−04 | 2.0E−03 | 5.1E−04 | 1.1 | 0.732 |
| | 2-Aminobutyric acid | | | | | | |
| A_0025 | 2-Hydroxy-4-methylvaleric acid | 4.0E−04 | 3.4E−05 | 2.7E−04 | 1.1E−04 | 1.5 | 0.105 |
| A_0008 | 2-Hydroxybutyric acid | 2.6E−03 | 9.6E−04 | 2.4E−03 | 4.7E−04 | 1.1 | 0.720 |
| A_0018 | 2-Hydroxyvaleric acid | 1.1E−03 | 6.5E−04 | 9.9E−04 | 6.4E−04 | 1.1 | 0.825 |
| A_0032 | 2-Oxoglutaric acid | 4.5E−03 | 2.7E−03 | 6.8E−03 | 3.3E−03 | 0.7 | 0.276 |
| A_0013 | 2-Oxoisovaleric acid | 1.1E−03 | 1.9E−04 | 9.2E−04 | 1.1E−04 | 1.2 | 0.224 |
| A_0034 | 3-(4-Hydroxyphenyl)propionic acid | 3.1E−04 | 4.8E−05 | 2.5E−04 | 8.0E−05 | 1.2 | 0.238 |
| A_0009 | 3-Hydroxybutyric acid | 2.8E−02 | 1.6E−02 | 1.6E−02 | 8.5E−03 | 1.7 | 0.248 |
| A_0067 | 3-Indoxylsulfuric acid | 1.9E−03 | 8.2E−04 | 2.5E−03 | 1.2E−03 | 0.8 | 0.387 |
| A_0024 | 3-Ureidopropionic acid | 2.2E−04 | 6.8E−05 | 2.8E−04 | 1.2E−04 | 0.8 | 0.355 |
| A_0031 | 4-Acetamidobutanoic acid | 4.2E−04 | 2.1E−04 | 2.9E−04 | 8.7E−05 | 1.5 | 0.294 |
| A_0021 | 4-Methyl-2-oxovaleric acid | 2.8E−03 | 6.9E−04 | 2.6E−03 | 3.2E−04 | 1.1 | 0.682 |
| | 3-Methyl-2-oxovaleric acid | | | | | | |
| C_0025 | 5-Aminovaleric acid | 8.7E−04 | N.A. | 6.7E−04 | N.A. | 1.3 | N.A. |
| C_0074 | 5-Hydroxylysine | 1.7E−04 | 7.1E−05 | 2.1E−04 | 4.9E−05 | 0.8 | 0.292 |
| C_0104 | 5-Hydroxytryptophan | 8.2E−05 | 1.9E−05 | 7.9E−05 | 1.4E−05 | 1.0 | 0.775 |
| A_0062 | 5-Methoxyindoleacetic acid | 1.8E−04 | 2.9E−05 | 1.9E−04 | 4.4E−05 | 0.9 | 0.728 |
| A_0020 | 5-Oxoproline | 6.6E−04 | 8.4E−05 | 6.0E−04 | 1.9E−04 | 1.1 | 0.511 |
| C_0043 | 6-Aminohexanoic acid | 2.9E−04 | 7.5E−05 | 1.9E−04 | N.A. | 1.5 | N.A. |
| C_0112 | 7,8-Dihydrobiopterin | 4.8E−05 | 5.2E−06 | 5.6E−05 | 1.2E−05 | 0.9 | 0.543 |
| A_0006 | Acetoacetic acid | 3.6E−04 | 1.9E−04 | 2.5E−04 | 4.3E−05 | 1.4 | 0.430 |
| C_0122 | Adenosine | 4.1E−05 | N.A. | 8.3E−05 | 6.6E−05 | 0.5 | N.A. |
| A_0097 | ADP | 1.2E−04 | 2.8E−05 | 5.6E−04 | 1.1E−03 | 0.2 | 0.404 |
| A_0107 | ADP-ribose | 9.4E−05 | 2.2E−05 | 1.9E−04 | 7.8E−05 | 0.5 | 0.319 |
| C_0007 | Ala | 7.3E−02 | 8.7E−03 | 9.1E−02 | 1.9E−02 | 0.8 | 0.076 |
| C_0003 | Aminoacetone | 1.5E−03 | 2.4E−04 | 1.7E−03 | 2.5E−04 | 0.8 | 0.124 |
| A_0086 | AMP | 3.5E−04 | 1.2E−04 | 5.3E−04 | 6.9E−04 | 0.7 | 0.599 |
| C_0030 | Anserine_divalent | 3.0E−04 | 1.3E−04 | 3.1E−04 | 1.3E−04 | 1.0 | 0.924 |
| C_0081 | Arg | 2.2E−02 | 2.3E−03 | 2.8E−02 | 5.1E−03 | 0.8 | 0.065 |
| C_0127 | Argininosuccinic acid | 9.2E−05 | 2.2E−05 | 8.7E−05 | 1.3E−05 | 1.0 | 0.748 |
| C_0044 | Asn | 6.7E−03 | 3.4E−03 | 1.0E−02 | 5.8E−03 | 0.7 | 0.256 |
| C_0047 | Asp | 1.4E−03 | 3.6E−04 | 1.9E−03 | 4.8E−04 | 0.8 | 0.137 |
| A_0104 | ATP | 3.0E−04 | 4.6E−05 | 1.6E−03 | 2.3E−03 | 0.2 | 0.353 |
| C_0026 | Betaine | 2.6E−02 | 7.5E−03 | 2.8E−02 | 1.3E−02 | 0.9 | 0.690 |
| C_0111 | Butyrylcarnitine | 1.3E−03 | 4.8E−04 | 1.6E−03 | 4.3E−04 | 0.9 | 0.460 |
| C_0101 | Carboxymethyllysine | 1.8E−04 | 2.2E−05 | 1.8E−04 | 3.1E−05 | 1.0 | 0.972 |
| C_0073 | Carnitine | 1.6E−02 | 2.8E−03 | 1.8E−02 | 3.0E−03 | 0.9 | 0.354 |
| C_0107 | Carnosine | 1.2E−04 | 1.1E−05 | 1.1E−04 | 1.1E−05 | 1.1 | 0.206 |
| A_0095 | Cholic acid | 5.0E−03 | 8.0E−03 | 3.8E−03 | 5.0E−03 | 1.3 | 0.833 |
| C_0014 | Choline | 1.1E−02 | 1.4E−03 | 1.0E−02 | 2.5E−03 | 1.1 | 0.679 |
| A_0044 | cis -Aconitic acid | 4.8E−03 | 3.8E−04 | 4.9E−03 | 5.1E−04 | 1.0 | 0.607 |
| A_0055 | Citric acid | 5.7E−02 | 5.4E−03 | 5.6E−02 | 6.8E−03 | 1.0 | 0.907 |
| C_0084 | Citrulline | 1.6E−02 | 1.5E−03 | 1.8E−02 | 3.0E−03 | 0.9 | 0.247 |
| C_0040 | Creatine | 4.4E−02 | 7.4E−03 | 5.6E−02 | 8.4E−03 | 0.8 | 0.054 |
| C_0021 | Creatinine | 2.4E−03 | 2.2E−04 | 3.0E−03 | 2.6E−04 | 0.8 | 0.004 ** |
| C_0106 | Cystathionine | 2.2E−04 | 3.0E−05 | 2.3E−04 | 4.8E−05 | 1.0 | 0.683 |
| C_0133 | Cysteine glutathione disulfide | 6.2E−03 | 2.6E−03 | 4.9E−03 | 8.0E−04 | 1.2 | 0.426 |
| C_0113 | Cystine | 6.1E−03 | 4.2E−04 | 4.9E−03 | 8.8E−04 | 1.2 | 0.024 * |
| C_0116 | Cytidine | 7.5E−04 | 7.9E−05 | 6.6E−04 | 1.6E−04 | 1.1 | 0.290 |
| C_0016 | Diethanolamine | 1.4E−04 | 4.1E−05 | 1.3E−04 | 6.9E−05 | 1.1 | 0.735 |
| C_0119 | Dyphylline | 2.8E−03 | 1.0E−03 | 2.7E−03 | 5.0E−04 | 1.0 | 0.859 |
| C_0058 | Ectoine | 3.4E−04 | 6.6E−05 | 5.0E−04 | 9.9E−05 | 0.7 | 0.026 * |
| C_0002 | Ethanolamine | 1.3E−03 | 7.6E−04 | 1.1E−03 | 3.2E−04 | 1.2 | 0.628 |
| A_0030 | Ethanolamine phosphate | 2.4E−04 | 2.4E−05 | 2.4E−04 | 1.2E−04 | 1.0 | 0.986 |

Putative Metabolites (2)

| | | | | | | | |
|---|---|---|---|---|---|---|---|
| A_0012 | Fumaric acid | 5.1E−04 | 2.1E−04 | 6.5E−04 | 1.1E−04 | 0.8 | 0.279 |
| C_0013 | GABA | 1.1E−04 | 1.3E−05 | 2.9E−04 | 1.7E−04 | 0.4 | 0.209 |
| C_0086 | Galactosamine | 8.3E−05 | 1.2E−06 | 1.1E−04 | 4.5E−05 | 0.8 | 0.250 |
| | Glucosamine | | | | | | |
| A_0098 | GDP | N.A. | N.A. | 3.5E−04 | N.A. | <1 | N.A. |
| C_0063 | Gln | 1.7E−01 | 2.3E−02 | 1.8E−01 | 3.7E−02 | 0.9 | 0.483 |
| C_0066 | Glu | 6.1E−03 | 2.8E−03 | 7.9E−03 | 3.7E−03 | 0.8 | 0.403 |
| A_0064 | Glucaric acid | 1.2E−04 | 4.1E−05 | 1.6E−04 | 4.7E−05 | 0.8 | 0.277 |
| A_0058 | Gluconic acid | 1.7E−03 | 1.2E−03 | 1.4E−03 | 3.5E−04 | 1.2 | 0.659 |
| C_0085 | Gluconolactone | 8.5E−04 | 5.6E−04 | 6.3E−04 | 1.6E−04 | 1.4 | 0.490 |
| A_0075 | Glucose 6-phosphate | 1.5E−04 | 4.2E−05 | 1.8E−04 | 1.0E−04 | 0.9 | 0.721 |
| A_0057 | Glucuronic acid | 3.6E−04 | 4.2E−05 | 3.2E−04 | 2.4E−05 | 1.1 | 0.165 |
| | Galacturonic acid | | | | | | |
| A_0023 | Glutaric acid | 2.7E−04 | 3.3E−05 | 2.7E−04 | 5.8E−05 | 1.0 | 0.869 |
| C_0129 | Glutathione | 6.2E−03 | 3.9E−03 | 5.3E−03 | 1.3E−03 | 1.2 | 0.682 |
| | (GSSG)_divalent | | | | | | |
| C_0004 | Gly | 4.6E−02 | 6.9E−03 | 4.5E−02 | 1.1E−02 | 1.0 | 0.846 |
| A_0010 | Glyceric acid | 5.7E−04 | 6.1E−05 | 5.4E−04 | 3.5E−05 | 1.0 | 0.540 |
| C_0010 | Glycerol | 7.3E−03 | 3.5E−03 | 6.7E−03 | 1.4E−03 | 1.1 | 0.738 |
| A_0040 | Glycerol 3-phosphate | 6.3E−04 | 1.3E−04 | 6.3E−04 | 9.2E−05 | 1.0 | 0.961 |
| C_0120 | Glycerophosphocholine | 9.8E−03 | 3.7E−03 | 1.2E−02 | 1.6E−03 | 0.8 | 0.383 |
| A_0002 | Glycolic acid | 3.2E−03 | 2.3E−04 | 3.2E−03 | 2.2E−04 | 1.0 | 0.777 |
| A_0001 | Glyoxylic acid | 4.1E−04 | 9.5E−05 | 4.5E−04 | 1.7E−04 | 0.9 | 0.633 |
| C_0082 | Gramine | 9.2E−05 | 7.3E−06 | 1.2E−04 | 2.1E−05 | 0.8 | 0.039 * |
| A_0106 | GTP | N.A. | N.A. | 3.4E−04 | N.A. | <1 | N.A. |
| C_0083 | Guanidinosuccinic acid | 9.8E−05 | 3.0E−05 | 9.9E−05 | 3.0E−05 | 1.0 | 0.963 |
| C_0023 | Guanidoacetic acid | 8.3E−04 | 4.1E−04 | 7.4E−04 | 2.8E−04 | 1.1 | 0.742 |
| A_0014 | Hexanoic acid | 1.3E−04 | 1.3E−05 | 1.2E−04 | 2.5E−05 | 1.1 | 0.502 |
| A_0047 | Hippuric acid | 3.6E−04 | 2.2E−04 | 7.9E−04 | 1.7E−04 | 0.5 | 0.154 |
| C_0070 | His | 1.6E−02 | 3.9E−03 | 2.0E−02 | 6.4E−03 | 0.8 | 0.317 |
| C_0019 | Histamine | 1.7E−04 | 8.9E−05 | 9.5E−05 | 5.0E−05 | 1.7 | 0.298 |
| C_0114 | Homocarnosine | 7.8E−05 | 9.8E−06 | 7.8E−05 | 1.8E−05 | 1.0 | 0.995 |
| C_0095 | Homocitrulline | 2.9E−04 | 5.2E−05 | 3.2E−04 | 3.2E−05 | 0.9 | 0.294 |
| C_0028 | Homoserine | 2.2E−04 | N.A. | 2.2E−04 | 5.3E−05 | 1.0 | N.A. |
| A_0049 | Homovanillic acid | 3.6E−04 | 2.0E−05 | 3.6E−04 | 4.4E−05 | 1.0 | 0.822 |
| C_0039 | Hydroxyproline | 4.5E−03 | 2.0E−03 | 4.7E−03 | 1.1E−03 | 0.9 | 0.841 |
| C_0017 | Hypotaurine | 8.5E−04 | 5.0E−04 | 1.2E−03 | 9.3E−04 | 0.7 | 0.524 |
| C_0042 | Ile | 5.7E−02 | 6.8E−03 | 6.5E−02 | 7.8E−03 | 0.9 | 0.098 |
| A_0019 | Isethionic acid | 1.2E−03 | 1.2E−04 | 1.1E−03 | 2.6E−04 | 1.2 | 0.208 |
| A_0004 | Isobutyric acid | | | | | | |
| | Butyric acid | 2.7E−04 | 1.7E−04 | 3.4E−04 | 2.5E−04 | 0.8 | 0.718 |
| C_0110 | Isobutyrylcarnitine | 2.8E−04 | 1.2E−04 | 3.3E−04 | 1.2E−04 | 0.8 | 0.488 |
| A_0054 | Isocitric acid | 4.3E−03 | 7.4E−04 | 4.3E−03 | 1.0E−03 | 1.0 | 0.990 |
| A_0041 | Isovalerylalanine-1 | 4.4E−04 | 1.2E−04 | 3.7E−04 | 1.1E−04 | 1.2 | 0.488 |
| | N-Acetylleucine-1 | | | | | | |
| A_0042 | Isovalerylalanine-2 | 1.7E−04 | 4.6E−05 | 2.3E−04 | 3.9E−05 | 0.7 | 0.133 |
| | N-Acetylleucine-2 | | | | | | |
| C_0118 | Isovalerylcarnitine | 1.5E−04 | N.A. | 2.5E−04 | 9.2E−05 | 0.6 | N.A. |
| C_0102 | Kynurenine | 2.4E−04 | 1.9E−05 | 2.9E−04 | 6.0E−05 | 0.9 | 0.165 |
| A_0005 | Lactic acid | 3.1E−01 | 6.6E−02 | 4.2E−01 | 7.2E−02 | 0.7 | 0.042 * |
| A_0060 | Lauric acid | 4.0E−04 | 2.6E−05 | 3.3E−04 | 3.3E−05 | 1.2 | 0.008 ** |
| C_0041 | Leu | 9.3E−02 | 1.3E−02 | 1.1E−01 | 1.6E−02 | 0.8 | 0.098 |
| C_0064 | Lys | 4.9E−02 | 4.9E−03 | 6.1E−02 | 1.1E−02 | 0.8 | 0.049 * |
| A_0026 | Malic acid | 7.9E−03 | 3.2E−03 | 1.0E−02 | 1.8E−03 | 0.8 | 0.306 |
| C_0067 | Met | 1.2E−02 | 2.5E−03 | 1.9E−02 | 4.3E−03 | 0.6 | 0.011 * |
| C_0076 | Methionine sulfoxide | 9.6E−04 | 4.4E−04 | 1.7E−03 | 5.7E−04 | 0.5 | 0.041 * |
| A_0065 | Mucic acid | 3.4E−04 | 5.1E−05 | 3.2E−04 | 8.1E−05 | 1.1 | 0.625 |
| C_0012 | N,N-Dimethylglycine | 1.7E−03 | 5.0E−04 | 1.7E−03 | 1.6E−04 | 1.0 | 0.895 |
| A_0022 | N-Acetylalanine | 1.6E−04 | 2.9E−05 | 1.4E−04 | 2.7E−05 | 1.2 | 0.238 |
| A_0045 | N-Acetylaspartic acid | 1.3E−04 | 1.8E−05 | 1.3E−04 | 1.0E−05 | 1.0 | 0.882 |
| C_0105 | N-Acetylgalactosamine | | | | | | |
| | N-Acetylmannosamine | 2.9E−04 | N.A. | 2.4E−04 | 2.5E−05 | 1.2 | N.A. |
| | N-Acetylglucosamine | | | | | | |
| A_0052 | N-Acetylglutamine | 1.9E−04 | 9.2E−05 | 1.1E−04 | 8.3E−06 | 1.7 | 0.444 |
| A_0015 | N-Acetylglycine | 6.9E−04 | 1.4E−04 | 4.4E−04 | 2.1E−04 | 1.6 | 0.057 |
| C_0096 | N-Acetylhistidine | 1.2E−04 | 2.8E−05 | 1.4E−04 | 2.8E−05 | 0.9 | 0.414 |
| C_0091 | N-Acetyllysine | N.A. | N.A. | 1.5E−04 | 2.7E−05 | <1 | N.A. |
| A_0063 | N-Acetylphenylalanine | 1.5E−04 | 3.7E−05 | 2.1E−04 | 2.5E−05 | 0.7 | 0.030 * |

Putative Metabolites (3)

| | | | | | | | |
|---|---|---|---|---|---|---|---|
| C_0065 | N-Acetylserine | 1.5E−04 | 3.2E−05 | 1.9E−04 | 3.7E−05 | 0.8 | 0.245 |
| A_0072 | N-Acetyltryptophan | 2.3E−04 | 6.6E−05 | 1.8E−04 | 3.4E−05 | 1.3 | 0.469 |
| C_0059 | N-Ethylmaleimide_ + H₂O | 3.0E−04 | N.A. | 1.4E−04 | N.A. | 2.1 | N.A. |
| A_0007 | N-Formylglycine | 8.3E−05 | 3.8E−05 | 1.0E−04 | 2.8E−05 | 0.8 | 0.590 |
| C_0038 | N-Methylproline | 2.5E−04 | 9.0E−05 | 2.3E−04 | 4.5E−05 | 1.1 | 0.630 |
| C_0069 | N¹-Methyl-4-pyridone-5-carboxamide | 6.3E−04 | 8.0E−05 | 4.8E−04 | 1.5E−04 | 1.3 | 0.082 |
| C_0080 | N⁵-Ethylglutamine | 1.7E−03 | 5.5E−04 | 2.0E−03 | 1.9E−04 | 0.9 | 0.439 |
| C_0094 | N⁶N⁶,N⁶-Trimethyllysine | 3.7E−04 | 6.9E−05 | 4.0E−04 | 1.3E−04 | 0.9 | 0.584 |
| C_0092 | N⁶-Acetyllysine | 2.5E−04 | 7.5E−06 | 3.0E−04 | 2.2E−05 | 0.8 | 0.002 ** |
| C_0071 | N⁶-Methyllysine | 1.5E−03 | 1.8E−04 | 1.9E−03 | 3.2E−04 | 0.8 | 0.090 |
| C_0090 | N⁸-Acetylspermidine | 5.6E−05 | 1.0E−05 | 4.8E−05 | 8.2E−06 | 1.2 | 0.222 |
| C_0032 | Nicotinamide | 8.6E−04 | 4.3E−04 | 8.7E−04 | 4.4E−04 | 1.0 | 0.999 |
| C_0093 | N_{ω}-Methylarginine | N.A. | N.A. | 7.9E−05 | 1.8E−05 | <1 | N.A. |
| C_0099 | O-Acetylcarnitine | 2.4E−02 | 3.1E−03 | 2.2E−02 | 3.9E−03 | 1.1 | 0.432 |
| C_0072 | O-Acetylhomoserine | 1.9E−03 | 8.4E−04 | 1.8E−03 | 4.4E−04 | 1.1 | 0.750 |
| | 2-Aminoadipic acid | | | | | | |
| A_0029 | o-Hydroxybenzoic acid | 2.0E−04 | N.A. | 3.9E−04 | 1.4E−04 | 0.5 | N.A. |
| C_0126 | Ophthalmic acid | 1.7E−04 | 1.0E−04 | 1.9E−04 | 9.0E−05 | 0.9 | 0.774 |
| C_0045 | Ornithine | 9.1E−03 | 9.5E−04 | 1.5E−02 | 5.2E−03 | 0.6 | 0.040 * |
| A_0048 | p-Hydroxyphenylpyruvic acid | 3.5E−04 | 1.4E−04 | 6.7E−04 | 2.3E−04 | 0.5 | 0.040 * |
| A_0068 | Pantothenic acid | 5.4E−04 | 2.3E−04 | 5.4E−04 | 2.0E−04 | 1.0 | 0.999 |
| C_0077 | Phe | 4.0E−02 | 5.6E−03 | 5.3E−02 | 1.5E−02 | 0.8 | 0.093 |
| A_0056 | Phenaceturic acid | 2.3E−04 | 1.0E−04 | 3.9E−04 | 1.4E−04 | 0.6 | 0.098 |
| A_0066 | Phosphocreatine | 9.7E−05 | 6.0E−06 | 1.0E−04 | 2.3E−05 | 0.9 | 0.596 |
| C_0089 | Phosphorylcholine | 4.4E−04 | 7.7E−05 | 5.5E−04 | 9.6E−05 | 0.8 | 0.086 |
| C_0033 | Picolinic acid | 8.0E−05 | 1.2E−05 | 1.2E−04 | 3.0E−05 | 0.7 | 0.319 |
| C_0037 | Pipecolic acid | 2.7E−03 | 8.5E−04 | 2.6E−03 | 4.7E−04 | 1.1 | 0.767 |
| C_0022 | Pro | 3.0E−02 | 8.3E−03 | 4.0E−02 | 1.5E−02 | 0.8 | 0.226 |
| C_0006 | Putrescine | N.A. | N.A. | 2.5E−04 | N.A. | <1 | N.A. |
| C_0078 | Pyridoxal | 1.2E−04 | 3.6E−05 | 1.1E−04 | 2.3E−05 | 1.1 | 0.724 |
| A_0046 | Pyrophosphate | 9.1E−04 | 9.2E−05 | 8.7E−04 | 7.4E−05 | 1.0 | 0.548 |
| A_0003 | | 5.8E−03 | 5.2E−04 | 7.5E−03 | 9.9E−04 | 0.8 | 0.009 ** |
| A_0071 | Ribulose 5-phosphate | 1.9E−04 | 3.0E−05 | 2.1E−04 | 5.8E−05 | 0.9 | 0.630 |
| C_0048 | S-Methylcysteine | 3.2E−04 | 1.7E−04 | 2.4E−04 | 8.1E−05 | 1.3 | 0.421 |
| C_0075 | S-Methylmethionine | 5.7E−05 | 6.5E−06 | 6.1E−05 | 1.7E−05 | 0.9 | 0.629 |
| A_0061 | S-Sulfocysteine | 4.4E−04 | 3.5E−04 | 4.3E−04 | 1.6E−04 | 1.0 | 0.959 |
| C_0008 | Sarcosine | 2.2E−03 | 5.0E−04 | 2.6E−03 | 6.9E−04 | 0.9 | 0.329 |
| C_0097 | SDMA | 7.6E−05 | 9.4E−06 | 6.5E−05 | 1.3E−05 | 1.2 | 0.179 |
| C_0015 | Ser | 1.8E−02 | 5.0E−03 | 2.3E−02 | 7.4E−03 | 0.8 | 0.258 |
| C_0062 | Spermidine | 3.0E−04 | 7.9E−05 | 3.0E−04 | 1.7E−04 | 1.0 | 0.999 |
| C_0060 | Stachydrine | 4.4E−03 | 9.1E−04 | 6.1E−03 | 1.8E−03 | 0.7 | 0.085 |
| A_0016 | Succinic acid | 1.3E−02 | 2.4E−03 | 1.5E−02 | 5.0E−03 | 0.8 | 0.365 |
| C_0034 | Taurine | 9.9E−03 | 8.4E−04 | 9.4E−03 | 1.5E−03 | 1.1 | 0.518 |
| A_0105 | Taurocholic acid | 1.0E−02 | 1.9E−02 | 3.2E−02 | 3.2E−02 | 0.3 | 0.207 |
| A_0033 | Terephthalic acid | 1.6E−04 | 2.3E−05 | 1.7E−04 | 2.5E−05 | 1.0 | 0.621 |
| C_0087 | Theobromine | 4.8E−04 | N.A. | 3.8E−04 | 4.6E−05 | 1.3 | N.A. |
| C_0121 | Thiamine | 1.4E−04 | 1.2E−05 | 1.8E−04 | 2.8E−05 | 0.8 | 0.016 * |
| C_0131 | Thiamine phosphate | 5.4E−05 | 1.1E−05 | 5.5E−05 | 7.7E−06 | 1.0 | 0.905 |
| C_0046 | Thiaproline | 1.3E−04 | 3.5E−05 | 1.3E−04 | 2.3E−05 | 1.0 | 0.835 |
| C_0027 | Thr | 3.0E−02 | 5.1E−03 | 3.6E−02 | 5.3E−03 | 0.8 | 0.107 |
| A_0028 | Threonic acid | 2.1E−03 | 4.3E−04 | 2.2E−03 | 3.6E−04 | 1.0 | 0.718 |
| C_0115 | Thymidine | 6.3E−04 | 8.4E−05 | 6.5E−04 | 1.6E−04 | 1.0 | 0.850 |
| C_0053 | Trigonelline | 1.1E−03 | 4.0E−04 | 1.8E−03 | 4.8E−04 | 0.6 | 0.032 * |
| C_0005 | Trimethylamine N-oxide | 1.3E−03 | 6.4E−04 | 1.3E−03 | 7.9E−04 | 1.0 | 0.970 |
| C_0100 | Trp | 2.8E−02 | 8.2E−03 | 3.6E−02 | 3.8E−03 | 0.8 | 0.164 |
| C_0088 | Tyr | 2.7E−02 | 8.0E−03 | 3.4E−02 | 7.7E−03 | 0.8 | 0.227 |
| C_0020 | Uracil | 3.0E−04 | 5.9E−05 | 3.0E−04 | 9.1E−05 | 1.0 | 0.914 |
| C_0001 | Urea | 7.8E−01 | 1.7E−01 | 8.1E−01 | 5.6E−02 | 1.0 | 0.753 |
| A_0027 | Ureidoglycolic acid | 1.4E−04 | 4.5E−05 | 2.1E−04 | 7.4E−05 | 0.7 | 0.206 |
| A_0037 | Uric acid | 6.0E−03 | 3.3E−04 | 5.6E−03 | 6.2E−04 | 1.1 | 0.228 |
| C_0117 | Uridine | 1.8E−03 | 4.6E−04 | 1.7E−03 | 4.9E−04 | 1.1 | 0.631 |

Putative Metabolites (4)

| | | | | | | | |
|---|---|---|---|---|---|---|---|
| C_0055 | Urocanic acid | 7.8E−05 | 7.0E−06 | 9.6E−05 | 1.9E−05 | 0.8 | 0.065 |
| A_0102 | UTP | N.A. | N.A. | 2.5E−04 | N.A. | <1 | N.A. |
| C_0024 | Val | 9.6E−02 | 1.4E−02 | 1.1E−01 | 1.5E−02 | 0.9 | 0.151 |
| A_0011 | XA0002 | 4.2E−04 | 1.4E−04 | 4.0E−04 | 1.1E−04 | 1.0 | 0.871 |
| A_0035 | XA0012 | 3.2E−04 | 5.7E−05 | 3.1E−04 | 9.8E−05 | 1.0 | 0.826 |
| A_0043 | XA0013 | 5.7E−04 | 2.0E−04 | 6.4E−04 | 3.2E−04 | 0.9 | 0.655 |
| A_0053 | XA0019 | 3.2E−04 | 8.9E−05 | 5.5E−04 | 9.6E−05 | 0.6 | 0.088 |
| A_0069 | XA0027 | 5.0E−04 | 8.0E−05 | 3.7E−04 | 8.9E−05 | 1.4 | 0.038 |
| A_0074 | XA0035 | 1.1E−03 | 3.2E−04 | 8.6E−04 | 4.4E−04 | 1.3 | 0.359 |
| A_0073 | XA0036 | 1.5E−04 | 3.0E−05 | 1.3E−04 | 1.9E−04 | 1.1 | 0.557 |
| C_0036 | XC0016 | 4.0E−04 | 7.1E−05 | 3.9E−04 | 4.1E−05 | 1.0 | 0.815 |
| C_0103 | XC0061 | 8.6E−04 | 3.4E−04 | 1.3E−03 | 5.8E−04 | 0.7 | 0.172 |
| C_0128 | XC0120 | 7.9E−05 | 9.4E−06 | 6.5E−05 | 1.6E−05 | 1.2 | 0.109 |
| C_0009 | β-Ala | 2.7E−04 | 1.1E−04 | 3.0E−04 | 1.8E−04 | 0.9 | 0.750 |
| A_0017 | β-Hydroxyisovaleric acid | 2.0E−04 | 3.9E−05 | 2.3E−04 | 6.0E−04 | 0.9 | 0.522 |
| C_0061 | γ-Butyrobetaine | 1.6E−03 | 1.1E−04 | 1.7E−03 | 4.8E−03 | 1.0 | 0.891 |

ID consists of analysis mode and number. ‘C’ and ‘A’ showed cation and anion modes, respectively.
N.D. (Not Detected): The target peak or metabolite was below detection limits.
N.A. (Not Available): The calculation was impossible because of insufficience of the data.
^{†}Putative metabolites which were assigned on the basis of m/z and MT in HMT standard compound library.
^{¶} The ratio is of computed by using averaged detection values. The latter was used as denominator.
^{∥} The p-value is computed by Welch's t-test. (* <0.05, ** <0.01, *** <0.001)
The data are sorted by Compound name in ascending order.

TABLE 8

| | | Concentration (μM) | | | | Comparative Analysis | |
|---|---|---|---|---|---|---|---|
| | | Control | | Treatment | | Control vs Treatment | |
| ID | Metabolite | Mean | S.D. | Mean | S.D. | Ratio ^{¶} | p-value ^{∥} |

Quantitative Estimation of Target Metabolites (1)

| | | | | | | | |
|---|---|---|---|---|---|---|---|
| A_0008 | 2-Hydroxybutyric acid | 27 | 10 | 25 | 4.9 | 1.1 | 0.720 |
| A_0032 | 2-Oxoglutanic acid | 47 | 29 | 72 | 35 | 0.7 | 0.276 |
| A_0013 | 2-Oxoisovaleric acid | 7.6 | 1.4 | 6.6 | 0.8 | 1.2 | 0.224 |
| A_0051 | 2-Phosphogluceric acid | N.A. | N.A. | N.A. | N.A. | N.A. | N.A. |
| A_0009 | 3-Hydrocybutric acid | 406 | 229 | 237 | 123 | 1.7 | 0.248 |
| A_0050 | 3-Phosphoglyceric acid | N.A. | N.A. | N.A. | N.A. | N.A. | N.A. |
| A_0078 | 6-Phosphogluconic acid | N.A. | N.A. | N.A. | N.A. | N.A. | N.A. |
| A_0094 | Acetyl CoA_divalent | N.A. | N.A. | N.A. | N.A. | N.A. | N.A. |
| C_0049 | Adennie | N.A. | N.A. | N.A. | N.A. | N.A. | N.A. |
| C_0122 | Adenosine | 0.11 | N.A. | 0.2 | 0.2 | 0.5 | N.A. |
| A_0097 | ADP | 1.0 | 0.2 | 4.7 | 8.9 | 0.2 | 0.404 |
| C_0007 | Ala | 282 | 34 | 355 | 75 | 0.8 | 0.076 |
| A_0086 | AMP | 3.4 | 1.2 | 5.1 | 6.6 | 0.7 | 0.599 |
| C_0052 | Anthranilic acid | N.A. | N.A. | N.A. | N.A. | N.A. | N.A. |
| C_0081 | Arg | 90 | 9.3 | 111 | 20 | 0.8 | 0.065 |
| C_0044 | Asn | 33 | 17 | 51 | 29 | 0.7 | 0.256 |
| C_0047 | Asp | 6.3 | 1.6 | 8.3 | 2.1 | 0.8 | 0.137 |
| A_0104 | ATP | 2.5 | 0.4 | 13 | 19 | 0.2 | 0.353 |
| C_0026 | Bataine | 68 | 20 | 75 | 34 | 0.9 | 0.690 |
| C_0029 | Betaine aldehyde_ + H₂O | N.A. | N.A. | N.A. | N.A. | N.A. | N.A. |
| A_0083 | cAMP | N.A. | N.A. | N.A. | N.A. | N.A. | N.A. |
| C_0107 | Carnosine | 0.6 | 0.06 | 0.6 | 0.05 | 1.1 | 0.206 |
| A_0092 | CDP | N.A. | N.A. | N.A. | N.A. | N.A. | N.A. |
| A_0085 | cGMP | N.A. | N.A. | N.A. | N.A. | N.A. | N.A. |
| C_0014 | Choline | 23 | 2.9 | 22 | 5.4 | 1.1 | 0.679 |
| A_0044 | cis -Aconitic acid | 23 | 1.8 | 23 | 2.4 | 1.0 | 0.607 |
| A_0155 | Citric acid | 335 | 32 | 332 | 40 | 1.0 | 0.907 |
| C_0084 | Citrulline | 63 | 5.8 | 70 | 11 | 0.9 | 0.247 |
| A_0081 | CMP | N.A. | N.A. | N.A. | N.A. | N.A. | N.A. |
| A_0089 | CoA_divalent | N.A. | N.A. | N.A. | N.A. | N.A. | N.A. |
| C_0040 | Creatine | 124 | 2.1 | 156 | 23 | 0.8 | 0.054 |
| C_0121 | Creatinine | 7.9 | 0.7 | 9.9 | 0.9 | 0.8 | 0.004 ** |
| A_0101 | CTP | N.A. | N.A. | N.A. | N.A. | N.A. | N.A. |
| C_0031 | Cys | N.A. | N.A. | N.A. | N.A. | N.A. | N.A. |
| C_0116 | Cytidine | 2.4 | 0.3 | 2.2 | 0.5 | 1.1 | 0.290 |
| C_0018 | Cytosine | N.A. | N.A. | N.A. | N.A. | N.A. | N.A. |
| A_0103 | dATP | N.A. | N.A. | N.A. | N.A. | N.A. | N.A. |
| A_0099 | dCTP | N.A. | N.A. | N.A. | N.A. | N.A. | N.A. |
| A_0038 | Dihydroxyacetone phosphate | N.A. | N.A. | N.A. | N.A. | N.A. | N.A. |
| A_0091 | dTDP | N.A. | N.A. | N.A. | N.A. | N.A. | N.A. |
| A_0080 | dTMP | N.A. | N.A. | N.A. | N.A. | N.A. | N.A. |
| A_0100 | dTTP | N.A. | N.A. | N.A. | N.A. | N.A. | N.A. |
| A_0059 | Erythrose 4-phosphate | N.A. | N.A. | N.A. | N.A. | N.A. | N.A. |
| A_0084 | Fructose 1,6-diphosphate | N.A. | N.A. | N.A. | N.A. | N.A. | N.A. |
| A_0077 | Fructose 6-phosphate | N.A. | N.A. | N.A. | N.A. | N.A. | N.A. |
| A_0012 | Fumaric acid | 7.5 | 3.1 | 9.5 | 1.5 | 0.8 | 0.279 |
| C_0013 | GABA | 0.4 | 0.05 | 1.0 | 0.6 | 0.4 | 0.209 |
| A_0098 | GDP | N.A. | N.A. | 3.0 | N.A. | <1 | N.A. |
| A_0063 | Gln | 672 | 94 | 729 | 148 | 0.9 | 0.483 |
| A_0066 | Glu | 25 | 11 | 32 | 15 | 0.8 | 0.403 |
| A_0058 | Gluconic acid | 18 | 12 | 1.5 | 3.6 | 1.2 | 0.659 |
| C_0076 | Glucose 1-phosphate | N.A. | N.A. | N.A. | N.A. | N.A. | N.A. |
| A_0075 | Glucose 6-phosphate | 2.3 | 0.6 | 2.6 | 1.6 | 0.9 | 0.721 |
| A_0130 | Glutathione (GSH) | N.A. | N.A. | N.A. | N.A. | N.A. | N.A. |
| A_0129 | Glutathione (GSSG)_divalent | 15 | 9.6 | 13 | 3.2 | 1.2 | 0.682 |
| C_0004 | Gly | 327 | 48 | 319 | 76 | 1.0 | 0.846 |
| C_0039 | Glyceraldehyde 3-phosphate | N.A. | N.A. | N.A. | N.A. | N.A. | N.A. |
| A_0040 | Glycerol 3-phosphate | 11 | 2.3 | 11 | 1.6 | 1.0 | 0.961 |
| A_0002 | Glycolic acid | 72 | 5.1 | 74 | 5.0 | 1.0 | 0.777 |
| A_0001 | Glyoxylic acid | 15 | 3.4 | 16 | 6.1 | 0.9 | 0.633 |

Quantitative Estimation of Target Metabolites (2)

| | | | | | | | |
|---|---|---|---|---|---|---|---|
| A_0088 | GMP | N.A. | N.A. | N.A. | N.A. | N.A. | N.A. |
| A_0106 | GTP | N.A. | N.A. | 4.5 | N.A. | <1 | N.A. |
| C_0068 | Guanine | N.A. | N.A. | N.A. | N.A. | N.A. | N.A. |
| C_0125 | Guanosine | N.A. | N.A. | N.A. | N.A. | N.A. | N.A. |
| C_0070 | His | 58 | 14 | 70 | 23 | 0.8 | 0.317 |
| C_0028 | Homoserine | 0.9 | N.A. | 0.9 | 0.2 | 1.0 | N.A. |
| C_0039 | Hydroxyproline | 17 | 7.7 | 18 | 4.1 | 0.9 | 0.841 |
| C_0050 | Hypoxanthine | N.A. | N.A. | N.A. | N.A. | N.A. | N.A. |
| C_0042 | Ile | 84 | 10 | 97 | 12 | 0.9 | 0.098 |
| A_0087 | IMP | N.A. | N.A. | N.A. | N.A. | N.A. | N.A. |
| C_0123 | Inosine | N.A. | N.A. | N.A. | N.A. | N.A. | N.A. |
| A_0054 | Isocitric acid | 26 | 4.4 | 26 | 6.2 | 1.0 | 0.990 |
| A_0005 | Lactic acid | 4,704 | 1,000 | 6,362 | 1,088 | 0.7 | 0.042 * |
| C_0041 | Leu | 125 | 17 | 147 | 21 | 0.8 | 0.098 |
| C_0064 | Lys | 230 | 23 | 287 | 52 | 0.8 | 0.049 * |
| A_0026 | Malic acid | 60 | 24 | 75 | 14 | 0.8 | 0.306 |
| A_0096 | Malonyl CoA_divalent | N.A. | N.A. | N.A. | N.A. | N.A. | N.A. |
| C_0067 | Met | 32 | 6.9 | 51 | 12 | 0.6 | 0.011 * |
| C_0012 | N,N-Dimethylglycine | 5.8 | 1.7 | 5.7 | 0.6 | 1.0 | 0.895 |
| A_0108 | NAD+ | N.A. | N.A. | N.A. | N.A. | N.A. | N.A. |
| A_0109 | NADP+ | N.A. | N.A. | N.A. | N.A. | N.A. | N.A. |
| C_0045 | Ornithine | 40 | 4.2 | 66 | 23 | 0.6 | 0.040 * |
| C_0077 | Phe | 76 | 11 | 101 | 28 | 0.8 | 0.093 |
| A_0036 | Phosphoenolpyruvic acid | N.A. | N.A. | N.A. | N.A. | N.A. | N.A. |
| C_0022 | Pro | 73 | 20 | 96 | 35 | 0.8 | 0.226 |
| A_0090 | PRPP | N.A. | N.A. | N.A. | N.A. | N.A. | N.A. |
| C_0006 | Putrescine | N.A. | N.A. | 1.7 | N.A. | 51 | N.A. |
| A_0003 | Pyruvic acid | 137 | 12 | 177 | 23 | 0.8 | 0.009 ** |
| A_0070 | Ribose 5-phosphate | N.A. | N.A. | N.A. | N.A. | N.A. | N.A. |
| A_0071 | Ribulose 5-phosphate | 2.8 | 0.4 | 3.0 | 0.8 | 0.9 | 0.630 |
| C_0132 | S-Adenosylmethionine | N.A. | N.A. | N.A. | N.A. | N.A. | N.A. |
| C_0008 | Sarcosine | 8.4 | 1.9 | 9.8 | 2.6 | 0.9 | 0.329 |
| A_0079 | Sedoheptulose 7-phosphate | N.A. | N.A. | N.A. | N.A. | N.A. | N.A. |
| C_0015 | Ser | 103 | 28 | 129 | 41 | 0.8 | 0.258 |
| C_0062 | Spermidine | 1.2 | 0.3 | 1.2 | 0.7 | 1.0 | 0.999 |
| C_0098 | Spermine | N.A. | N.A. | N.A. | N.A. | N.A. | N.A. |
| A_0016 | Succinic acid | 139 | 27 | 164 | 55 | 0.8 | 0.365 |
| C_0027 | Thr | 122 | 21 | 148 | 22 | 0.8 | 0.107 |
| C_0115 | Thymidine | 3.9 | 0.5 | 4.0 | 1.0 | 1.0 | 0.850 |
| C_0035 | Thymine | N.A. | N.A. | N.A. | N.A. | N.A. | N.A. |
| C_0100 | Trp | 70 | 20 | 88 | 9.4 | 0.8 | 0.164 |
| C_0088 | Tyr | 76 | 23 | 95 | 22 | 0.8 | 0.227 |
| C_0054 | Tyramine | N.A. | N.A. | N.A. | N.A. | N.A. | N.A. |
| A_0093 | UDP | N.A. | N.A. | N.A. | N.A. | N.A. | N.A. |
| A_0082 | UMP | N.A. | N.A. | N.A. | N.A. | N.A. | N.A |
| C_0020 | Uracil | 4.7 | 0.9 | 4.6 | 1.4 | 1.0 | 0.914 |
| C_0117 | Uridine | 15 | 3.6 | 13 | 3.9 | 1.1 | 0.631 |
| A_0102 | UTP | N.A. | N.A. | 2.2 | N.A. | <1 | N.A. |
| C_0024 | Val | 191 | 29 | 222 | 30 | 0.9 | 0.151 |
| C_0009 | β-Ala | 1.2 | 0.5 | 1.3 | 0.8 | 0.9 | 0.750 |

ID consists of analysis mode and number. ‘C’ and ‘A’ showed cation and anion modes, respectively.
N.D. (Not Detected): The target peak or metabolite was below detection limits.
N.A. (Not Available): The calculation was impossible because of insufficience of the data.
^{¶} The ratio is of computed by using averaged detection values. The latter was used as denominator.
^{∥} The p-value is computed by Welch's t-test. (* <0.05, ** <0.01, *** <0.001)
The data are sorted by Compound name in ascending order.
ID consists of analysis mode and number. ‘C’ and ‘A’ showed cation and anion modes, respectively.
N.D. (Not Detected): The target peak or metabolite was below detection limits.
N.A. (Not Available): The calculation was impossible because of insufficience of the data.
† Putative metabolites which were assigned on the basis of m/z and MT in HMT standard compound library.
^{¶} The ratio is of computed by using averaged detection values. The latter was used as denominator.
^{∥} The p-value is computed by Welch's t-test. (* <0.05, ** <0.01, *** <0.001)
The data are sorted by Compound name in ascending order.

1. A method of treating or preventing an eye disorder, the method comprising:
administering to a subject in need thereof a composition comprising one or more microbial strains, components thereof, or metabolites thereof.

2. (canceled)

3. The method of claim 1, wherein the eye disorder is Age-related Macular Degeneration (AMD), Geographic atrophy, intermediate AMD, diabetic retinopathy, retinopathy of prematurity, retnitis pigmentosa, retinitis, glaucoma, proliferative vitreoretinopathy, uveitis, keratitis, or scleritis.

4. The method of claim 1, wherein the eye disorder is AMD.

5. The method of claim 1, wherein the subject is a mammal.

6. The method of claim 1, wherein the subject is a human.

7. The method of claim 1, wherein the one or more microbial strains are from a mammalian microbiome.

8. The method of claim 1, wherein the one or more microbial strains are from a human microbiome.

9. The method of claim 7, wherein the human microbiome is the microbiome of the subject.

10. The method of claim 1, wherein the one or more components or metabolites of the one or more microbial strains are selected from Appendix 1.

11. The method of claim 1, wherein the one or more components or metabolites of the one or more microbial strains is 2-keto-gluconate or 5-keto-gluconate.

12. (canceled)

13. The method of claim 1, wherein the one or more microbial strains are Gluconacetobacter hansenii, Terrisporobacter glycolicus, Coprococcus sp., Lactobacillus plantarum, Clostridium butyricum, Paenibacillus sp., Veillonella sp., Bifidobacterium sp., Bacillus subtilis, Acidaminococcus sp., or a combination thereof.

14. The method of claim 1, wherein the one or more microbial strains are Gluconacetobacter hanseni, Terrisporobacter glycolicus, Coprococcus sp., Lactobacillus plantarum, Veillonella atypica, Bifidobacterium, or a combination thereof.

15. (canceled)

16. The method of claim 1, wherein the composition comprises two or more microbial strains.

17.-18. (canceled)

19. The method of claim 1, wherein the composition is administered topically, orally, opthalmically, intravitreally, or suprachoroidally.

20.-21. (canceled)

22. The method of claim 1, wherein the composition is formulated as a syrup, a liquid, a tablet, a troche, a gummy, a capsule, a powder, a gel, a film, an injection, or an eye drop.

23. The method of claim 1, wherein each microbial strain of the one or more microbial strains is available at a concentration from 10¹ to 10¹⁵ CFU.

24. The method of any claim 1, wherein each microbial strain of the one or more microbial strains is available at a concentration of at least 10⁶ CFU.

25. A composition comprising one or more microbial strains, components thereof, or metabolites thereof, wherein the composition is for treating an eye disorder.

26. (canceled)

27. The composition of claim 25, wherein the one or more microbial strains are from a mammalian microbiome.

28. The composition of claim 25, wherein the one or more microbial strains are from a human microbiome.

29. The composition of claim 28, wherein the human microbiome is the microbiome of the subject.

30. The composition of claim 25, wherein the one or more components or metabolites are selected from Appendix 1.

31. The composition of claim 25, wherein the one or more components or metabolites is 2-keto-gluconate or 5-keto-gluconate.

32. (canceled)

33. The composition of claim 25, wherein the one or more microbial strains are Gluconacetobacter hansenii, Terrisporobacter glycolicus, Coprococcus sp., Lactobacillus plantarum, Clostridium butyricum, Paenibacillus sp., Veillonella sp., Bifidobacterium sp., Bacillus subtilis, Acidaminococcus sp., or a combination thereof.

34. The composition of claim 25, wherein the one or more microbial strains are Gluconacetobacter hanseni, Terrisporobacter glycolicus, Coprococcus sp., Lactobacillus plantarum, Veillonella atypica, Bifidobacterium, or a combination thereof.

35. (canceled)

36. The composition of claim 25, wherein the composition comprises two or more microbial strains.

37.-38. (canceled)

39. The composition of claim 25, wherein the composition is for topical, oral, opthalmical, intravitreal, or suprachoroidal administration.

40.-41. (canceled)

42. The composition of claim 25, wherein the composition is formulated as a syrup, a liquid, a tablet, a troche, a gummy, a capsule, a powder, a gel, a film, an injection, or an eye drop.

43. The composition of claim 25, wherein each microbial strain of the one or more microbial strains is available at a concentration from 10¹ to 10¹⁵ CFU.

44. The composition of claim 25, wherein each microbial strain of the one or more microbial strains is available at a concentration of at least 10⁶ CFU.

45.-95. (canceled)
